(19) United States
(12) Patent Application Publication
(10) Pub. No.: US 2010/0056371 A1
(43) Pub. Date: Mar. 4, 2010

(54) Use of Azolopyrimidines for Controlling Phytopathogenic Harmful Fungi

(75) Inventors: Jochen Dietz, Mannheim (DE); Wassilios Grammenos, Ludwigshafen (DE); Bernd Müller, Frankenthal (DE); Jan Klaas Lohmann, Ludwigshafen (DE); Jens Renner, Bad Durkheim (DE); Sarah Ulmschneider, Bad Durkheim (DE); Marianna Vrettou, Mannheim (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/522,345

(22) PCT Filed: Jan. 7, 2008
(86) PCT No.: PCT/EP08/50093

§ 371 Date: Jul. 7, 2009

(30) Foreign Application Priority Data
Jan. 8, 2007 (EP) 07100233.1

(51) Int. Cl. A01N 43/90; A01N 25/26; C07D 487/04; A01P 3/00

(52) U.S. Cl. 504/100; 514/259.31; 544/263

(57) ABSTRACT

Azolopyrimidines of the formula I

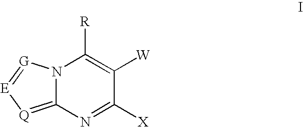

in which the symbols are each defined as specified in the description are suitable for controlling phytopathogenic harmful fungi.

The invention relates to the use of particular azolopyrimidines for controlling harmful fungi, to a method for controlling phytopathogenic harmful fungi, to novel fungicidal azolopyrimidine compounds, to processes for their preparation and to compositions comprising these compounds.

EP-A550 113 and WO 99/48893 disclose 6-phenyl-7-aminotriazolopyrimidines in general terms. WO 03/004465 discloses triazolopyrimidines which are substituted by groups bonded via carbon in the 5 and 7 positions. WO 02/002563 describes particular 6-phenyltriazolopyrimidines as fungicidally and pharmaceutically active. WO 05/000851 discloses 5-halo-7-aminopyrazolopyrimidines in general terms, which are substituted by a heterocycle in the 6 position.

These compounds are known for the control of harmful fungi.

However, the known compounds are not completely satisfactory in many cases, in terms, for example, of application rate, spectrum of activity, duration of activity, tendency to form resistance or economic aspects of the preparation process.

It is therefore a constant object to provide further fungicidal azolopyrimidines which, at least in some aspects, offer advantages over the known compounds.

It has been found that particular azolopyrimidines which have cycloalkyl or halogenated amino substituents in the 7 position are particularly suitable for controlling phytopathogenic harmful fungi.

Some compounds of this type are known from WO 2002/002563 and WO 2005/030775 as anticancer agents.

The invention therefore provides for the use of azolopyrimidines of the formula I

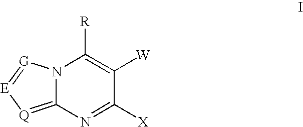

and their agriculturally acceptable salts for controlling phytopathogenic harmful fungi, where the substituents in the formula (I) are each defined as follows:
G, E, Q a) G is N; E is C—W² and Q is N or C—W³;
   b) G is C—W¹; E is C—W² and Q is N; or
   c) G is C—W¹; E is N and Q is C—W³;
W¹, W², W³ are each independently hydrogen, halogen, cyano, nitro, C₁-C₄-alkyl, C₂-C₆-alkenyl, C₂-C₆-alkynyl, C₁-C₄-haloalkyl, hydroxy-C₁-C₄-alkyl, C₁-C₄-alkoxy-C₁-C₄-alkyl, C₂-C₆-haloalkenyl, C₂-C₆-haloalkynyl, C₃-C₆-cycloalkyl, C₃-C₆-halocycloalkyl, C₁-C₄-alkoxy, C₁-C₄-haloalkoxy, C₁-C₄-alkylthio, C₁-C₄-alkylsulfinyl or C₁-C₄-alkylsulfonyl, formyl, thiocarbamoyl, C₁-C₄-alkylcarbonyl, C₁-C₄-alkoxycarbonyl, C₁-C₄-alkylaminocarbonyl, aminocarbonyl, di(C₁-C₄-alkyl)aminocarbonyl, C₁-C₄-alkoximinoalkyl, hydroximinoalkyl, CR¹⁰R¹¹R¹², C(R¹³)═NR¹⁴;
   R¹⁰, R¹¹, R¹² are each independently hydrogen, C₁-C₈-alkyl, C₃-C₆-cycloalkyl, C₁-C₈-alkoxy-C₁-C₈-alkyl, C₂-C₈-alkenyl, C₂-C₈-alkynyl, benzyl;
   R¹¹ and R¹² may together be oxy-C₁-C₅-alkyleneoxy in which the carbon chain may be substituted by from one to three groups from methyl, ethyl, hydroxyl, methoxy, ethoxy, hydroxymethyl, methoxymethyl, ethoxymethyl;
   R¹³ is hydrogen or C₁-C₈-alkyl;
   R¹⁴ is C₁-C₈-alkyl, C₃-C₆-cycloalkyl, phenyl, phenylamino, where the phenyl groups may be substituted by from one to five R^{b} groups;
R is NR¹R², C₃-C₆-cycloalkyl or C₃-C₁₂-halocycloalkyl; where R may comprise one, two, three or four identical or different R^{a} groups which are each independently selected from:
   R^{a} is R^{b}, carboxyl, OC(O)OR^{Π} or C₁-C₆-alkylthio;
   R^{b} is R^{c}, hydroxyl, C₁-C₆-alkoxy, C₂-C₆-alkenyloxy, C₉-C₆-alkynyloxy, C₃-C₆-cycloalkoxy or C₃-C₆-cycloalkenyloxy;
   R^{c} is hydrogen, cyano, C₁-C₆-alkyl, C₂-C₆-alkenyl, C₂-C₆-alkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, amino, C₁-C₆-alkylamino, di-C₁-C₆-alkylamino, aminocarbonyl, C(O)NHR^{Π}, C(O)NR^{Π}₂, phenyl, naphthyl, five-, six-, seven-, eight-, nine- or ten-membered saturated, partially unsaturated or aromatic heterocycle comprising one, two, three or four heteroatoms from the group of O, N and S,
      or two R^{a}, R^{b}, R^{C}, R^{Π}, R^{d} and/or R^{dd} groups form, together with the atom or the atoms to which they are bonded, a 3- to 12-membered saturated, partially unsaturated or aromatic ring which is carbocyclic or comprises from one to four heteroatoms from the group of N, O and S and which is unsubstituted or substituted by from 1 to 4 R^{d} radicals, in the case of halogen even up to the maximum number;
      R^{Π} is C₁-C₈-alkyl, C₉-C₈-alkenyl, C₃-C₈-alkynyl, C₃-C₆-cycloalkyl or C₃-C₆-cycloalkenyl;
      where the aliphatic, alicyclic or aromatic groups in the aforementioned R^{a}, R^{b},
      R^{c} and R^{Π} groups may in turn be partially or fully halogenated and/or bear one, two or three R^{d} groups:
      R^{d} is halogen, cyano, nitro, hydroxyl, mercapto, amino, carboxyl, alkyl, haloalkyl, alkenyl, alkynyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoxyimino (═N—O—C₁-C₈-alkyl), C₃-C₈-alkenyloximino (═N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms; cycloalkyl, cycloalkoxy, heterocyclyl, heterocyclyloxy, where the cyclic systems comprise from 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals comprise preferably from 6 to 10 ring members, the hetaryl radicals 5 or 6 ring members, where the cyclic systems are partially or completely halogenated and/or may bear from 1 to 3 R^{dd} groups;
      R^{dd} is halogen, cyano, nitro, hydroxyl, mercapto, amino, carboxyl, alkyl, haloalkyl, alkenyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoxyimino (═N—O—C₁-C₈-alkyl), C₃-C₈-alkenyloximino (═N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms;
R¹ is C₁-C₁₂-haloalkyl, C₂-C₁₂-haloalkenyl, C₂-C₁₂-haloalkynyl;
R² is H, R¹, C₁-C₁₂-alkyl, C₂-C₁₂-alkenyl, C₂-C₁₂-alkynyl, C₃-C₈-cycloalkyl, C₃-C₈-halocycloalkyl, C₃-C₆-cycloalkenyl, C₃-C₆-halocycloalkenyl, C₁-C₈-alkoxy, C₂-C₈-alkenyloxy, C₂-C₈-alkynyloxy, C₃-C₈-cycloalkoxy, NH₂, C₁-C₈-alkylamino, di-C₁-C₈-alkylamino, phenyl, naphthyl or a five- or six-membered saturated, partially unsaturated or aromatic heterocycle comprising one, two, three or four heteroatoms from the group of O, N and S, or Z—Y—(CR⁷R⁸)O(CR⁵R⁶)_{q}—CR³R⁴# where # is the bonding site to the nitrogen atom and:
   R³, R⁴, R⁵, R⁶, R⁷, R⁸ are each independently hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₈-halocycloalkyl, C₃-C₆-cycloalkenyl, C₃-C₆-halocycloalkenyl, phenyl, naphthyl or a five- or six-membered saturated, partially unsaturated or aromatic heterocycle comprising one, two, three or four heteroatoms from the group of O, N and S,
   R⁵ may also, with R³ or R⁷, together with the atoms to which these radicals are bonded, form a five-, six-, seven-, eight-, nine- or ten-membered saturated or partially unsaturated ring which, as well as carbon atoms, may comprise one, two or three heteroatoms from the group of O, N and S as ring members and/or may bear one or more substituents R^{a};
   R³ with R⁴, R⁵ with R⁶, R⁷ with R⁸ may each together also form carbonyl groups by being oxygen and form spiro groups by forming a C₂-C₅-alkylene or alkenylene, alkynylene chain which may be interrupted by one, two or three heteroatoms from the group of O, N and S;
   R¹ to R⁸ may each independently bear one, two, three or four identical or different R^{a} groups;
   Y is oxygen or sulfur;
   Z is hydrogen, carboxyl, formyl, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, C(NR^{A})SR^{Π}, C(S)R^{Π}, C(NR^{Π})NR^{A}R^{B}, C(NR^{Π})R^{A}, C(NR^{Π})OR^{A}, C(O)NR^{A}R^{B}, C(S)NR^{A}R^{B}, C₁-C₈-alkylsulfinyl, C₁-C₈-alkylthio, C₁-C₈-alkylsulfonyl, C(O)—C₁-C₄-alkylene-NR^{A}C(NR^{Π})NR^{A}R^{B},
      C(S)—C₁-C₄-alkylene-NR^{A}C(NR^{Π})NR^{A}R^{B},
      C(NR^{Π})—C₁-C₄-alkylene-NR^{A}C(NR^{Π})NR^{A}R^{B}, phenyl, naphthyl, five-, six-, seven-, eight-, nine- or ten-membered saturated, partially unsaturated or aromatic heterocycle which comprises one, two, three or four heteroatoms from the group of O, N and S and is bonded directly or via a carbonyl, thiocarbonyl, C₁-C₄-alkylcarbonyl or C₁-C₄-alkylthiocarbonyl group; where the carbon chains in the Z group may be substituted by one or more R^{b} groups;
      R^{A}, R^{B} are each independently hydrogen, C₂-alkenyl, C₂-alkynyl or one of the groups mentioned for R^{Π};
      R^{A} and R^{B}, together with the nitrogen atom to which they are bonded, or R^{A} and R^{Π} together with the carbon atoms and heteroatoms via which they are bonded, may also form a five- or six-membered saturated, partly unsaturated or aromatic ring which, as well as carbon atoms, may comprise one, two or three further heteroatoms from the group of O, N and S as ring members and/or may bear one or more substituents R^{a};
         or
         Z may also, with R⁶ or R⁸, form a five- or six-membered saturated or partly unsaturated ring which, as well as carbon atoms and Y, may comprise one or two further heteroatoms from the group of N and S as ring members and/or may bear one or more substituents R^{a} as defined below;
         the Z group may be partially or completely halogenated and/or bear one, two or three R^{b} groups;
W is phenyl or five- or six-membered heteroaryl which, as well as carbon atoms, comprises one, two or three further heteroatoms from the group of O, N and S as ring members, where the ring systems, as well as Lₘ groups, bear at least one substituent P¹,
   P¹ is Y¹—Y²-T;
      Y¹ is CR^{a}R^{a′}, C(O)O, C(O)NR^{b}, O, NR^{b} or S(O)ᵣ;
      Y² is C₃-C₈-alkylene, C₂-C₈-alkenylene, C₂-C₈-alkynylene, C₃-C₈-alkylene-(Y³—(C₂-C₄-alkylene))ₛ, C₃-C₈-alkenylene-(Y³—(C₂-C₄-alkylene))ₛ, C₃-C₈-alkynylene-(Y³—(C₂-C₄-alkylene))ₛ, and, if R is C₃-C₆-cycloalkyl or C₃-C₁₂-halocycloalkyl and/or Y¹ is CR^{a}R^{a′}, C(O)O, CONR^{b}, NR^{b} or S(O)ᵣ, is also C₁-C₂-alkylene, and/or, if T is OC(O)R^{a}, NR^{b}R^{b′}, C(O)NR^{b}R^{b′}, C(NOR^{c})R^{a} or T¹-C(=T²)-T³, is also C₁-C₂-alkylene;
      Y³ is O, S, NR^{b}
      r is 0, 1 or 2
      s is 1 or 2, preferably 1
      T is OR^{c}, OC(O)R^{a}, NR^{b}R^{b′}, C(O)NR^{b}R^{b′}, C(NOR^{c})R^{a}, T¹-C(=T²)-T³, and, if R is defined as NR¹R² or C₃-C₁₂-halocycloalkyl, is also C(O)OR^{c} or C(OR^{c})₂R^{a};
         T¹ is O, NR^{b};
         T² is O, S, NR^{b};
         T³ is R^{a}, OR^{c}, SR^{c}, NR^{b}R^{b′},
         where R^{a′} and R^{b′}, independently of R^{a} and R^{b} respectively, are each as defined for these radicals;
   L is halogen, hydroxyl, cyanato (OCN), cyano, nitro, C₁-C₈-alkyl, C₁-C₈-haloalkyl, C₂-C₁₀-alkenyl, C₂-C₁₀-haloalkenyl, C₂-C₁₀-alkynyl, C₃-C₆-cycloalkyl, C₃-C₆-halocycloalkyl, C₃-C₆-cycloalkenyl, C₁-C₈-alkoxy, C₁-C₈-haloalkoxy, C₂-C₁₀-alkenyloxy, C₂-C₁₀-alkynyloxy, C₃-C₆-cycloalkyloxy, C₃-C₆-cycloalkenyloxy, amino, C₁-C₄-alkylamino, di-(C₁-C₄)-alkylamino, C₁-C₄-alkylcarbonylamino, C(O)—R^{Φ}, C(S)—R^{Φ}, S(O)_{Π}—R^{Φ}; C₁-C₈-alkoxyimino-(C₁-C₈)-alkyl, C₂-C₁₁-alkenyloxyimino-(C₁-C₈)-alkyl, C₂-C₁₀-alkynyloxyimino-(C₁-C₈)-alkyl, C₂-C₁₀-alkynylcarbonyl, C₃-C₆-cycloalkylcarbonyl, or a five-, six-, seven-, eight-, nine- or ten-membered saturated, partly unsaturated or aromatic heterocycle comprising one, two, three or four heteroatoms from the group of O, N and S;
      R^{Φ} is hydrogen, C₁-C₄-alkyl, C₁-C₂-haloalkyl, C₁-C₄-alkoxy, C₂-C₄-alkenyloxy, C₂-C₄-alkynyloxy, amino, C₁-C₄-alkylamino, di-C₁-C₄-alkylamino; where the R^{Φ} groups may be substituted by one, two or three identical or different R^{b} groups as defined above;
      n is zero, 1 or 2;
   m is zero, 1, 2, 3, 4 or 5 and
X is halogen, cyano, C₁-C₄-alkyl, C₁-C₄-haloalkyl, C₁-C₄-alkoxy or C₁-C₄-haloalkoxy.

In addition, methods have been found for controlling harmful fungi, especially phytopathogenic harmful fungi, with compounds I.

The inventive compounds can be obtained by various routes. If R in formula I is NR¹R², the compounds can be prepared by reacting an aminoazole of the formula II with appropriately substituted phenylmalonates of the formula III in which R″ is alkyl, preferably C₁-C₆-alkyl, in particular methyl or ethyl.

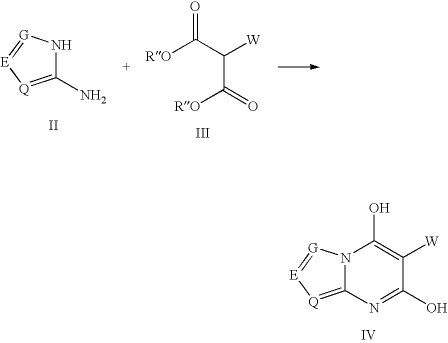

This reaction is usually carried out at temperatures of from 80° C. to 250° C., preferably from 120° C. to 180° C., without solvent or in an inert organic solvent in the presence of a base [cf. EP-A 770 615] or in the presence of acetic acid under the conditions known from Adv. Het. Chem. Vol. 57, p. 81 ff. (1993).

Suitable solvents are aliphatic hydrocarbons, aromatic hydrocarbons, such as toluene, o-, m- and p-xylene, halogenated hydrocarbons, ethers, nitriles, ketones, alcohols, and also N-methylpyrrolidone, dimethyl sulfoxide, dimethylformamide and dimethylacetamide. More preferably, the reaction is carried out without solvent or in chlorobenzene, xylene, dimethyl sulfoxide, N-methylpyrrolidone. It is also possible to use mixtures of the solvents mentioned.

Suitable bases are, in general, inorganic compounds, such as alkali metal and alkaline earth metal hydroxides, alkali metal and alkaline earth metal oxides, alkali metal and alkaline earth metal hydrides, alkali metal amides, alkali metal and alkaline earth metal carbonates and also alkali metal bicarbonates, organometallic compounds, in particular alkali metal alkyls, alkylmagnesium halides and also alkali metal and alkaline earth metal alkoxides and dimethoxymagnesium, moreover organic bases, for example tertiary amines, such as trimethylamine, triethylamine, diisopropylethylamine, tributylamine and N-methylpiperidine, N-methylmorpholine, pyridine, substituted pyridines, such as collidine, lutidine and 4-dimethylaminopyridine, and also bicyclic amines. Particular preference is given to using tertiary amines, such as diisopropylethylamine, tributylamine, N-methylmorpholine or N-methylpiperidine.

The bases are generally employed in catalytic amounts, but they can also be used in equimolar amounts, in excess or, if appropriate, as solvent.

The starting materials are generally reacted with one another in equimolar amounts. In terms of yield it may be advantageous to use an excess of base and the malonate III, based on the triazole.

Advantageously, the malonates of the formula III are obtained by reacting appropriately substituted bromoaromatic compounds with dialkyl malonates under Cu(I) catalysis [cf. Chemistry Letters, pp. 367-370, 1981; EP-A 10 02 788].

Alternatively, the malonates of the formula III can be constructed according to the scheme below under generally known conditions [cf.: March, Advanced Organic Chemistry, 3rd ed., p. 792 ff, J. Wiley & Sons, New York (1985)]:

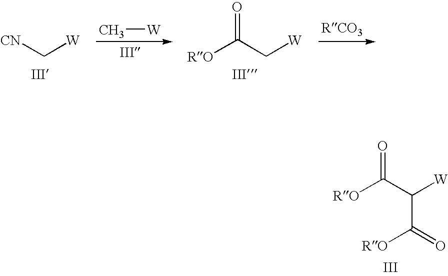

These reactions are usually carried out at temperatures of from −100° C. to +200° C., preferably from +20° C. to +100° C., in an inert organic solvent in the presence of a base [cf. U.S. Pat. No. 4,454,158; Bioorgan. & Med. Chem. Lett. Vol. 15, p. 2970 (2005); Organ. Proc. Res. & Develop., Vol. 8, p. 411 (2004); J. Am. Chem. Soc, Vol. 125, p. 13948 (2003); Ann. Pharm. Fr., Vol. 60, p. 314 (2004); Pharmazie, Vol. 44, p. 115 (1989)].

Under the conditions known from WO-A 94/20501, the dihydroxyazolopyrimidines of the formula IV are converted into the dihaloazolopyrimidines of the formula V in which Y is a halogen atom, preferably a bromine or a chlorine atom, in particular a chlorine atom. The halogenating agent [HAL] used is advantageously a chlorinating agent or a brominating agent, such as phosphorus oxybromide or phosphorus oxychloride, if appropriate in the presence of a solvent.

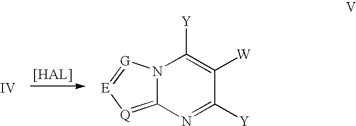

This reaction is usually carried out at from 0° C. to 150° C., preferably at from 80° C. to 125° C. [cf. EP-A 770 615].

Dihaloazolopyrimidines of the formula V are prepared using amines of the formula VI in which the variables are as defined for formula I.

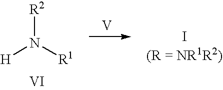

This reaction is advantageously carried out at from 0° C. to 70° C., preferably from 10° C. to 35° C., preferably in the presence of an inert solvent, such as an ether, for example dioxane, diethyl ether or, in particular, tetrahydrofuran, a halogenated hydrocarbon, such as dichloromethane, or an aromatic hydrocarbon, for example toluene [cf. WO 05/000851].

The use of a base, such as a tertiary amine, for example triethylamine, or an inorganic amine, such as potassium carbonate, is preferred; it is also possible for excess amine of the formula VI to serve as base.

Thus, using the 5,7-dichloroazolopyrimidines known from the publication mentioned at the outset, it is possible to obtain the 5-chloroazolopyrimidines of the formula I. They form a preferred part of the subject matter of the invention. Other 5,7-dihaloazolopyrimidines can be obtained analogously to the literature cited.

Amines of the formula VI are known from the literature, can be prepared by known methods or are commercially available.

Compounds of the formula I in which R is NR¹R² and X is C₁-C₄-alkyl or C₁-C₄-haloalkyl can be obtained in an advantageous manner by the synthesis route below:

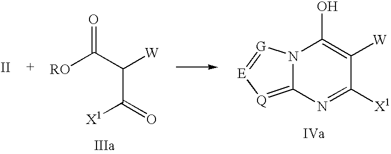

Starting with the ketoesters IIIa, the 5-alkyl-7-hydroxyazolopyrimidines IVa are obtained. In formulae IIIa and IVa, X¹ is C₁-C₄-alkyl or C₁-C₄-haloalkyl. The starting materials IIIa are advantageously prepared using the conditions described in EP-A 10 02 788 [cf. Chem. Pharm. Bull., 9, 801, (1961)].

The 5-alkyl-7-hydroxyazolopyrimidines obtained in this manner are reacted with halogenating agents [HAL] under the conditions described further above to give the 7-halo-azolopyrimidines of the formula Va in which Hal is a halogen atom. Preference is given to using chlorinating or brominating agents, such as phosphorus oxybromide, phosphorus oxychloride, thionyl chloride, thionyl bromide or sulfuryl chloride. The reaction can be carried out neat or in the presence of a solvent. Customary reaction temperatures are from 0 to 150° C. or, preferably, from 80 to 125° C.

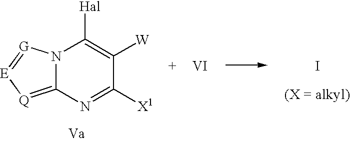

The reaction of Va with amines VI is carried out under the conditions described further above.

Alternatively, compounds of the formula I in which X is C₁-C₄-alkyl can also be prepared from compounds I in which X is halogen, in particular chlorine, and malonates of the formula IIIb. In formula IIIb, X″ is hydrogen or C₁-C₃-alkyl and R^{Π} is C₁-C₄-alkyl. They are converted into compounds of the formula VII and decarboxylated to give compounds I [cf. U.S. Pat. No. 5,994,360]. The compounds of the formula VII are novel.

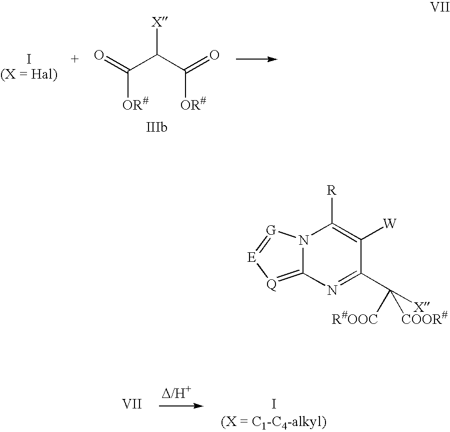

The malonates IIIb are known from the literature [J. Am. Chem. Soc., Vol. 64, 2714 (1942); J. Org. Chem., Vol. 39, 2172 (1974); Helv. Chim. Acta, Vol. 61, 1565 (1978)] or can be prepared in accordance with the literature cited.

The subsequent hydrolysis of the ester VII is carried out under generally customary conditions; depending on the various structural elements, alkaline or acidic hydrolysis of the compounds VII may be advantageous. Under the conditions of ester hydrolysis, there may already be complete or partial decarboxylation to 1.

The decarboxylation is usually carried out at temperatures of from 20° C. to 180° C., preferably from 50° C. to 120° C., in an inert solvent, if appropriate in the presence of an acid.

Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitrites, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; more preferably, the reaction is carried out in hydrochloric acid or acetic acid. It is also possible to use mixtures of the solvents mentioned.

If R in formula I is a group attached via carbon (R′ in formula Ia) and X is alkyl or haloalkyl, the compounds are prepared by reacting an aminoazole of the formula II with appropriately substituted 1,3-diketones of the formula IIIc in which R is a group attached via carbon according to formula I and X″ is alkyl or haloalkyl, preferably C₁-C₆-alkyl, in particular methyl or ethyl.

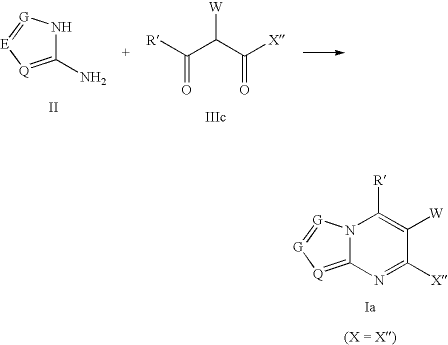

This reaction is advantageously carried out under the conditions described further above for the reaction of the compounds II with III.

Alternatively, compounds of the formula I in which R in formula I is a group attached via carbon and X is halogen, in particular chlorine, can also be prepared from dihalo compounds of the formula Va

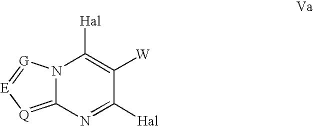

in which Hal is halogen, in particular chlorine, under the conditions known from WO 03/004465.

Compounds of the formula I in which X is cyano, alkoxy or haloalkoxy can be obtained in an advantageous manner by reacting compounds I in which X is halogen, preferably chlorine, with compounds M-X′ (formula VIII). Depending on the meaning of the group X′ to be introduced, the compound IV is an inorganic cyanide, an alkoxide or a haloalkoxide. The reaction is advantageously carried out in the presence of an inert solvent. The cation M in formula VIII is of little importance; for practical reasons, ammonium, tetraalkylammonium or alkali metal or alkaline earth metal salts are usually preferred.

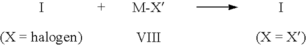

The reaction temperature is usually from 0 to 120° C., preferably from 10 to 40° C. [cf. J. Heterocycl. Chem., Vol. 12, pp. 861-863 (1975)].

Suitable solvents include ethers, such as dioxane, diethyl ether and, preferably, tetrahydrofuran, halogenated hydrocarbons, such as dichloromethane, and aromatic hydrocarbons, such as toluene.

Compounds of the formula I in which X is C₁-C₄-alkyl can also be obtained by coupling 5-haloazolopyrimidines of the formula I with organometallic reagents of the formula VIIIa. In one embodiment of this process, the reaction is carried out under transition metal catalysis, such as Ni or Pd catalysis.

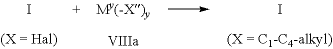

In formula VIIIa, M is a metal ion of valency Y, for example B, Zn or Sn, and X″ is C₁-C₃-alkyl. This reaction can be carried out, for example, analogously to the following methods: J. Chem. Soc. Perkin Trans. 1, 1187 (1994), ibid. 1, 2345 (1996); WO 99/41255; Aust. J. Chem., Vol. 43, 733 (1990); J. Org. Chem., Vol. 43, 358 (1978); J. Chem. Soc. Chem. Commun. 866 (1979); Tetrahedron Lett., Vol. 34, 8267 (1993); ibid., Vol. 33, 413 (1992).

Moreover, compounds of the formula I can be obtained from corresponding precursors which, instead of group P¹, bear a nucleophilically exchangeable group on group W. The group P¹ is then introduced by nucleophilic substitution [cf. WO 05/30775].

Alternatively, compounds of the formula I in which P¹ is a group attached via oxygen can be prepared from analogous hydroxyl compounds (formula IX) which for their part can be obtained by ether cleavage from known compounds [cf. WO 99/48893]. In this case, group P¹ is introduced by nucleophilic substitution of the hydroxyl group under basic conditions.

These hydroxyl compounds correspond to the formula I in which W, in addition to the Lₘ group, is substituted by a hydroxyl group (formula IX).

Compounds of the formula I in which P¹ is a group attached via nitrogen can be prepared in an advantageous manner from precursors in which the group W carries an amino group, which may be obtainable, if appropriate, from the corresponding nitro-substituted compounds by reduction.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally not necessarily required since in some cases the individual isomers can be interconverted during preparation for application or during application (for example under the action of light, acid or bases). Such conversions may also take place after application, for example in the case of the treatment of plants in the treated plant or in the harmful fungus to be controlled. In the definitions of the symbols given in the above formulae, collective terms were used which are generally representative for the following substituents:

halogen: fluorine, chlorine, bromine and iodine;

alkyl: saturated straight-chain or branched hydrocarbon radicals having 1 to 4, 6 or 8 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 2, 4 or 6 carbon atoms (as mentioned above), where some or all of the hydrogen atoms in these groups may be replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

alkenyl: unsaturated straight-chain or branched hydrocarbon radicals having 2 to 4, 6 or 8 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

alkynyl: straight-chain or branched hydrocarbon groups having 2 to 4, 6 or 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: mono- or bicyclic saturated hydrocarbon groups having 3 to 6 or 8 carbon ring members, for example C₃-C₈-cycloalkyl, such as cyclopropyl, cyclobutyl, cyclopentyl, cyclohexyl, cycloheptyl and cyclooctyl;

a five- or six-membered saturated, partially unsaturated or aromatic heterocycle which comprises one, two, three or four heteroatoms from the group consisting of O, N and S:
nonaromatic saturated or partially unsaturated 5- or 6-membered heterocyclyl which comprises one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3 dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;
5-membered heteroaryl which comprises one to four nitrogen atoms or one to three nitrogen atoms and one sulfur or oxygen atom: 5-membered heteroaryl groups which, in addition to carbon atoms, may comprise one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, and 1,3,4-triazol-2-yl;
6-membered heteroaryl which comprises one to three or one to four nitrogen atoms: 6-membered heteroaryl groups which, in addition to carbon atoms, may comprise one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl;

alkylene: divalent unbranched chains of 2 to 8 CH₂ groups, for example CH₂CH₂, CH₂CH₂CH₂, CH₂CH₂CH₂CH₂, CH₂CH₂CH₂CH₂CH₂, CH₂CH₂CH₂CH₂CH₂CH₂, CH₂CH₂CH₂CH₂CH₂CH₂CH₂ and CH₂CH₂CH₂CH₂CH₂CH₂CH₂CH₂;

oxyalkylene: divalent unbranched chains of 2 to 4 CH₂ groups where one valency is attached via an oxygen atom to the skeleton, for example OCH₂CH₂, OCH₂CH₂CH₂ and OCH₂CH₂CH₂CH₂;

oxyalkyleneoxy: divalent unbranched chains of 1 to 3 CH₂ groups where both valencies are attached via an oxygen atom to the skeleton, for example OCH₂O, OCH₂CH₂O and OCH₂CH₂CH₂O.

According to the present invention, agriculturally acceptable salts include in particular the salts of those cations or the acid addition salts of those acids whose cations and anions have no adverse effect on the pesticidal action of the inventive pyrimidines.

Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may bear from one to four (C₁-C₄)-alkyl substituents and/or one phenyl or benzyl substituent, preferably diisopropylammonium, tetramethylammonium, tetrabutylammonium, trimethylbenzylammonium, and also phosphonium ions, sulfonium ions, preferably tri(C₁-C₄)-alkylsulfonium, and sulfoxonium ions, preferably tri(C₁-C₄)-alkylsulfoxonium.

Anions of useful acid addition salts are, for example, chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and also the anions of (C₁-C₄)-alkanoic acids, preferably formate, acetate, propionate and butyrate. They can be formed by reacting the inventive compounds with an acid of the corresponding anion, preferably hydrochloric acid, hydrobromic acid, sulfuric acid, phosphoric acid or nitric acid.

The scope of the present invention includes the (R)- and (S)-isomers and the racemates of compounds of the formula I having chiral centers.

As a result of hindered rotation of asymmetrically substituted groups, atrope isomers of compounds of the formula I may be present. They also form part of the subject matter of the invention.

The embodiments of the intermediates with respect to the variables correspond to those of the formula I.

With regard to the inventive use of the azolopyrimidines of the formula I, preference is given to the following definitions of the substituents, in each case alone or in combination:

One embodiment relates to compounds I in which R is NR¹R². These compounds correspond to the formula I.a.

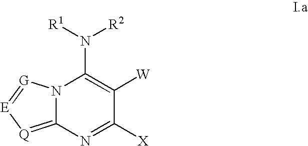

Preference is given to compounds of the formula I.a in which

R¹ is C₁-C₁₂-haloalkyl, C₂-C₁₂-haloalkenyl, C₂-C₁₂-haloalkynyl and

R² is R¹ or H, more preferably H.

Preference is further given to compounds of the formula I.a in which

R¹ is C₁-C₁₆-haloalkyl, C₂-C₆-haloalkenyl or C₂-C₆-haloalkynyl.

Preference is further given to compounds of the formula I.a in which

R¹ is C₂-haloalkyl, more preferably 2,2,2-trifluoroethyl.

Preference is further given to compounds of the formula I.a in which

R¹ is C₃-haloalkyl, more preferably 1-methyl-2,2,2-trifluoroethyl.

Preference is also given to compounds of the formula I.a in which

R¹ is C₄-C₆-haloalkyl.

Preference is given to compounds of the formula I.a in which

R² is H, C₁-C₆-alkyl, C₃-C₆-alkenyl, C₃-C₆-alkynyl, C₂-C₃-haloalkyl or C₂-C₄-haloalkenyl.

Particular preference is given to compounds in which

R² is hydrogen.

Preference is also given to compounds in which

R² is methyl.

Preference is also given to compounds in which

R² is ethyl.

Preference is also given to compounds in which

R² is propyl.

Preference is also given to compounds in which

R² is isopropyl.

Preference is also given to compounds in which

R² is allyl.

Preference is also given to compounds in which

R² is propargyl.

Particular preference is given to compounds of the formula I.a in which

R¹ is C₁-C₆-haloalkyl, C₂-C₆-haloalkenyl or C₂-C₆-haloalkynyl and

R² is R¹ or H, most preferably H.

Preference is given in particular to compounds I.a in which R¹ is an A¹ group:

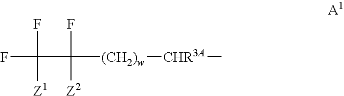

in which

Z¹ is hydrogen, fluorine or C₁-C₆-fluoroalkyl, Z² hydrogen or fluorine, or Z¹ and Z² together form a double bond; w is 0 or 1; and R^{3A} is hydrogen or methyl. R² is preferably R¹ or H, more preferably H. Methyl or ethyl, especially preferably H.

Z¹ and Z² are each independently preferably fluorine or hydrogen or form a double bond.

If R¹ and/or R² includes haloalkyl or haloalkenyl groups with a center of chirality, preference is given to the (S)-isomers for these groups. In the case of halogen-free alkyl or alkenyl groups with centers of chirality in R¹ or R², preference is given to the (R)-configured isomers.

A further embodiment relates to compounds I in which R is a group bonded via carbon. These compounds correspond to the formula I.b in which R′ is C₃-C₆-cycloalkyl or C₃-C₁₂-halocycloalkyl.

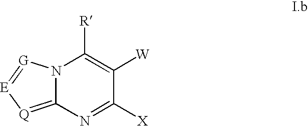

A further embodiment relates to compounds I.b in which R′ is C₃-C₈-halocycloalkyl or C₃-C₆-cycloalkyl.

A preferred embodiment relates to compounds I.b in which R′ is C₃-cycloalkyl, C₅- or C₆-cycloalkyl, especially C₆-cycloalkyl.

A further preferred embodiment relates to compounds I.b in which R′ is C₃-C₆-cycloalkyl, more preferably C₆-cycloalkyl, or C₃-C₆-halocycloalkyl.

A further embodiment relates to compounds I.b in which R′ is C₃-halocycloalkyl, C₄—, C₅- or C₆-halocycloalkyl.

If R′ bears at least one R^{b} group, R^{b} is preferably selected from halogen, cyano, C₁-C₆-alkyl, C₁-C₆-haloalkyl, C₂-C₆-alkenyl, C₂-C₆-alkynyl, C₁-C₆-alkylcarbonyl, C₁-C₆-haloalkylcarbonyl and C₁-C₆-alkoxy.

One embodiment relates to compounds I in which W is phenyl substituted by P¹ and Lₘ.

Useful groups for Lₘ are in particular the following groups: halogen, such as fluorine or chlorine; cyano; nitro; alkoxycarbonyl; aminocarbonyl; C₁-C₄-alkyl, such as methyl; C₁-C₄-haloalkyl, such as trifluoromethyl; C₁-C₄-alkoxy, such as methoxy.

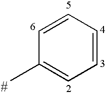

Embodiments of the W group relate in particular to phenyl groups which, in addition to the P¹ group, may have the following substitution:

position 2: fluorine, chlorine or methyl; position 3: hydrogen, fluorine or methoxy; position 4: hydrogen, fluorine, chlorine, methyl, methoxy, cyano, nitro, alkoxycarbonyl, aminocarbonyl or haloalkyl, more preferably fluorine, chlorine, methyl, methoxy or cyano; position 5: hydrogen, fluorine, chlorine or methyl; more preferably hydrogen or fluorine; position 6: hydrogen, fluorine, chlorine or methyl; more preferably hydrogen or fluorine.

The P¹ group is preferably located in the 3, 4 or 5 positions.

In two embodiments of the compounds I, the phenyl group substituted by the P¹ and Lₘ groups is the A or B group.

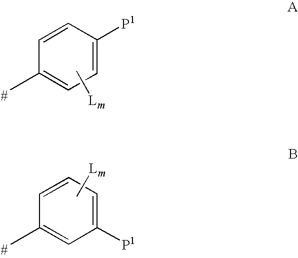

In a further embodiment of the compounds I, in particular in the A and B groups, Lₘ is one of the following combinations of substituents: 2-Cl; 2-F; 2,6-Cl₂; 2,6-F₂; 2-F, 6-Cl; 2-F, 6-CH₃; 2,4,6-F₃; 2,6-F₂-4-OCH₃; 2-C₁₋₄—OCH₃; 2-F, 4-OCH₃, 2-CH₃, 4-OCH₃, 2-CH₃-4-F; 2-CF₃; 2-OCH₃, 6-F; 2,4-F₂; 2-F-4-Cl; 2-Cl, 4-F; 2-Cl, 5-F; 2,3-F₂; 2,5-F₂; 2,3,4-F₃; 2-CH₃; 2,4-(CH₃)₂; 2-CH₃-4-Cl; 2-CH₃, 5-F; 2-F, 4-CH₃; 2,6-(CH₃)₂; 2,4,6-(CH₃)₃; 2,6-F₂, 4-CH₃.

In a further embodiment of the compounds I, in particular in the A group, Lₘ is one of the following combinations of substituents: 2-F; 2-Cl; 2-CH₃; 2,6-F₂; 2-F, 6-Cl; 2-F, 6-CH₃.

The compounds of the formula I which bear the A or B group correspond to formulae I.A and I.B respectively.

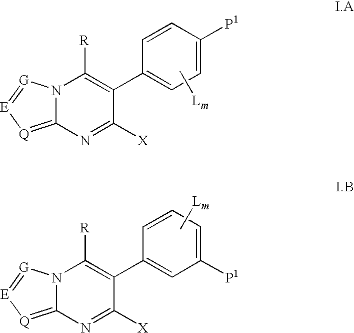

One embodiment relates to compounds I in which W is heteroaryl which is substituted by P¹ and Lₘ and comprises one to four nitrogen atoms or one to three nitrogen atoms and one sulfur or oxygen atom.

In one embodiment, the W group is heteroaryl which is substituted by P¹ and Lₘ and attached via a nitrogen atom.

In a further embodiment, the W group is heteroaryl which is substituted by P¹ and Lₘ and attached via a carbon atom.

One embodiment relates to compounds I in which W is a 5-membered heteroaryl which is substituted by P¹ and Lₘ and comprises one to four nitrogen atoms or one to three nitrogen atoms and one sulfur or oxygen atom.

A further embodiment relates to compounds I in which W is pyrrole, furan, thiophene, pyrazole, isoxazole, isothiazole, imidazole, oxazole, thiazole, 1,2,3-triazole or 1,2,4-triazole.

A further embodiment relates to compounds I in which W is a thiophene, pyrazole or thiazole.

One embodiment relates to compounds I in which W is 6-membered heteroaryl which is substituted by P¹ and Lₘ and comprises one to three or one to four nitrogen atoms.

A further embodiment relates to compounds I in which W is pyridine, pyrimidine, pyridazine or pyrazine.

One embodiment relates to compounds I in which W is pyridyl which is attached in the 2-, 3- or 4-position and which may be mono- to tetrasubstituted by identical or different Lₘ, which is preferably fluorine, chlorine, bromine, cyano, nitro, methyl, ethyl, methoxy, methylthio, hydroximinomethyl, hydroximinoethyl, methoximinomethyl, methoximinoethyl and/or trifluoromethyl, more preferably fluorine, chlorine, methyl, methoxy and/or cyano.

One embodiment of the compounds of the formula I relates to those of the formulae I.C and I.D.

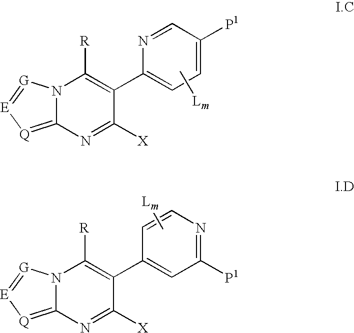

A further embodiment relates to compounds I in which W is pyrimidyl which is attached in the 2- or 4-position and may be mono- or disubstituted by identical or different Lₘ, which here is preferably fluorine, chlorine, bromine, cyano, nitro, methyl, ethyl, methoxy, methylthio, hydroximinomethyl, hydroximinoethyl, methoximinomethyl, methoximinoethyl and/or trifluoromethyl, more preferably fluorine, chlorine, methyl, methoxy and/or cyano.

One embodiment of the compounds of the formula I relates to those of the formulae I.E and I.F.

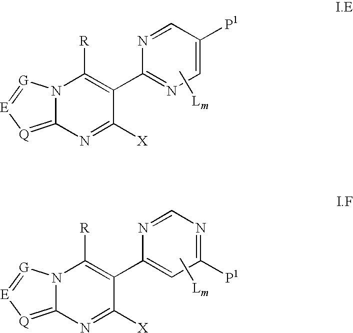

A further embodiment relates to compounds I in which W is thienyl which is attached in the 2- or 3-position and may be mono- or disubstituted by identical or different Lₘ, which is preferably fluorine, chlorine, bromine, cyano, nitro, methyl, ethyl, methoxy, methylthio, hydroximinomethyl, hydroximinoethyl, methoximinomethyl, methoximinoethyl and/or trifluoromethyl, more preferably fluorine, chlorine, methyl, methoxy and/or cyano.

One embodiment of the compounds of the formula I relates to those of the formulae I.G and I.H.

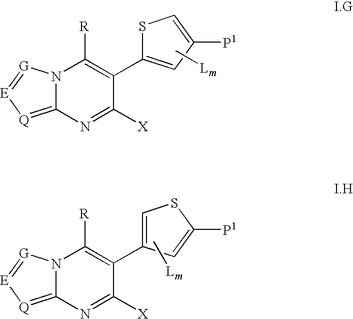

A further embodiment relates to compounds I in which W is thiazolyl which is attached in the 2-, 4- or 5-position and may be substituted by Lₘ, which here is preferably fluorine, chlorine, bromine, cyano, nitro, methyl, ethyl, methoxy, methylthio, hydroximinomethyl, hydroximinoethyl, methoximinomethyl, methoximinoethyl or trifluoromethyl, more preferably fluorine, chlorine, methyl, methoxy and/or cyano.

One embodiment of the compounds of the formula I relates to those of the formulae I.I and I.J.

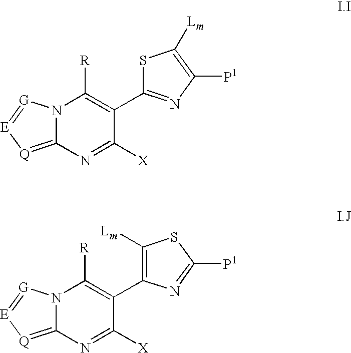

A further embodiment relates to compounds I in which W is imidazolyl which is attached in the 4- or 5-position and may be mono- or disubstituted by identical or different Lₘ, which here is preferably fluorine, chlorine, bromine, cyano, nitro, methyl, ethyl, methoxy, methylthio, hydroximinomethyl, hydroximinoethyl, methoximinomethyl, methoximinoethyl and/or trifluoromethyl, more preferably fluorine, chlorine, methyl, methoxy and/or cyano.

One embodiment of the compounds of the formula I relates to those of the formulae I.K and I.L.

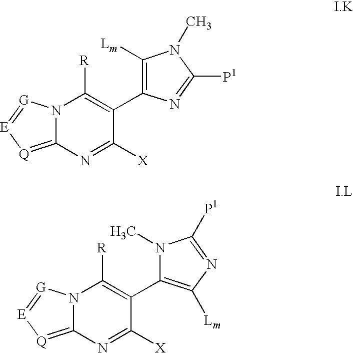

A further embodiment relates to compounds I in which W is pyrazolyl which is attached in the 1-, 3-, 4- or 5-position and may be mono- to trisubstituted by identical or different Lₘ, which here is preferably fluorine, chlorine, bromine, cyano, nitro, methyl, ethyl, methoxy, methylthio, hydroximinomethyl, hydroximinoethyl, methoximinomethyl, methoximinoethyl and/or trifluoromethyl, more preferably fluorine, chlorine, methyl, methoxy and/or cyano.

One embodiment of the compounds of the formula I relates to those of the formulae I.M, I.N and I.O.

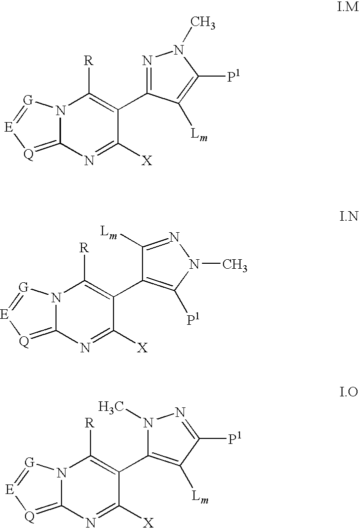

A further embodiment relates to compounds I in which W is oxazolyl which is attached in the 2-, 3- or 4-position and may be mono- or disubstituted by identical or different Lₘ, which here is preferably fluorine, chlorine, bromine, cyano, nitro, methyl, ethyl, methoxy, methylthio, hydroximinomethyl, hydroximinoethyl, methoximinomethyl, methoximinoethyl and/or trifluoromethyl, more preferably fluorine, chlorine, methyl, methoxy and/or cyano.

One embodiment of the compounds of the formula I relates to those of the formulae I.P and I.Q

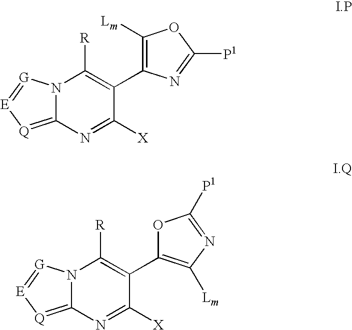

In a preferred embodiment of the compounds I, in particular the formulae I.A to I.Q, at least one group L is located ortho to the point of attachment of the W group to the azolopyrimidine skeleton, in particular chlorine, fluorine or methyl.

In a further embodiment, a heteroatom of the heteroaromatic radical W is located ortho to the point of attachment.

The index m is, if structurally possible, preferably 1 to 4, where the groups L may be identical or different. If the heteroaromatic groups W carry, in addition to a P¹ group, further substituents, these are preferably selected from the group consisting of: fluorine, chlorine, methyl, methoxy, cyano, nitro, alkoxycarbonyl, aminocarbonyl and haloalkyl. In a further embodiment, the optional substituents Lₘ are selected from the group consisting of fluorine, chlorine, methyl and methoxy. In a further embodiment, the optional substituents Lₘ are selected from the group consisting of chlorine, methyl and methoxy. A further embodiment relates to heteroaromatic groups W which, in addition to a P¹ group, are substituted by chlorine. A further embodiment relates to heteroaromatic W groups which, in addition to a P¹ group, are substituted by fluorine.

In one embodiment of the P¹ group, Y¹ is CR^{a}R^{a′}.

In a further embodiment of the P¹ group, Y¹ is C(O)O.

In a further embodiment of the P¹ group, Y¹ is C(O)NR^{b}.

In a further embodiment of the P¹ group, Y¹ is oxygen.

In a further embodiment of the P¹ group, Y¹ is NR^{b}.

In a further embodiment of the P¹ group, Y¹ is sulfur.

In one embodiment of the P¹ group, Y² is C₁-C₈-alkylene, preferably C₂-C₈-alkylene.

In a further embodiment of the P¹ group, Y² is C₃-C₈-alkylene, preferably C₃-C₄-alkylene, more preferably C₃-alkylene (propylene).

In a further embodiment of the P¹ group, Y² is C₁-alkylene (methylene).

In a further embodiment of the P¹ group, Y² is C₂-alkylene (ethylene).

In a further embodiment of the P¹ group, Y² is ##-CH(CH₃)—CH₂— (## is the point of attachment to Y¹).

In a further embodiment of the P¹ group, Y² is ##-CH₂—CH(CH₃)— (## is the point of attachment to Y¹).

In a further embodiment of the P¹ group, Y² is C₄-alkylene (butylene).

In a further embodiment of the P¹ group, Y² is C₂-C₈-alkenylene.

In a further embodiment of the P¹ group, Y² is C₂-C₈-alkynylene.

In a further embodiment of the P¹ group, Y², in particular C₁-C₈-alkylene, is interrupted by heteroatoms. Suitable heteroatoms are in particular oxygen and NR^{b}, where R^{b} in this case is preferably hydrogen, C₁-C₄-alkylcarbonyl, C₁-C₄-alkoxycarbonyl or C₁-C₄-alkyl, preferably methyl.

One embodiment relates to compounds I in which T is OH.

A further embodiment relates to compounds I in which T is OR^{c} where R^{c} is preferably:
hydrogen, cyano, C₁-C₆-alkyl, C₂-C₆-alkenyl, C₂-C₆-alkynyl, C₃-C₆-cycloalkyl, C₃-C₈-cycloalkenyl, C(O)R^{c}, C(O)OR^{c}, C(S)OR^{c}, C(O)SR^{r}, C(S)SR^{r}, amino, C₁-C₆-alkylamino, di-C₁-C₆-alkylamino, aminocarbonyl, C(O)NHR^{Π}, C(O)NR^{Π}₂, C₁-C₆-alkylene, phenyl, naphthyl, five-, six-, seven-, eight-, nine- or ten-membered saturated, partially unsaturated or aromatic heterocycle comprising one, two, three or four heteroatoms from the group of O, N and S;

A further embodiment relates to compounds I in which T is OR where R^{c} is preferably C₁-C₄-alkyl, more preferably methyl.

A further embodiment relates to compounds I in which T is OR^{c} in which R^{c} is C₃-C₆-cycloalkyl; 5- or 6-membered heterocyclyl or 5- or 6-membered heteroaryl, preferably 6-membered hetaryl such as pyridine, pyridazine, pyrimidine, pyrazine, 1,2,4-triazine and 1,3,5-triazine, and also preferably 5-membered heteroaryl such as pyrazole, isoxazole, isothiazole, imidazole, thiazole and oxazole, and also preferably 6-membered heterocyclyl such as tetrahydropyran and piperidine, and also preferably 5-membered heterocyclyl such as tetrahydrofuran and pyrrolidine.

In a further embodiment, the R^{a} and R^{Π} radicals specified as the definition for T is OR^{c} are unsubstituted or substituted by 1-3 substituents R^{d} where R^{d} is preferably defined as follows: halogen, cyano, nitro, hydroxyl, mercapto, amino, carboxyl, alkyl, haloalkyl, alkenyl, alkynyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoxyimino (═N—O—C₁-C₈-alkyl), C₃-C₈-alkenyloximino (═N—O—C₃-C₈-alkenyl) or C₃-C₈-alkynyloximino (═N—O—C₃-C₈-alkynyl). Preference is also given to halogen, hydroxyl, amino, carboxyl, alkyl, haloalkyl, alkoxy, haloalkoxy, alkylthio, alkylamino, dialkylamino, alkylsulfonyl, alkylsulfoxyl, alkoxycarbonyl, alkylcarbonyloxy, alkoxycarbonyloxy, aminocarbonyl, alkylaminocarbonyl, dialkylaminocarbonyl, oxo (═O), thioxo (═S), C₁-C₈-alkoximino (═N—O—C₁-C₈-alkyl), C₃-C₈-alkenyloximino (═N—O—C₃-C₈-alkenyl) or C₃-C₈-alkynyloximino (═N—O—C₃-C₈-alkynyl), especially halogen, hydroxyl, amino, alkyl, haloalkyl, alkoxy, alkylthio, alkylamino, dialkylamino, alkoxycarbonyl, alkylcarbonyloxy, alkoxycarbonyloxy, aminocarbonyl, alkylaminocarbonyl, dialkylaminocarbonyl or oxo (═O), more preferably halogen, alkyl, alkoxy or oxo (═O), where the alkyl groups in these radicals comprise from 1 to 6 carbon atoms and the alkenyl and alkynyl groups in these radicals comprise from 2 to 8 carbon atoms.
A further embodiment relates to compounds I in which T is OC(O)R^{a} where R^{a} is defined as follows: hydrogen, cyano, carboxyl, C₁-C₆-alkyl, C₂-C₆-alkenyl, C₂-C₆-alkynyl, C₃-C₆-cycloalkyl, C₃-C₈-cycloalkenyl, C₁-C₆-alkoxy, C₂-C₆-alkenyloxy, C₃-C₆-alkynyloxy, C₃-C₆-cycloalkoxy, C₃-C₆-cycloalkenyloxy, C(O)R^{Π}, C(O)OR^{Π}, C(S)OR^{Π}, C(O)SR^{Π}, C(S)SR^{Π}, OC(O)OR^{Π}, C₁-C₆-alkylthio, amino, C₁-C₆-alkylamino, di-C₁-C₆-alkylamino, aminocarbonyl, C(O)NHR^{Π}, C(O)NR^{Π}, phenyl, naphthyl, five-, six-, seven-, eight-, nine- or ten-membered saturated, partially unsaturated or aromatic heterocycle comprising one, two, three or four heteroatoms from the group of O, N and S. R^{a} is preferably hydrogen, C₁-C₆-alkyl, C₁-C₆-alkoxy, C(O)R^{Π}, C(O)OR^{Π}, amino, C₁-C₆-alkylamino, di-C₁-C₆-alkylamino, aminocarbonyl, C(O)NHR^{Π} or C(O)NR^{Π}.
   In a further embodiment, the R^{a} and R^{Π} radicals specified as the definition for T is OC(O)R^{a} are unsubstituted or substituted by 1-3 substituents R^{d} where R^{d} is preferably defined as follows: halogen, cyano, nitro, hydroxyl, mercapto, amino, carboxyl, alkyl, haloalkyl, alkenyl, alkynyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl) or C₃-C₈-alkynyloximino (═N—O—C₃-C₈-alkynyl), more preferably halogen, alkyl, alkoxy or oxo (═O), where the alkyl groups in these radicals comprise from 1 to 6 carbon atoms and the alkenyl or alkynyl groups in these radicals comprise from 2 to 8 carbon atoms.

A further embodiment relates to compounds I in which T is NR^{b}R^{b′} where R^{b′} may be as defined for R^{b}, and R^{b} and R^{b′} are each independently
hydrogen, cyano, nitro, hydroxyl, carboxyl, C₁-C₆-alkyl, C₂-C₆-alkenyl, C₂-C₆-alkynyl, C₃-C₆-cycloalkyl, C₃-C₈-cycloalkenyl, C₁-C₆-alkoxy, C₂-C₆-alkenyloxy, C₃-C₆-alkynyloxy, C₃-C₆-cycloalkoxy, C₃-C₆-cycloalkenyloxy, C(O)R^{Π}, C(O)OR^{Π}, C(S)OR^{Π}, C(O)SR^{Π}, C(S)SR^{Π}, OC(O)OR^{Π}, C₁-C₆-alkylthio, amino, C₁-C₆-alkylamino, di-C₁-C₆-alkylamino, aminocarbonyl, C(O)NHR^{Π}, C(O)NR^{Π}₂, phenyl, naphthyl, five-, six-, seven-, eight-, nine- or ten-membered saturated, partially unsaturated or aromatic heterocycle comprising one, two, three or four heteroatoms from the group of O, N and S.

In a further embodiment of T=NR^{b}R^{b′}, R^{b} and R^{b′} are preferably hydrogen, cyano, C₁-C₆-alkyl, C₂-C₆-alkenyl, C₂-C₆-alkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, amino, C₁-C₆-alkylamino, di-C₁-C₆-alkylamino, aminocarbonyl, C(O)NHR^{Π} or C(O)NR^{Π}₂.

In a further embodiment of T=NR^{b}R^{b′}, R^{b} and R^{b} are preferably hydrogen, C₁-C₆-alkyl, C(O)R^{Π}, C(O)OR^{Π}, aminocarbonyl, C(O)NHR^{Π} or C(O)NR^{Π}₂.

In a further embodiment of T=NR^{b}R^{b′}, R^{b} and R^{b′}, together with the nitrogen to which they are bonded, form a five-, six-, seven-, eight-, nine- or ten-membered saturated, partially unsaturated or aromatic heterocycle comprising one, two, three or four heteroatoms from the group of O, N and S.

In a further embodiment, the R^{b}, R^{b′} and R^{Π} radicals specified as a definition for T=NR^{b}R^{b′} are unsubstituted or substituted by 1-3 substituents R^{d} where R^{d} is preferably defined as follows: halogen, cyano, nitro, hydroxyl, mercapto, amino, carboxyl, alkyl, haloalkyl, alkenyl, alkynyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl) or C₃-C₈-alkynyloximino (═N—O—C₃-C₈-alkynyl). Preference is also given to halogen, hydroxyl, amino, carboxyl, alkyl, haloalkyl, alkoxy, haloalkoxy, alkylthio, alkylamino, dialkylamino, alkylsulfonyl, alkylsulfoxyl, alkoxycarbonyl, alkylcarbonyloxy, alkoxycarbonyloxy, aminocarbonyl, alkylaminocarbonyl, dialkylaminocarbonyl, oxo (═O), thioxo (═S), C₁-C₈-alkoximino (═N—O—C₁-C₈-alkyl), C₃-C₈-alkenyloximino (═N—O—C₃-C₈-alkenyl) or C₃-C₈-alkynyloximino (═N—O—C₃-C₈-alkynyl), especially halogen, hydroxyl, amino, alkyl, haloalkyl, alkoxy, alkylthio, alkylamino, dialkylamino, alkoxycarbonyl, alkylcarbonyloxy, alkoxycarbonyloxy, aminocarbonyl, alkylaminocarbonyl, dialkylaminocarbonyl or oxo (═O), more preferably halogen, alkyl, alkoxy or oxo (═O), where the alkyl groups in these radicals comprise from 1 to 6 carbon atoms and the alkenyl and alkynyl groups in these radicals comprise from 2 to 8 carbon atoms.

Preferred definitions for NR^{b}R^{b′} are amino, methylamino, dimethylamino, pyrrolidinyl, piperidinyl, piperazinyl, N-methylpiperazinyl, morpholinyl, pyrazolyl, triazinyl and pyrrolidonyl, more preferably methylamino, dimethylamino, piperazinyl and N-methylpiperazinyl.

In one embodiment, the N^{b}R^{a′} group is dimethylamino.

In a further embodiment, the group is methylamino.

In a further embodiment, the group is amino.

A further embodiment relates to compounds I in which T is C(O)NR^{b}R^{b′}, where R^{b′} may be as defined for R^{b}, and R^{b} and R^{b′} are each independently
hydrogen, cyano, nitro, hydroxyl, carboxyl, C₁-C₆-alkyl, C₂-C₆-alkenyl, C₂-C₆-alkynyl, C₃-C₆-cycloalkyl, C₃-C₈-cycloalkenyl, C₁-C₆-alkoxy, C₂-C₆-alkenyloxy, C₃-C₆-alkynyloxy, C₃-C₆-cycloalkoxy, C₃-C₆-cycloalkenyloxy, C(O)R^{Π}, C(O)OR^{Π}, C(S)OR^{Π}, C(O)SR^{Π}, C(S)SR^{Π}, OC(O)OR⁹⁰, C₁-C₆-alkylthio, amino, C₁-C₆-alkylamino, di-C₁-C₆-alkylamino, aminocarbonyl, C(O)NHR^{Π}, C(O)NR^{Π}₂, phenyl, naphthyl, five-, six-, seven-, eight-, nine- or ten-membered saturated, partially unsaturated or aromatic heterocycle comprising one, two, three or four heteroatoms from the group of O, N and S.

In a further embodiment of T=C(O)NR^{b}R^{b′}, R^{b} and R^{b′} are preferably hydrogen, cyano, C₁-C₆-alkyl, C₂-C₆-alkenyl, C₂-C₆-alkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, amino, C₁-C₆-alkylamino, di-C₁-C₆-alkylamino, aminocarbonyl, C(O)NHR^{Π} or C(O)NR^{Π}₂.

In a further embodiment of T=C(O)NR^{b}R^{b′}, R^{b} and R^{b′} are preferably hydrogen, C₁-C₆-alkyl, C(O)R^{Π}, C(O)OR^{Π}, aminocarbonyl, C(O)NHR^{Π} or C(O)NR^{Π}₂.

In a further embodiment of T=C(O)NR^{b}R^{b′}, R^{b} and R^{b′}, together with the nitrogen to which they are bonded, form a five-, six-, seven-, eight-, nine- or ten-membered saturated, partially unsaturated or aromatic heterocycle comprising one, two, three or four heteroatoms from the group of O, N and S.

In a further embodiment, the R^{b}, R^{b′} and R^{Π} radicals specified as a definition for T=C(O)NR^{b}R^{b′} are unsubstituted or substituted by 1-3 substituents R^{d} where R^{d} is preferably defined as follows: halogen, cyano, hydroxyl, mercapto, amino, carboxyl, alkyl, haloalkyl, alkenyl, alkynyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl) or C₃-C₈-alkynyloximino (═N—O—C₃-C₈-alkynyl). Preference is also given to halogen, hydroxyl, amino, carboxyl, alkyl, haloalkyl, alkoxy, haloalkoxy, alkylthio, alkylamino, dialkylamino, alkylsulfonyl, alkylsulfoxyl, alkoxycarbonyl, alkylcarbonyloxy, alkoxycarbonyloxy, aminocarbonyl, alkylaminocarbonyl, dialkylaminocarbonyl, oxo (═O), thioxo (═S), C₁-C₈-alkoximino (═N—O—C₁-C₈-alkyl), C₃-C₈-alkenyloximino (═N—O—C₃-C₈-alkenyl) or C₃-C₈-alkynyloximino (═N—O—C₃-C₈-alkynyl), especially preferably halogen, hydroxyl, amino, alkyl, haloalkyl, alkoxy, alkylthio, alkylamino, dialkylamino, alkoxycarbonyl, alkylcarbonyloxy, alkoxycarbonyloxy, aminocarbonyl, alkylaminocarbonyl, dialkylaminocarbonyl or oxo (═O), more preferably halogen, alkyl, alkoxy or oxo (═O), where the alkyl groups in these radicals comprise from 1 to 6 carbon atoms and the alkenyl or alkynyl groups in these radicals comprise from 2 to 8 carbon atoms.

A further embodiment relates to compounds I in which T is C(═NOR^{c})R^{a} in which R^{a} is hydrogen, cyano, hydroxyl, carboxyl, C₁-C₆-alkyl, C₂-C₆-alkenyl, C₂-C₆-alkynyl, C₃-C₆-cycloalkyl, C₃-C₈-cycloalkenyl, C₁-C₆-alkoxy, C₂-C₆-alkenyloxy, C₃-C₆-alkynyloxy, C₃-C₆-cycloalkoxy, C₃-C₆-cycloalkenyloxy, C(O)R^{Π}, C(O)OR^{Π}, C(S)OR^{Π}, C(O)SR^{Π}, C(S)SR^{Π}, OC(O)OR^{Π}, C₁-C₆-alkylthio, amino, C₁-C₆-alkylamino, di-C₁-C₆-alkylamino, aminocarbonyl, C(O)NHR^{Π}, C(O)NR^{Π}₂, phenyl, naphthyl, five-, six-, seven-, eight-, nine- or ten-membered saturated, partially unsaturated or aromatic heterocycle comprising one, two, three or four heteroatoms from the group of O, N and S, and R^{c} is hydrogen, cyano, C₁-C₆-alkyl, C₂-C₆-alkenyl, C₂-C₆-alkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, OC(O)OR^{Π}, amino, C₁-C₆-alkylamino, di-C₁-C₆-alkylamino, aminocarbonyl, C(O)NHR^{Π}, C(O)NR^{Π}₂, phenyl, naphthyl, five-, six-, seven-, eight-, nine- or ten-membered saturated, partially unsaturated or aromatic heterocycle comprising one, two, three or four heteroatoms from the group of O, N and S.

In a further embodiment of T=C(═NOR^{c})R^{a}, R^{c} is preferably hydrogen, C₁-C₆-alkyl, C₂-C₆-alkenyl, C₂-C₆-alkynyl, C₃-C₆-cycloalkyl, C₃-C₈-cycloalkenyl, C(O)R^{Π}, C(O)OR^{Π}, aminocarbonyl, C(O)NHR^{Π} or C(O)NR^{Π}₂, more preferably hydrogen, C₁-C₆-alkyl, C₂-C₆-alkenyl or C₂-C₆-alkynyl.

In a further embodiment of T=C(═NOR^{c})R^{a}, R^{c} is preferably

hydrogen, hydroxyl, C₁-C₆-alkyl, C₂-C₆-alkenyl, C₂-C₆-alkynyl, C₃-C₆-cycloalkyl, C₃-C₈-cycloalkenyl, C₁-C₆-alkoxy, C₂-C₆-alkenyloxy, C₃-C₆-alkynyloxy, C₃-C₆-cycloalkoxy, C₃-C₆-cycloalkenyloxy, amino, C₁-C₆-alkylamino or di-C₁-C₆-alkylamino, more preferably hydrogen, hydroxyl, C₁-C₆-alkyl, C₁-C₆-alkoxy, amino, C₁-C₆-alkylamino or di-C₁-C₆-alkylamino.

In a further embodiment, the R^{a}, R^{c} and R^{Π} radicals specified as a definition for T=C(═NOR^{c})R^{a} are unsubstituted or substituted by 1-3 substituents R^{d} where R^{d} is preferably defined as follows: halogen, cyano, nitro, hydroxyl, mercapto, amino, carboxyl, alkyl, haloalkyl, alkenyl, alkynyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl) or C₃-C₈-alkynyloximino (═N—O—C₃-C₈-alkynyl). Preference is also given to halogen, hydroxyl, amino, carboxyl, alkyl, haloalkyl, alkoxy, haloalkoxy, alkylthio, alkylamino, dialkylamino, alkylsulfonyl, alkylsulfoxyl, alkoxycarbonyl, alkylcarbonyloxy, alkoxycarbonyloxy, aminocarbonyl, alkylaminocarbonyl, dialkylaminocarbonyl, oxo (═O), thioxo (═S), C₁-C₈-alkoximino (═N—O—C₁-C₈-alkyl), C₃-C₈-alkenyloximino (═N—O—C₃-C₈-alkenyl) or C₃-C₈-alkynyloximino (═N—O—C₃-C₈-alkynyl), especially halogen, hydroxyl, amino, alkyl, haloalkyl, alkoxy, alkylthio, alkylamino, dialkylamino, alkoxycarbonyl, alkylcarbonyloxy, alkoxycarbonyloxy, aminocarbonyl, alkylaminocarbonyl, dialkylaminocarbonyl or oxo (═O), more preferably halogen, alkyl, alkoxy or oxo (═O), where the alkyl groups in these radicals comprise from 1 to 6 carbon atoms and the alkenyl or alkynyl groups in these radicals comprise from 2 to 8 carbon atoms.

A further embodiment relates to compounds I in which T is T¹-C(=T²)T³.

In a further embodiment, T¹ is oxygen.

In a further embodiment, T¹ is NR^{b}.

In a further embodiment, T² is oxygen.

In a further embodiment, T² is sulfur.

In a further embodiment, T² is NR^{b}.

In a further embodiment, T³ is R^{a}.

In a further embodiment, T³ is OR^{c}.

In a further embodiment, T³ is SR^{c}.

In a further embodiment, T³ is NR^{b}R^{b′}.

In a further embodiment of T¹-C(=T²)T³, R^{a} is preferably

hydrogen, cyano, hydroxyl, carboxyl, C₁-C₆-alkyl, C₂-C₆-alkenyl, C₂-C₆-alkynyl, C₃-C₆-cycloalkyl, C₃-C₈-cycloalkenyl, C₁-C₆-alkoxy, C₂-C₆-alkenyloxy, C₃-C₆-alkynyloxy, C₃-C₆-cycloalkoxy, C₃-C₆-cycloalkenyloxy, C(O)R^{Π}, C(O)OR^{Π}, C(S)OR^{Π}, C(O)SR^{Π}, C(S)SR^{Π}, OC(O)OR^{Π}, C₁-C₆-alkylthio, amino, C₁-C₆-alkylamino, di-C₁-C₆-alkylamino, aminocarbonyl, C(O)NHR^{Π}, C(O)NR^{Π}₂, phenyl, naphthyl, five-, six-, seven-, eight-, nine- or ten-membered saturated, partially unsaturated or aromatic heterocycle comprising one, two, three or four heteroatoms from the group of O, N and S.

In a further embodiment of T¹-C(=T²)T³, R^{b} and R^{c} are preferably hydrogen, C₁-C₆-alkyl, C₂-C₆-alkenyl, C₂-C₆-alkynyl, C₃-C₆-cycloalkyl, C₃-C₈-cycloalkenyl, C(O)R^{Π}, C(O)OR^{Π}, aminocarbonyl, C(O)NHR^{Π} or C(O)NR^{Π}₂.

In a further embodiment, the R^{a}, R^{b}, R^{c} and R^{Π} radicals mentioned as a definition for T¹-C(=T²)T³ are unsubstituted or substituted by 1-3 substituents R^{d} where R^{d} is preferably defined as follows: halogen, cyano, nitro, hydroxyl, mercapto, amino, carboxyl, alkyl, haloalkyl, alkenyl, alkynyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl) or C₃-C₈-alkynyloximino (═N—O—C₃-C₈-alkynyl). Preference is also given to halogen, hydroxyl, amino, carboxyl, alkyl, haloalkyl, alkoxy, haloalkoxy, alkylthio, alkylamino, dialkylamino, alkylsulfonyl, alkylsulfoxyl, alkoxycarbonyl, alkylcarbonyloxy, alkoxycarbonyloxy, aminocarbonyl, alkylaminocarbonyl, dialkylaminocarbonyl, oxo (═O), thioxo (═S), C₁-C₈-alkoximino (═N—O—C₁-C₈-alkyl), C₃-C₈-alkenyloximino (═N—O—C₃-C₈-alkenyl) or C₃-C₈-alkynyloximino (═N—O—C₃-C₈-alkynyl), especially halogen, hydroxyl, amino, alkyl, haloalkyl, alkoxy, alkylthio, alkylamino, dialkylamino, alkoxycarbonyl, alkylcarbonyloxy, alkoxycarbonyloxy, aminocarbonyl, alkylaminocarbonyl, dialkylaminocarbonyl or oxo (═O), more preferably halogen, alkyl, alkoxy or oxo (═O), where the alkyl groups in these radicals comprise from 1 to 6 carbon atoms and the alkenyl or alkynyl groups in these radicals comprise from 2 to 8 carbon atoms.

One embodiment relates to compounds I in which X is fluorine, chlorine or bromine, especially chlorine.

A further embodiment relates to compounds I in which X is fluorine.

A further embodiment relates to compounds I in which X is cyano.

A further embodiment relates to compounds I in which X alkyl, especially methyl.

A further embodiment relates to compounds I in which X alkoxy, especially methoxy.

One embodiment relates to compounds I in which G is N; E is C—W² and Q is N. These compounds correspond to formula I.1.

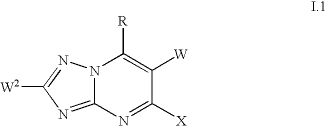

A further embodiment relates to compounds I in which G is N; E is C—W² and Q is C—W³. These compounds correspond to formula I.2.

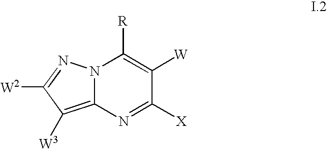

A further embodiment relates to compounds I in which G is C—W¹; E is C—W² and Q is N. These compounds correspond to formula I.3.

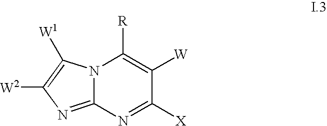

A further embodiment relates to compounds I in which G is C—W¹; E is N and Q is C—W³. These compounds correspond to formula I.4.

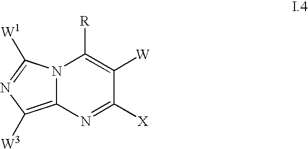

In one embodiment of the compounds I, W¹ is hydrogen, fluorine, chlorine or bromine, in particular hydrogen.

In one embodiment of the compounds I, W² is hydrogen, cyano, fluorine, chlorine, bromine, iodine, nitro, formyl, haloalkyl having from 1 to 4 carbon atoms and from 1 to 9 fluorine, chlorine and/or bromine atoms, alkyl having from 1 to 4 carbon atoms, cycloalkyl having from 3 to 6 carbon atoms, thiocarbamoyl, alkoxycarbonyl having from 1 to 4 carbon atoms in the alkoxy moiety, alkylcarbonyl having from 1 to 4 carbon atoms in the alkyl moiety, hydroximinoalkyl having from 1 to 4 carbon atoms in the alkyl moiety or is alkoxyiminoalkyl having from 1 to 4 carbon atoms in the alkoxy moiety and from 1 to 4 carbon atoms in the alkyl moiety, in particular hydrogen, amino or C₁-C₄-alkyl, preferably hydrogen.

In one embodiment of the compounds I, W³ is hydrogen, cyano, fluorine, chlorine, bromine, iodine, nitro, formyl, haloalkyl having from 1 to 4 carbon atoms and from 1 to 9 fluorine, chlorine and/or bromine atoms, alkyl having from 1 to 4 carbon atoms, cycloalkyl having from 3 to 6 carbon atoms, thiocarbamoyl, alkoxycarbonyl having from 1 to 4 carbon atoms in the alkoxy moiety, alkylcarbonyl having from 1 to 4 carbon atoms in the alkyl moiety, hydroximinoalkyl having from 1 to 4 carbon atoms in the alkyl moiety or is alkoxyiminoalkyl having from 1 to 4 carbon atoms in the alkoxy moiety and from 1 to 4 carbon atoms in the alkyl moiety, aminocarbonyl, alkylaminocarbonyl having 1-4 carbon atoms in the alkyl moiety or dialkylaminocarbonyl having 1-4 carbon atoms each in the alkyl moiety. W³ is preferably hydrogen, cyano, alkoxycarbonyl, aminocarbonyl, alkylaminocarbonyl or dialkylaminocarbonyl, especially preferably cyano.

In a further embodiment of the compounds I, W³ is CR¹⁰R¹¹R¹².

In a further embodiment of the compounds I, W³ is C(R¹³)═NR¹⁴.

Further embodiments of the compounds I correspond to the formulae:

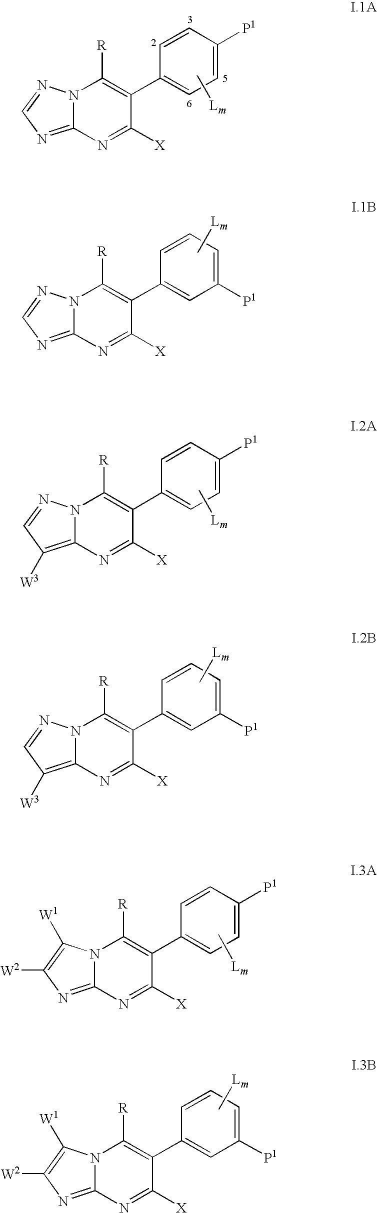

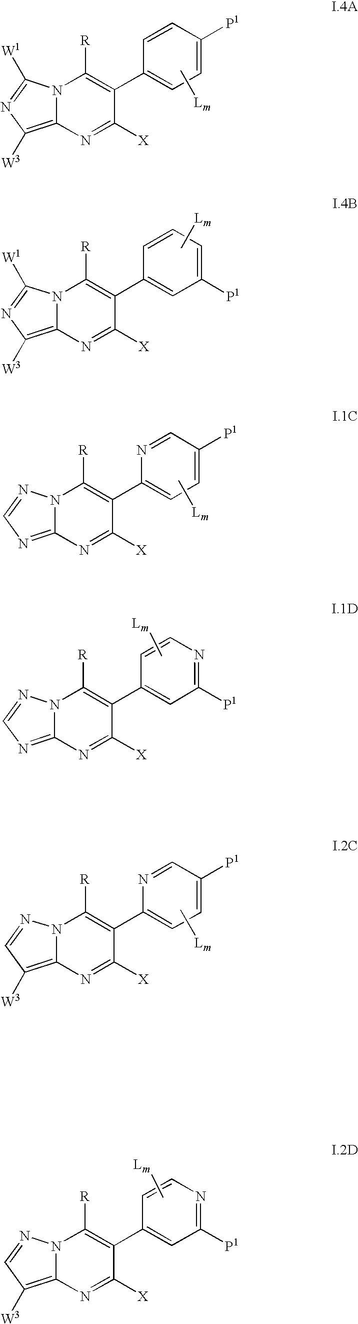

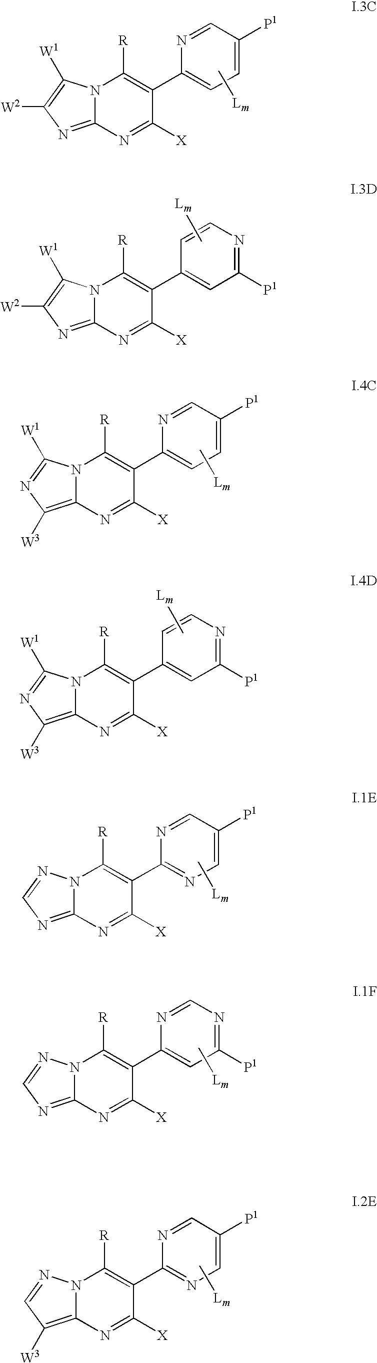

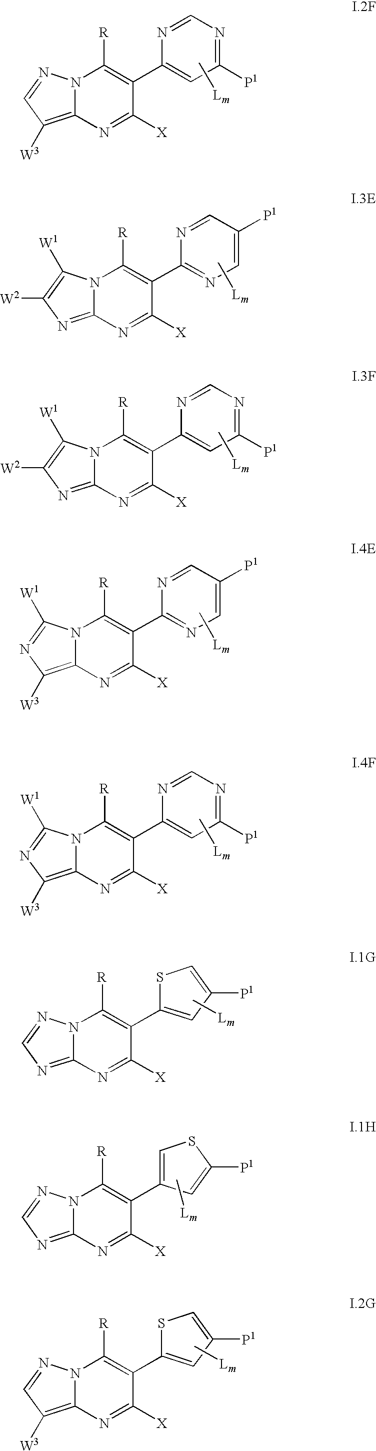

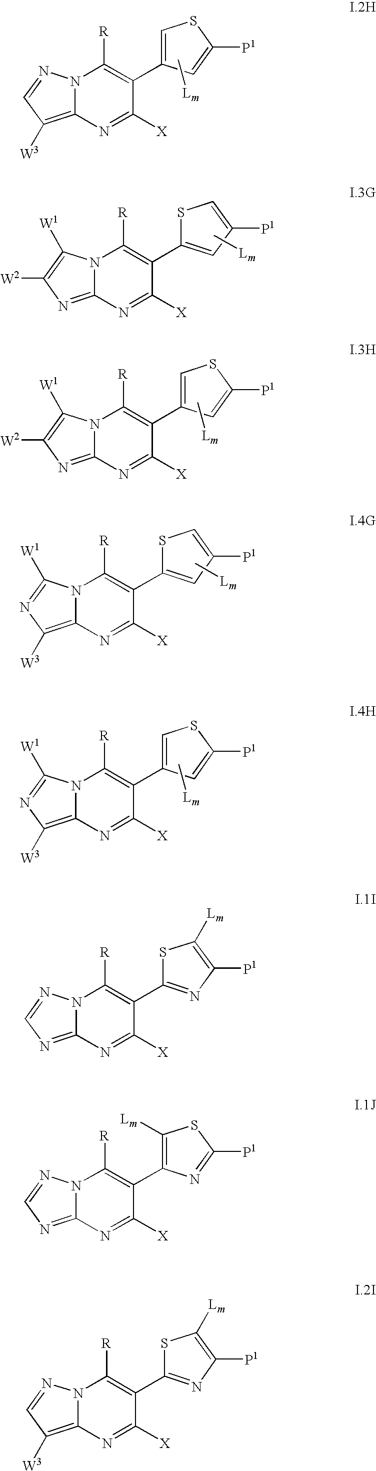

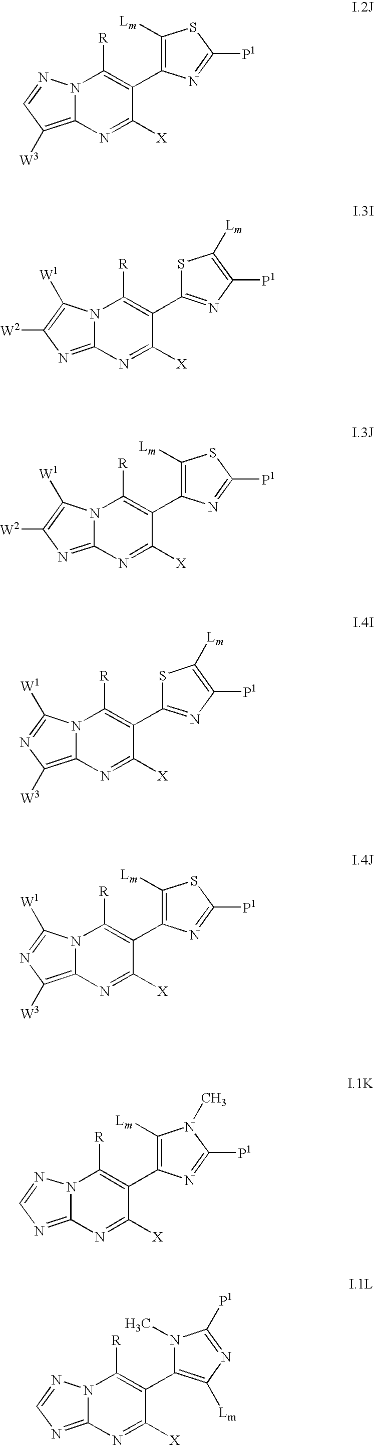

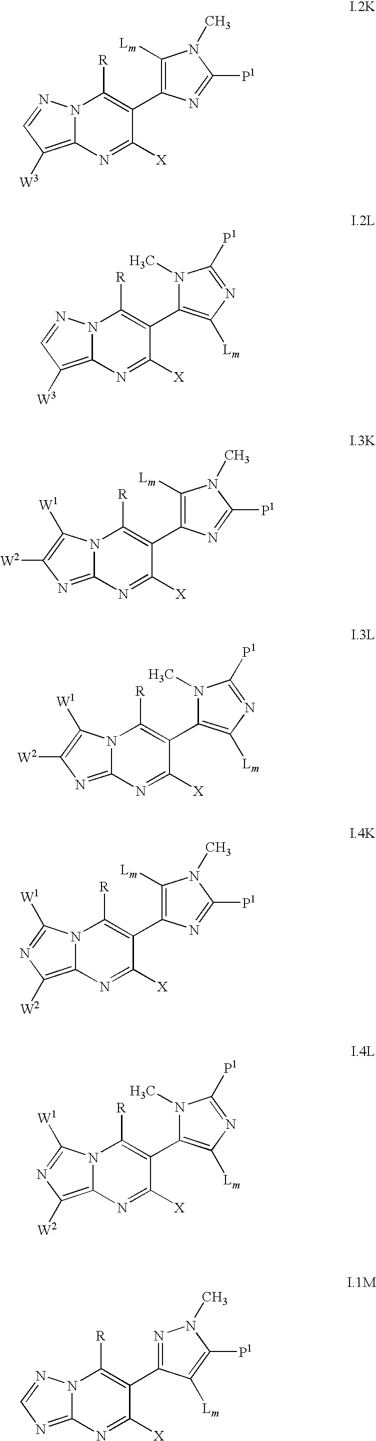

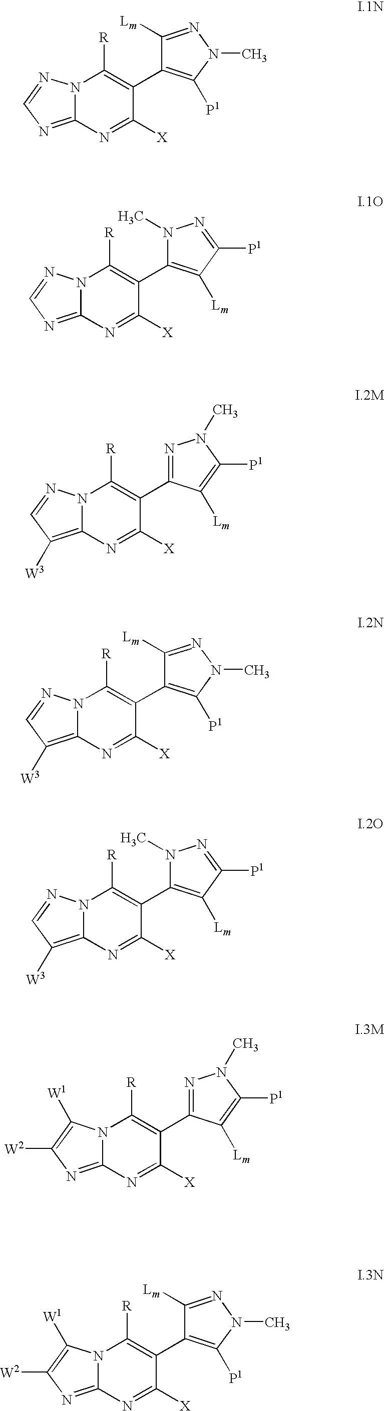

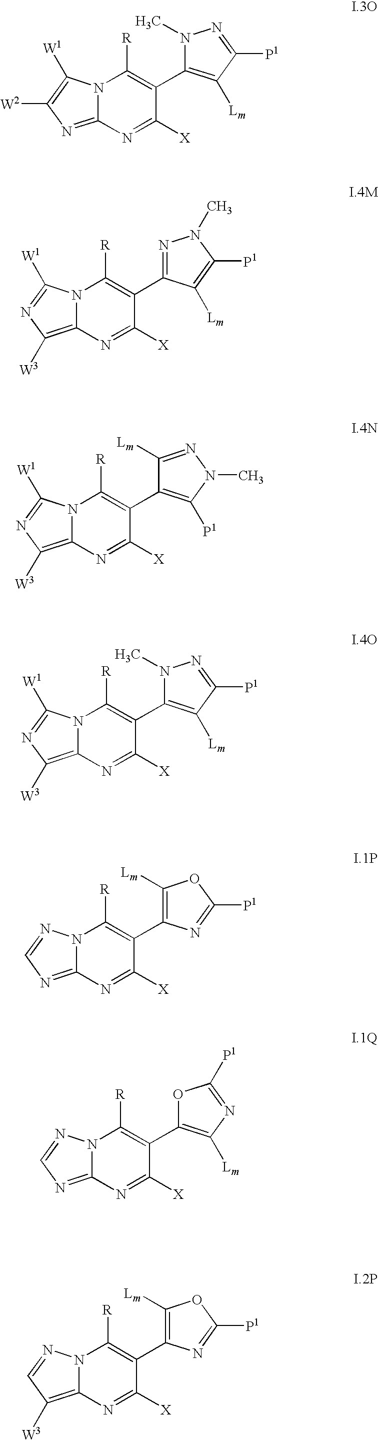

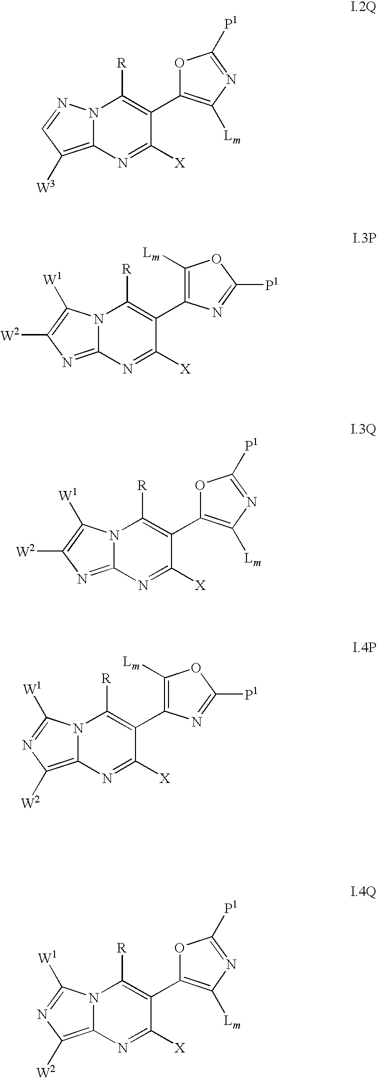

in which the embodiments of the variables correspond to the formula I.

Especially with regard to their use, preference is given to the compounds I compiled in the tables below. The groups specified in the tables for one substituent alone constitute a particularly preferred embodiment of the substituent in question irrespective of the combination in which they are specified.

Tables 1 to 1254 - Compounds of the formula I.1A in which X is Cl, Lₘ and
P¹ are each defined as identified and R is a compound which corresponds
in each case to one line of Table A

| Table | Lₘ | P¹ |
|---|---|---|
| 1 | 2-F | C(O)NH—(CH₂)₂—OH |
| 2 | 2-F | C(O)NH—(CH₂)₂—NH₂ |
| 3 | 2-F | C(O)NH—(CH₂)₂—NHCH₃ |
| 4 | 2-F | C(O)NH—(CH₂)₂—N(CH₃)₂ |
| 5 | 2-F | C(O)NH—(CH₂)₂—O—C(O)H |
| 6 | 2-F | C(O)NH—(CH₂)₃—OH |
| 7 | 2-F | C(O)NH—(CH₂)₃—NH₂ |
| 8 | 2-F | C(O)NH—(CH₂)₃—NHCH₃ |
| 9 | 2-F | C(O)NH—(CH₂)₃—N(CH₃)₂ |
| 10 | 2-F | C(O)NH—(CH₂)₃—O—C(O)H |
| 11 | 2-F | C(O)NH—CH(CH₃)—CH₂—OH |
| 12 | 2-F | C(O)NH—CH(CH₃)—CH₂—NH₂ |
| 13 | 2-F | C(O)NH—CH(CH₃)—CH₂—NHCH₃ |
| 14 | 2-F | C(O)NH—CH(CH₃)—CH₂—N(CH₃)₂ |
| 15 | 2-F | C(O)NH—CH(CH₃)—CH₂—O—C(O)H |
| 16 | 2-F | C(O)NH—CH₂—CH(CH₃)—OH |
| 17 | 2-F | C(O)NH—CH₂—CH(CH₃)—NH₂ |
| 18 | 2-F | C(O)NH—CH₂—CH(CH₃)—NHCH₃ |
| 19 | 2-F | C(O)NH—CH₂—CH(CH₃)—N(CH₃)₂ |
| 20 | 2-F | C(O)NH—CH₂—CH(CH₃)—O—C(O)H |
| 21 | 2-F | C(O)NH—(CH₂)₂—O—(CH₂)₂—OH |
| 22 | 2-F | C(O)NH—(CH₂)₂—O—(CH₂)₂—NH₂ |
| 23 | 2-F | C(O)NH—(CH₂)₂—O—(CH₂)₂—NHCH₃ |
| 24 | 2-F | C(O)NH—(CH₂)₂—O—(CH₂)₂—N(CH₃)₂ |
| 25 | 2-F | C(O)NH—(CH₂)₂—O—(CH₂)₂—O—C(O)H |
| 26 | 2-F | C(O)NH—(CH₂)₂—NH—(CH₂)₂—OH |
| 27 | 2-F | C(O)NH—(CH₂)₂—NH—(CH₂)₂—NH₂ |
| 28 | 2-F | C(O)NH—(CH₂)₂—NH—(CH₂)₂—NHCH₃ |
| 29 | 2-F | C(O)NH—(CH₂)₂—NH—(CH₂)₂—N(CH₃)₂ |
| 30 | 2-F | C(O)NH—(CH₂)₂—NH—(CH₂)₂—O—C(O)H |
| 31 | 2-F | C(O)NH—(CH₂)₂—N(CH₃)—(CH₂)₂—OH |
| 32 | 2-F | C(O)NH—(CH₂)₂—N(CH₃)—(CH₂)₂—NH₂ |
| 33 | 2-F | C(O)NH—(CH₂)₂—N(CH₃)—(CH₂)₂—NHCH₃ |
| 34 | 2-F | C(O)NH—(CH₂)₂—N(CH₃)—(CH₂)₂—N(CH₃)₂ |
| 35 | 2-F | C(O)NH—(CH₂)₂—N(CH₃)—(CH₂)₂—O—C(O)H |
| 36 | 2-F | C(O)N(CH₃)—(CH₂)₂—OH |
| 37 | 2-F | C(O)N(CH₃)—(CH₂)₂—NH₂ |
| 38 | 2-F | C(O)N(CH₃)—(CH₂)₂—NHCH₃ |
| 39 | 2-F | C(O)N(CH₃)—(CH₂)₂—N(CH₃)₂ |
| 40 | 2-F | C(O)N(CH₃)—(CH₂)₂—O—C(O)H |
| 41 | 2-F | C(O)N(CH₃)—(CH₂)₃—OH |
| 42 | 2-F | C(O)N(CH₃)—(CH₂)₃—NH₂ |
| 43 | 2-F | C(O)N(CH₃)—(CH₂)₃—NHCH₃ |
| 44 | 2-F | C(O)N(CH₃)—(CH₂)₃—N(CH₃)₂ |
| 45 | 2-F | C(O)N(CH₃)—(CH₂)₃—O—C(O)H |
| 46 | 2-F | C(O)N(CH₃)—CH(CH₃)—CH₂—OH |
| 47 | 2-F | C(O)N(CH₃)—CH(CH₃)—CH₂—NH₂ |
| 48 | 2-F | C(O)N(CH₃)—CH(CH₃)—CH₂—NHCH₃ |
| 49 | 2-F | C(O)N(CH₃)—CH(CH₃)—CH₂—N(CH₃)₂ |
| 50 | 2-F | C(O)N(CH₃)—CH(CH₃)—CH₂—O—C(O)H |
| 51 | 2-F | C(O)N(CH₃)—CH₂—CH(CH₃)—OH |
| 52 | 2-F | C(O)N(CH₃)—CH₂—CH(CH₃)—NH₂ |
| 53 | 2-F | C(O)N(CH₃)—CH₂—CH(CH₃)—NHCH₃ |
| 54 | 2-F | C(O)N(CH₃)—CH₂—CH(CH₃)—N(CH₃)₂ |
| 55 | 2-F | C(O)N(CH₃)—(CH₂)₂—O—(CH₂)₂—OH |
| 56 | 2-F | C(O)N(CH₃)—(CH₂)₂—O—(CH₂)₂—NH₂ |
| 57 | 2-F | C(O)N(CH₃)—(CH₂)₂—O—(CH₂)₂—NHCH₃ |
| 58 | 2-F | C(O)N(CH₃)—(CH₂)₂—O—(CH₂)₂—N(CH₃)₂ |
| 59 | 2-F | C(O)N(CH₃)—(CH₂)₂—O—(CH₂)₂—O—C(O)H |
| 60 | 2-F | C(O)N(CH₃)—(CH₂)₂—NH—(CH₂)₂—OH |
| 61 | 2-F | C(O)N(CH₃)—(CH₂)₂—NH—(CH₂)₂—NH₂ |
| 62 | 2-F | C(O)N(CH₃)—(CH₂)₂—NH—(CH₂)₂—NHCH₃ |
| 63 | 2-F | C(O)N(CH₃)—(CH₂)₂—NH—(CH₂)₂—N(CH₃)₂ |
| 64 | 2-F | C(O)N(CH₃)—(CH₂)₂—NH—(CH₂)₂—O—C(O)H |
| 65 | 2-F | C(O)N(CH₃)—(CH₂)₂—N(CH₃)—(CH₂)₂—OH |
| 66 | 2-F | C(O)N(CH₃)—(CH₂)₂—N(CH₃)—(CH₂)₂—NH₂ |
| 67 | 2-F | C(O)N(CH₃)—(CH₂)₂—N(CH₃)—(CH₂)₂—NHCH₃ |
| 68 | 2-F | C(O)N(CH₃)—(CH₂)₂—N(CH₃)—(CH₂)₂—N(CH₃)₂ |
| 69 | 2-F | C(O)N(CH₃)—(CH₂)₂—N(CH₃)—(CH₂)₂—O—C(O)H |
| 70 | 2-F | C(O)O—(CH₂)₂—OH |
| 71 | 2-F | C(O)O—(CH₂)₂—NH₂ |
| 72 | 2-F | C(O)O—(CH₂)₂—NHCH₃ |
| 73 | 2-F | C(O)O—(CH₂)₂—N(CH₃)₂ |
| 74 | 2-F | C(O)O—(CH₂)₂—O—C(O)H |
| 75 | 2-F | C(O)O—(CH₂)₃—OH |
| 76 | 2-F | C(O)O—(CH₂)₃—NH₂ |
| 77 | 2-F | C(O)O—(CH₂)₃—NHCH₃ |
| 78 | 2-F | C(O)O—(CH₂)₃—N(CH₃)₂ |
| 79 | 2-F | C(O)O—(CH₂)₃—O—C(O)H |
| 80 | 2-F | C(O)O—CH(CH₃)—CH₂—OH |
| 81 | 2-F | C(O)O—CH(CH₃)—CH₂—NH₂ |
| 82 | 2-F | C(O)O—CH(CH₃)—CH₂—NHCH₃ |
| 83 | 2-F | C(O)O—CH(CH₃)—CH₂—N(CH₃)₂ |
| 84 | 2-F | C(O)O—CH(CH₃)—CH₂—O—C(O)H |
| 85 | 2-F | C(O)O—CH₂—CH(CH₃)—OH |
| 86 | 2-F | C(O)O—CH₂—CH(CH₃)—NH₂ |
| 87 | 2-F | C(O)O—CH₂—CH(CH₃)—NHCH₃ |
| 88 | 2-F | C(O)O—CH₂—CH(CH₃)—N(CH₃)₂ |
| 89 | 2-F | C(O)O—CH₂—CH(CH₃)—O—C(O)H |
| 90 | 2-F | C(O)O—(CH₂)₂—O—(CH₂)₂—OH |
| 91 | 2-F | C(O)O—(CH₂)₂—O—(CH₂)₂—NH₂ |
| 92 | 2-F | C(O)O—(CH₂)₂—O—(CH₂)₂—NHCH₃ |
| 93 | 2-F | C(O)O—(CH₂)₂—O—(CH₂)₂—N(CH₃)₂ |
| 94 | 2-F | C(O)O—(CH₂)₂—O—(CH₂)₂—O—C(O)H |
| 95 | 2-F | C(O)O—(CH₂)₂—NH—(CH₂)₂—OH |
| 96 | 2-F | C(O)O—(CH₂)₂—NH—(CH₂)₂—NH₂ |
| 97 | 2-F | C(O)O—(CH₂)₂—NH—(CH₂)₂—NHCH₃ |
| 98 | 2-F | C(O)O—(CH₂)₂—NH—(CH₂)₂—N(CH₃)₂ |
| 99 | 2-F | C(O)O—(CH₂)₂—NH—(CH₂)₂—O—C(O)H |
| 100 | 2-F | C(O)O—(CH₂)₂—N(CH₃)—(CH₂)₂—OH |
| 101 | 2-F | C(O)O—(CH₂)₂—N(CH₃)—(CH₂)₂—NH₂ |
| 102 | 2-F | C(O)O—(CH₂)₂—N(CH₃)—(CH₂)₂—NHCH₃ |
| 103 | 2-F | C(O)O—(CH₂)₂—N(CH₃)—(CH₂)₂—N(CH₃)₂ |
| 104 | 2-F | C(O)O—(CH₂)₂—N(CH₃)—(CH₂)₂—O—C(O)H |
| 105 | 2-F | O—(CH₂)₃—OCH₃ |
| 106 | 2-F | O—(CH₂)₃-Piperidin-1-yl |
| 107 | 2-F | O—(CH₂)₃-Piperazin-1-yl |
| 108 | 2-F | O—(CH₂)₂-4-Methylpiperazinyl |
| 109 | 2-F | Morpholin-1-yl |
| 110 | 2-F | O—(CH₂)₃—OH |
| 111 | 2-F | O—(CH₂)₃—NH₂ |
| 112 | 2-F | O—(CH₂)₃—NHCH₃ |
| 113 | 2-F | O—(CH₂)₃—N(CH₃)₂ |
| 114 | 2-F | O—(CH₂)₃—O—C(O)H |
| 115 | 2-F | O—CH(CH₃)—CH₂—OH |
| 116 | 2-F | O—CH(CH₃)—CH₂—NH₂ |
| 117 | 2-F | O—CH(CH₃)—CH₂—NHCH₃ |
| 118 | 2-F | O—CH(CH₃)—CH₂—N(CH₃)₂ |
| 119 | 2-F | O—CH(CH₃)—CH₂—O—C(O)H |
| 120 | 2-F | O—CH₂—CH(CH₃)—OH |
| 121 | 2-F | O—CH₂—CH(CH₃)—NH₂ |
| 122 | 2-F | O—CH₂—CH(CH₃)—NHCH₃ |
| 123 | 2-F | O—CH₂—CH(CH₃)—N(CH₃)₂ |
| 124 | 2-F | O—CH₂—CH(CH₃)—O—C(O)H |
| 125 | 2-F | O—(CH₂)₂—O—(CH₂)₂—OH |
| 126 | 2-F | O—(CH₂)₂—O—(CH₂)₂—NH₂ |
| 127 | 2-F | O—(CH₂)₂—O—(CH₂)₂—NHCH₃ |
| 128 | 2-F | O—(CH₂)₂—O—(CH₂)₂—N(CH₃)₂ |
| 129 | 2-F | O—(CH₂)₂—O—(CH₂)₂—O—C(O)H |
| 130 | 2-F | O—(CH₂)₂—NH—(CH₂)₂—OH |
| 131 | 2-F | O—(CH₂)₂—NH—(CH₂)₂—NH₂ |
| 132 | 2-F | O—(CH₂)₂—NH—(CH₂)₂—NHCH₃ |
| 133 | 2-F | O—(CH₂)₂—NH—(CH₂)₂—N(CH₃)₂ |
| 134 | 2-F | O—(CH₂)₂—NH—(CH₂)₂—O—C(O)H |
| 135 | 2-F | O—(CH₂)₂—N(CH₃)—(CH₂)₂—OH |
| 136 | 2-F | O—(CH₂)₂—N(CH₃)—(CH₂)₂—NH₂ |
| 137 | 2-F | O—(CH₂)₂—N(CH₃)—(CH₂)₂—NHCH₃ |
| 138 | 2-F | O—(CH₂)₂—N(CH₃)—(CH₂)₂—N(CH₃)₂ |
| 139 | 2-F | O—(CH₂)₂—N(CH₃)—(CH₂)₂—O—C(O)H |
| 140 | 2-F | NH—(CH₂)₂—OH |
| 141 | 2-F | NH—(CH₂)₂—NH₂ |
| 142 | 2-F | NH—(CH₂)₂—NHCH₃ |
| 143 | 2-F | NH—(CH₂)₂—N(CH₃)₂ |
| 144 | 2-F | NH—(CH₂)₂—O—C(O)H |
| 145 | 2-F | NH—(CH₂)₃—OH |
| 146 | 2-F | NH—(CH₂)₃—NH₂ |
| 147 | 2-F | NH—(CH₂)₃—NHCH₃ |
| 148 | 2-F | NH—(CH₂)₃—N(CH₃)₂ |
| 149 | 2-F | NH—(CH₂)₃—O—C(O)H |
| 150 | 2-F | NH—CH(CH₃)—CH₂—OH |
| 151 | 2-F | NH—CH(CH₃)—CH₂—NH₂ |
| 152 | 2-F | NH—CH(CH₃)—CH₂—NHCH₃ |
| 153 | 2-F | NH—CH(CH₃)—CH₂—N(CH₃)₂ |
| 154 | 2-F | NH—CH(CH₃)—CH₂—O—C(O)H |
| 155 | 2-F | NH—CH₂—CH(CH₃)—OH |
| 156 | 2-F | NH—CH₂—CH(CH₃)—NH₂ |
| 157 | 2-F | NH—CH₂—CH(CH₃)—NHCH₃ |
| 158 | 2-F | NH—CH₂—CH(CH₃)—N(CH₃)₂ |
| 159 | 2-F | NH—CH₂—CH(CH₃)—O—C(O)H |
| 160 | 2-F | NH—(CH₂)₂—O—(CH₂)₂—OH |
| 161 | 2-F | NH—(CH₂)₂—O—(CH₂)₂—NH₂ |
| 162 | 2-F | NH—(CH₂)₂—O—(CH₂)₂—NHCH₃ |
| 163 | 2-F | NH—(CH₂)₂—O—(CH₂)₂—N(CH₃)₂ |
| 164 | 2-F | NH—(CH₂)₂—O—(CH₂)₂—O—C(O)H |
| 165 | 2-F | NH—(CH₂)₂—NH—(CH₂)₂—OH |
| 166 | 2-F | NH—(CH₂)₂—NH—(CH₂)₂—NH₂ |
| 167 | 2-F | NH—(CH₂)₂—NH—(CH₂)₂—NHCH₃ |
| 168 | 2-F | NH—(CH₂)₂—NH—(CH₂)₂—N(CH₃)₂ |
| 169 | 2-F | NH—(CH₂)₂—NH—(CH₂)₂—O—C(O)H |
| 170 | 2-F | NH—(CH₂)₂—N(CH₃)—(CH₂)₂—OH |
| 171 | 2-F | NH—(CH₂)₂—N(CH₃)—(CH₂)₂—NH₂ |
| 172 | 2-F | NH—(CH₂)₂—N(CH₃)—(CH₂)₂—NHCH₃ |
| 173 | 2-F | NH—(CH₂)₂—N(CH₃)—(CH₂)₂—N(CH₃)₂ |
| 174 | 2-F | NH—(CH₂)₂—N(CH₃)—(CH₂)₂—O—C(O)H |
| 175 | 2-F | N(CH₃)—(CH₂)₂—OH |
| 176 | 2-F | N(CH₃)—(CH₂)₂—NH₂ |
| 177 | 2-F | N(CH₃)—(CH₂)₂—NHCH₃ |
| 178 | 2-F | N(CH₃)—(CH₂)₂—N(CH₃)₂ |
| 179 | 2-F | N(CH₃)—(CH₂)₂—O—C(O)H |
| 180 | 2-F | N(CH₃)—(CH₂)₃—OH |
| 181 | 2-F | N(CH₃)—(CH₂)₃—NH₂ |
| 182 | 2-F | N(CH₃)—(CH₂)₃—NHCH₃ |
| 183 | 2-F | N(CH₃)—(CH₂)₃—N(CH₃)₂ |
| 184 | 2-F | N(CH₃)—(CH₂)₃—O—C(O)H |
| 185 | 2-F | N(CH₃)—CH(CH₃)—CH₂—OH |
| 186 | 2-F | N(CH₃)—CH(CH₃)—CH₂—NH₂ |
| 187 | 2-F | N(CH₃)—CH(CH₃)—CH₂—NHCH₃ |
| 188 | 2-F | N(CH₃)—CH(CH₃)—CH₂—N(CH₃)₂ |
| 189 | 2-F | N(CH₃)—CH(CH₃)—CH₂—O—C(O)H |
| 190 | 2-F | N(CH₃)—CH₂—CH(CH₃)—OH |
| 191 | 2-F | N(CH₃)—CH₂—CH(CH₃)—NH₂ |
| 192 | 2-F | N(CH₃)—CH₂—CH(CH₃)—NHCH₃ |
| 193 | 2-F | N(CH₃)—CH₂—CH(CH₃)—N(CH₃)₂ |
| 194 | 2-F | N(CH₃)—CH₂—CH(CH₃)—O—C(O)H |
| 195 | 2-F | N(CH₃)—(CH₂)₂—O—(CH₂)₂—OH |
| 196 | 2-F | N(CH₃)—(CH₂)₂—O—(CH₂)₂—NH₂ |
| 197 | 2-F | N(CH₃)—(CH₂)₂—O—(CH₂)₂—NHCH₃ |
| 198 | 2-F | N(CH₃)—(CH₂)₂—O—(CH₂)₂—N(CH₃)₂ |
| 199 | 2-F | N(CH₃)—(CH₂)₂—O—(CH₂)₂—O—C(O)H |
| 200 | 2-F | N(CH₃)—(CH₂)₂—NH—(CH₂)₂—OH |
| 201 | 2-F | N(CH₃)—(CH₂)₂—NH—(CH₂)₂—NH₂ |
| 202 | 2-F | N(CH₃)—(CH₂)₂—NH—(CH₂)₂—NHCH₃ |
| 203 | 2-F | N(CH₃)—(CH₂)₂—NH—(CH₂)₂—N(CH₃)₂ |
| 204 | 2-F | N(CH₃)—(CH₂)₂—NH—(CH₂)₂—O—C(O)H |
| 205 | 2-F | N(CH₃)—(CH₂)₂—N(CH₃)—(CH₂)₂—OH |
| 206 | 2-F | N(CH₃)—(CH₂)₂—N(CH₃)—(CH₂)₂—NH₂ |
| 207 | 2-F | N(CH₃)—(CH₂)₂—N(CH₃)—(CH₂)₂—NHCH₃ |
| 208 | 2-F | N(CH₃)—(CH₂)₂—N(CH₃)—(CH₂)₂—N(CH₃)₂ |
| 209 | 2-F | N(CH₃)—(CH₂)₂—N(CH₃)—(CH₂)₂—O—C(O)H |
| 210 | 2-Cl | C(O)NH—(CH₂)₂—OH |
| 211 | 2-Cl | C(O)NH—(CH₂)₂—NH₂ |
| 212 | 2-Cl | C(O)NH—(CH₂)₂—NHCH₃ |
| 213 | 2-Cl | C(O)NH—(CH₂)₂—N(CH₃)₂ |
| 214 | 2-Cl | C(O)NH—(CH₂)₂—O—C(O)H |
| 215 | 2-Cl | C(O)NH—(CH₂)₃—OH |
| 216 | 2-Cl | C(O)NH—(CH₂)₃—NH₂ |
| 217 | 2-Cl | C(O)NH—(CH₂)₃—NHCH₃ |
| 218 | 2-Cl | C(O)NH—(CH₂)₃—N(CH₃)₂ |
| 219 | 2-Cl | C(O)NH—(CH₂)₃—O—C(O)H |
| 220 | 2-Cl | C(O)NH—CH(CH₃)—CH₂—OH |
| 221 | 2-Cl | C(O)NH—CH(CH₃)—CH₂—NH₂ |
| 222 | 2-Cl | C(O)NH—CH(CH₃)—CH₂—NHCH₃ |
| 223 | 2-Cl | C(O)NH—CH(CH₃)—CH₂—N(CH₃)₂ |
| 224 | 2-Cl | C(O)NH—CH(CH₃)—CH₂—O—C(O)H |
| 225 | 2-Cl | C(O)NH—CH₂—CH(CH₃)—OH |
| 226 | 2-Cl | C(O)NH—CH₂—CH(CH₃)—NH₂ |
| 227 | 2-Cl | C(O)NH—CH₂—CH(CH₃)—NHCH₃ |
| 228 | 2-Cl | C(O)NH—CH₂—CH(CH₃)—N(CH₃)₂ |
| 229 | 2-Cl | C(O)NH—CH₂—CH(CH₃)—O—C(O)H |
| 230 | 2-Cl | C(O)NH—(CH₂)₂—O—(CH₂)₂—OH |
| 231 | 2-Cl | C(O)NH—(CH₂)₂—O—(CH₂)₂—NH₂ |
| 232 | 2-Cl | C(O)NH—(CH₂)₂—O—(CH₂)₂—NHCH₃ |
| 233 | 2-Cl | C(O)NH—(CH₂)₂—O—(CH₂)₂—N(CH₃)₂ |
| 234 | 2-Cl | C(O)NH—(CH₂)₂—O—(CH₂)₂—O—C(O)H |
| 235 | 2-Cl | C(O)NH—(CH₂)₂—NH—(CH₂)₂—OH |
| 236 | 2-Cl | C(O)NH—(CH₂)₂—NH—(CH₂)₂—NH₂ |
| 237 | 2-Cl | C(O)NH—(CH₂)₂—NH—(CH₂)₂—NHCH₃ |
| 238 | 2-Cl | C(O)NH—(CH₂)₂—NH—(CH₂)₂—N(CH₃)₂ |
| 239 | 2-Cl | C(O)NH—(CH₂)₂—NH—(CH₂)₂—O—C(O)H |
| 240 | 2-Cl | C(O)NH—(CH₂)₂—N(CH₃)—(CH₂)₂—OH |
| 241 | 2-Cl | C(O)NH—(CH₂)₂—N(CH₃)—(CH₂)₂—NH₂ |
| 242 | 2-Cl | C(O)NH—(CH₂)₂—N(CH₃)—(CH₂)₂—NHCH₃ |
| 243 | 2-Cl | C(O)NH—(CH₂)₂—N(CH₃)—(CH₂)₂—N(CH₃)₂ |
| 244 | 2-Cl | C(O)NH—(CH₂)₂—N(CH₃)—(CH₂)₂—O—C(O)H |
| 245 | 2-Cl | C(O)N(CH₃)—(CH₂)₂—OH |
| 246 | 2-Cl | C(O)N(CH₃)—(CH₂)₂—NH₂ |
| 247 | 2-Cl | C(O)N(CH₃)—(CH₂)₂—NHCH₃ |
| 248 | 2-Cl | C(O)N(CH₃)—(CH₂)₂—N(CH₃)₂ |
| 249 | 2-Cl | C(O)N(CH₃)—(CH₂)₂—O—C(O)H |
| 250 | 2-Cl | C(O)N(CH₃)—(CH₂)₃—OH |
| 251 | 2-Cl | C(O)N(CH₃)—(CH₂)₃—NH₂ |
| 252 | 2-Cl | C(O)N(CH₃)—(CH₂)₃—NHCH₃ |
| 253 | 2-Cl | C(O)N(CH₃)—(CH₂)₃—N(CH₃)₂ |
| 254 | 2-Cl | C(O)N(CH₃)—(CH₂)₃—O—C(O)H |
| 255 | 2-Cl | C(O)N(CH₃)—CH(CH₃)—CH₂—OH |
| 256 | 2-Cl | C(O)N(CH₃)—CH(CH₃)—CH₂—NH₂ |
| 257 | 2-Cl | C(O)N(CH₃)—CH(CH₃)—CH₂—NHCH₃ |
| 258 | 2-Cl | C(O)N(CH₃)—CH(CH₃)—CH₂—N(CH₃)₂ |
| 259 | 2-Cl | C(O)N(CH₃)—CH(CH₃)—CH₂—O—C(O)H |
| 260 | 2-Cl | C(O)N(CH₃)—CH₂—CH(CH₃)—OH |
| 261 | 2-Cl | C(O)N(CH₃)—CH₂—CH(CH₃)—NH₂ |
| 262 | 2-Cl | C(O)N(CH₃)—CH₂—CH(CH₃)—NHCH₃ |
| 263 | 2-Cl | C(O)N(CH₃)—CH₂—CH(CH₃)—N(CH₃)₂ |
| 264 | 2-Cl | C(O)N(CH₃)—(CH₂)₂—O—(CH₂)₂—OH |
| 265 | 2-Cl | C(O)N(CH₃)—(CH₂)₂—O—(CH₂)₂—NH₂ |
| 266 | 2-Cl | C(O)N(CH₃)—(CH₂)₂—O—(CH₂)₂—NHCH₃ |
| 267 | 2-Cl | C(O)N(CH₃)—(CH₂)₂—O—(CH₂)₂—N(CH₃)₂ |
| 268 | 2-Cl | C(O)N(CH₃)—(CH₂)₂—O—(CH₂)₂—O—C(O)H |
| 269 | 2-Cl | C(O)N(CH₃)—(CH₂)₂—NH—(CH₂)₂—OH |
| 270 | 2-Cl | C(O)N(CH₃)—(CH₂)₂—NH—(CH₂)₂—NH₂ |
| 271 | 2-Cl | C(O)N(CH₃)—(CH₂)₂—NH—(CH₂)₂—NHCH₃ |
| 272 | 2-Cl | C(O)N(CH₃)—(CH₂)₂—NH—(CH₂)₂—N(CH₃)₂ |
| 273 | 2-Cl | C(O)N(CH₃)—(CH₂)₂—NH—(CH₂)₂—O—C(O)H |
| 274 | 2-Cl | C(O)N(CH₃)—(CH₂)₂—N(CH₃)—(CH₂)₂—OH |
| 275 | 2-Cl | C(O)N(CH₃)—(CH₂)₂—N(CH₃)—(CH₂)₂—NH₂ |
| 276 | 2-Cl | C(O)N(CH₃)—(CH₂)₂—N(CH₃)—(CH₂)₂—NHCH₃ |
| 277 | 2-Cl | C(O)N(CH₃)—(CH₂)₂—N(CH₃)—(CH₂)₂—N(CH₃)₂ |
| 278 | 2-Cl | C(O)N(CH₃)—(CH₂)₂—N(CH₃)—(CH₂)₂—O—C(O)H |
| 279 | 2-Cl | C(O)O—(CH₂)₂—OH |
| 280 | 2-Cl | C(O)O—(CH₂)₂—NH₂ |
| 281 | 2-Cl | C(O)O—(CH₂)₂—NHCH₃ |
| 282 | 2-Cl | C(O)O—(CH₂)₂—N(CH₃)₂ |
| 283 | 2-Cl | C(O)O—(CH₂)₂—O—C(O)H |
| 284 | 2-Cl | C(O)O—(CH₂)₃—OH |
| 285 | 2-Cl | C(O)O—(CH₂)₃—NH₂ |
| 286 | 2-Cl | C(O)O—(CH₂)₃—NHCH₃ |
| 287 | 2-Cl | C(O)O—(CH₂)₃—N(CH₃)₂ |
| 288 | 2-Cl | C(O)O—(CH₂)₃—O—C(O)H |
| 289 | 2-Cl | C(O)O—CH(CH₃)—CH₂—OH |
| 290 | 2-Cl | C(O)O—CH(CH₃)—CH₂—NH₂ |
| 291 | 2-Cl | C(O)O—CH(CH₃)—CH₂—NHCH₃ |
| 292 | 2-Cl | C(O)O—CH(CH₃)—CH₂—N(CH₃)₂ |
| 293 | 2-Cl | C(O)O—CH(CH₃)—CH₂—O—C(O)H |
| 294 | 2-Cl | C(O)O—CH₂—CH(CH₃)—OH |
| 295 | 2-Cl | C(O)O—CH₂—CH(CH₃)—NH₂ |
| 296 | 2-Cl | C(O)O—CH₂—CH(CH₃)—NHCH₃ |
| 297 | 2-Cl | C(O)O—CH₂—CH(CH₃)—N(CH₃)₂ |
| 298 | 2-Cl | C(O)O—CH₂—CH(CH₃)—O—C(O)H |
| 299 | 2-Cl | C(O)O—(CH₂)₂—O—(CH₂)₂—OH |
| 300 | 2-Cl | C(O)O—(CH₂)₂—O—(CH₂)₂—NH₂ |
| 301 | 2-Cl | C(O)O—(CH₂)₂—O—(CH₂)₂—NHCH₃ |
| 302 | 2-Cl | C(O)O—(CH₂)₂—O—(CH₂)₂—N(CH₃)₂ |
| 303 | 2-Cl | C(O)O—(CH₂)₂—O—(CH₂)₂—O—C(O)H |
| 304 | 2-Cl | C(O)O—(CH₂)₂—NH—(CH₂)₂—OH |
| 305 | 2-Cl | C(O)O—(CH₂)₂—NH—(CH₂)₂—NH₂ |
| 306 | 2-Cl | C(O)O—(CH₂)₂—NH—(CH₂)₂—NHCH₃ |
| 307 | 2-Cl | C(O)O—(CH₂)₂—NH—(CH₂)₂—N(CH₃)₂ |
| 308 | 2-Cl | C(O)O—(CH₂)₂—NH—(CH₂)₂—O—C(O)H |
| 309 | 2-Cl | C(O)O—(CH₂)₂—N(CH₃)—(CH₂)₂—OH |
| 310 | 2-Cl | C(O)O—(CH₂)₂—N(CH₃)—(CH₂)₂—NH₂ |
| 311 | 2-Cl | C(O)O—(CH₂)₂—N(CH₃)—(CH₂)₂—NHCH₃ |
| 312 | 2-Cl | C(O)O—(CH₂)₂—N(CH₃)—(CH₂)₂—N(CH₃)₂ |
| 313 | 2-Cl | C(O)O—(CH₂)₂—N(CH₃)—(CH₂)₂—O—C(O)H |
| 314 | 2-Cl | O—(CH₂)₃—OCH₃ |
| 315 | 2-Cl | O—(CH₂)₃-Piperidin-1-yl |
| 316 | 2-Cl | O—(CH₂)₃-Piperazin-1-yl |
| 317 | 2-Cl | O—(CH₂)₂-4-Methylpiperazinyl |
| 318 | 2-Cl | Morpholin-1-yl |
| 319 | 2-Cl | O—(CH₂)₃—OH |
| 320 | 2-Cl | O—(CH₂)₃—NH₂ |
| 321 | 2-Cl | O—(CH₂)₃—NHCH₃ |
| 322 | 2-Cl | O—(CH₂)₃—N(CH₃)₂ |
| 323 | 2-Cl | O—(CH₂)₃—O—C(O)H |
| 324 | 2-Cl | O—CH(CH₃)—CH₂—OH |
| 325 | 2-Cl | O—CH(CH₃)—CH₂—NH₂ |
| 326 | 2-Cl | O—CH(CH₃)—CH₂—NHCH₃ |
| 327 | 2-Cl | O—CH(CH₃)—CH₂—N(CH₃)₂ |
| 328 | 2-Cl | O—CH(CH₃)—CH₂—O—C(O)H |
| 329 | 2-Cl | O—CH₂—CH(CH₃)—OH |
| 330 | 2-Cl | O—CH₂—CH(CH₃)—NH₂ |
| 331 | 2-Cl | O—CH₂—CH(CH₃)—NHCH₃ |
| 332 | 2-Cl | O—CH₂—CH(CH₃)—N(CH₃)₂ |
| 333 | 2-Cl | O—CH₂—CH(CH₃)—O—C(O)H |
| 334 | 2-Cl | O—(CH₂)₂—O—(CH₂)₂—OH |
| 335 | 2-Cl | O—(CH₂)₂—O—(CH₂)₂—NH₂ |
| 336 | 2-Cl | O—(CH₂)₂—O—(CH₂)₂—NHCH₃ |
| 337 | 2-Cl | O—(CH₂)₂—O—(CH₂)₂—N(CH₃)₂ |
| 338 | 2-Cl | O—(CH₂)₂—O—(CH₂)₂—O—C(O)H |
| 339 | 2-Cl | O—(CH₂)₂—NH—(CH₂)₂—OH |
| 340 | 2-Cl | O—(CH₂)₂—NH—(CH₂)₂—NH₂ |
| 341 | 2-Cl | O—(CH₂)₂—NH—(CH₂)₂—NHCH₃ |
| 342 | 2-Cl | O—(CH₂)₂—NH—(CH₂)₂—N(CH₃)₂ |
| 343 | 2-Cl | O—(CH₂)₂—NH—(CH₂)₂—O—C(O)H |
| 344 | 2-Cl | O—(CH₂)₂—N(CH₃)—(CH₂)₂—OH |
| 345 | 2-Cl | O—(CH₂)₂—N(CH₃)—(CH₂)₂—NH₂ |
| 346 | 2-Cl | O—(CH₂)₂—N(CH₃)—(CH₂)₂—NHCH₃ |
| 347 | 2-Cl | O—(CH₂)₂—N(CH₃)—(CH₂)₂—N(CH₃)₂ |
| 348 | 2-Cl | O—(CH₂)₂—N(CH₃)—(CH₂)₂—O—C(O)H |
| 349 | 2-Cl | NH—(CH₂)₂—OH |
| 350 | 2-Cl | NH—(CH₂)₂—NH₂ |
| 351 | 2-Cl | NH—(CH₂)₂—NHCH₃ |
| 352 | 2-Cl | NH—(CH₂)₂—N(CH₃)₂ |
| 353 | 2-Cl | NH—(CH₂)₂—O—C(O)H |
| 354 | 2-Cl | NH—(CH₂)₃—OH |
| 355 | 2-Cl | NH—(CH₂)₃—NH₂ |
| 356 | 2-Cl | NH—(CH₂)₃—NHCH₃ |
| 357 | 2-Cl | NH—(CH₂)₃—N(CH₃)₂ |
| 358 | 2-Cl | NH—(CH₂)₃—O—C(O)H |
| 359 | 2-Cl | NH—CH(CH₃)—CH₂—OH |
| 360 | 2-Cl | NH—CH(CH₃)—CH₂—NH₂ |
| 361 | 2-Cl | NH—CH(CH₃)—CH₂—NHCH₃ |
| 362 | 2-Cl | NH—CH(CH₃)—CH₂—N(CH₃)₂ |
| 363 | 2-Cl | NH—CH(CH₃)—CH₂—O—C(O)H |
| 364 | 2-Cl | NH—CH₂—CH(CH₃)—OH |
| 365 | 2-Cl | NH—CH₂—CH(CH₃)—NH₂ |
| 366 | 2-Cl | NH—CH₂—CH(CH₃)—NHCH₃ |
| 367 | 2-Cl | NH—CH₂—CH(CH₃)—N(CH₃)₂ |
| 368 | 2-Cl | NH—CH₂—CH(CH₃)—O—C(O)H |
| 369 | 2-Cl | NH—(CH₂)₂—O—(CH₂)₂—OH |
| 370 | 2-Cl | NH—(CH₂)₂—O—(CH₂)₂—NH₂ |
| 371 | 2-Cl | NH—(CH₂)₂—O—(CH₂)₂—NHCH₃ |
| 372 | 2-Cl | NH—(CH₂)₂—O—(CH₂)₂—N(CH₃)₂ |
| 373 | 2-Cl | NH—(CH₂)₂—O—(CH₂)₂—O—C(O)H |
| 374 | 2-Cl | NH—(CH₂)₂—NH—(CH₂)₂—OH |
| 375 | 2-Cl | NH—(CH₂)₂—NH—(CH₂)₂—NH₂ |
| 376 | 2-Cl | NH—(CH₂)₂—NH—(CH₂)₂—NHCH₃ |
| 377 | 2-Cl | NH—(CH₂)₂—NH—(CH₂)₂—N(CH₃)₂ |
| 378 | 2-Cl | NH—(CH₂)₂—NH—(CH₂)₂—O—C(O)H |
| 379 | 2-Cl | NH—(CH₂)₂—N(CH₃)—(CH₂)₂—OH |
| 380 | 2-Cl | NH—(CH₂)₂—N(CH₃)—(CH₂)₂—NH₂ |
| 381 | 2-Cl | NH—(CH₂)₂—N(CH₃)—(CH₂)₂—NHCH₃ |
| 382 | 2-Cl | NH—(CH₂)₂—N(CH₃)—(CH₂)₂—N(CH₃)₂ |
| 383 | 2-Cl | NH—(CH₂)₂—N(CH₃)—(CH₂)₂—O—C(O)H |
| 384 | 2-Cl | N(CH₃)—(CH₂)₂—OH |
| 385 | 2-Cl | N(CH₃)—(CH₂)₂—NH₂ |
| 386 | 2-Cl | N(CH₃)—(CH₂)₂—NHCH₃ |
| 387 | 2-Cl | N(CH₃)—(CH₂)₂—N(CH₃)₂ |
| 388 | 2-Cl | N(CH₃)—(CH₂)₂—O—C(O)H |
| 389 | 2-Cl | N(CH₃)—(CH₂)₃—OH |
| 390 | 2-Cl | N(CH₃)—(CH₂)₃—NH₂ |
| 391 | 2-Cl | N(CH₃)—(CH₂)₃—NHCH₃ |
| 392 | 2-Cl | N(CH₃)—(CH₂)₃—N(CH₃)₂ |
| 393 | 2-Cl | N(CH₃)—(CH₂)₃—O—C(O)H |
| 394 | 2-Cl | N(CH₃)—CH(CH₃)—CH₂—OH |
| 395 | 2-Cl | N(CH₃)—CH(CH₃)—CH₂—NH₂ |
| 396 | 2-Cl | N(CH₃)—CH(CH₃)—CH₂—NHCH₃ |
| 397 | 2-Cl | N(CH₃)—CH(CH₃)—CH₂—N(CH₃)₂ |
| 398 | 2-Cl | N(CH₃)—CH(CH₃)—CH₂—O—C(O)H |
| 399 | 2-Cl | N(CH₃)—CH₂—CH(CH₃)—OH |
| 400 | 2-Cl | N(CH₃)—CH₂—CH(CH₃)—NH₂ |
| 401 | 2-Cl | N(CH₃)—CH₂—CH(CH₃)—NHCH₃ |
| 402 | 2-Cl | N(CH₃)—CH₂—CH(CH₃)—N(CH₃)₂ |
| 403 | 2-Cl | N(CH₃)—CH₂—CH(CH₃)—O—C(O)H |
| 404 | 2-Cl | N(CH₃)—(CH₂)₂—O—(CH₂)₂—OH |
| 405 | 2-Cl | N(CH₃)—(CH₂)₂—O—(CH₂)₂—NH₂ |
| 406 | 2-Cl | N(CH₃)—(CH₂)₂—O—(CH₂)₂—NHCH₃ |
| 407 | 2-Cl | N(CH₃)—(CH₂)₂—O—(CH₂)₂—N(CH₃)₂ |
| 408 | 2-Cl | N(CH₃)—(CH₂)₂—O—(CH₂)₂—O—C(O)H |
| 409 | 2-Cl | N(CH₃)—(CH₂)₂—NH—(CH₂)₂—OH |
| 410 | 2-Cl | N(CH₃)—(CH₂)₂—NH—(CH₂)₂—NH₂ |
| 411 | 2-Cl | N(CH₃)—(CH₂)₂—NH—(CH₂)₂—NHCH₃ |
| 412 | 2-Cl | N(CH₃)—(CH₂)₂—NH—(CH₂)₂—N(CH₃)₂ |
| 413 | 2-Cl | N(CH₃)—(CH₂)₂—NH—(CH₂)₂—O—C(O)H |
| 414 | 2-Cl | N(CH₃)—(CH₂)₂—N(CH₃)—(CH₂)₂—OH |
| 415 | 2-Cl | N(CH₃)—(CH₂)₂—N(CH₃)—(CH₂)₂—NH₂ |
| 416 | 2-Cl | N(CH₃)—(CH₂)₂—N(CH₃)—(CH₂)₂—NHCH₃ |
| 417 | 2-Cl | N(CH₃)—(CH₂)₂—N(CH₃)—(CH₂)₂—N(CH₃)₂ |
| 418 | 2-Cl | N(CH₃)—(CH₂)₂—N(CH₃)—(CH₂)₂—O—C(O)H |
| 419 | 2-CH₃ | C(O)NH—(CH₂)₂—OH |
| 420 | 2-CH₃ | C(O)NH—(CH₂)₂—NH₂ |
| 421 | 2-CH₃ | C(O)NH—(CH₂)₂—NHCH₃ |
| 422 | 2-CH₃ | C(O)NH—(CH₂)₂—N(CH₃)₂ |
| 423 | 2-CH₃ | C(O)NH—(CH₂)₂—O—C(O)H |
| 424 | 2-CH₃ | C(O)NH—(CH₂)₃—OH |
| 425 | 2-CH₃ | C(O)NH—(CH₂)₃—NH₂ |
| 426 | 2-CH₃ | C(O)NH—(CH₂)₃—NHCH₃ |
| 427 | 2-CH₃ | C(O)NH—(CH₂)₃—N(CH₃)₂ |
| 428 | 2-CH₃ | C(O)NH—(CH₂)₃—O—C(O)H |
| 429 | 2-CH₃ | C(O)NH—CH(CH₃)—CH₂—OH |
| 430 | 2-CH₃ | C(O)NH—CH(CH₃)—CH₂—NH₂ |
| 431 | 2-CH₃ | C(O)NH—CH(CH₃)—CH₂—NHCH₃ |
| 432 | 2-CH₃ | C(O)NH—CH(CH₃)—CH₂—N(CH₃)₂ |
| 433 | 2-CH₃ | C(O)NH—CH(CH₃)—CH₂—O—C(O)H |
| 434 | 2-CH₃ | C(O)NH—CH₂—CH(CH₃)—OH |
| 435 | 2-CH₃ | C(O)NH—CH₂—CH(CH₃)—NH₂ |
| 436 | 2-CH₃ | C(O)NH—CH₂—CH(CH₃)—NHCH₃ |
| 437 | 2-CH₃ | C(O)NH—CH₂—CH(CH₃)—N(CH₃)₂ |
| 438 | 2-CH₃ | C(O)NH—CH₂—CH(CH₃)—O—C(O)H |
| 439 | 2-CH₃ | C(O)NH—(CH₂)₂—O—(CH₂)₂—OH |
| 440 | 2-CH₃ | C(O)NH—(CH₂)₂—O—(CH₂)₂—NH₂ |
| 441 | 2-CH₃ | C(O)NH—(CH₂)₂—O—(CH₂)₂—NHCH₃ |
| 442 | 2-CH₃ | C(O)NH—(CH₂)₂—O—(CH₂)₂—N(CH₃)₂ |
| 443 | 2-CH₃ | C(O)NH—(CH₂)₂—O—(CH₂)₂—O—C(O)H |
| 444 | 2-CH₃ | C(O)NH—(CH₂)₂—NH—(CH₂)₂—OH |
| 445 | 2-CH₃ | C(O)NH—(CH₂)₂—NH—(CH₂)₂—NH₂ |
| 446 | 2-CH₃ | C(O)NH—(CH₂)₂—NH—(CH₂)₂—NHCH₃ |
| 447 | 2-CH₃ | C(O)NH—(CH₂)₂—NH—(CH₂)₂—N(CH₃)₂ |
| 448 | 2-CH₃ | C(O)NH—(CH₂)₂—NH—(CH₂)₂—O—C(O)H |
| 449 | 2-CH₃ | C(O)NH—(CH₂)₂—N(CH₃)—(CH₂)₂—OH |
| 450 | 2-CH₃ | C(O)NH—(CH₂)₂—N(CH₃)—(CH₂)₂—NH₂ |
| 451 | 2-CH₃ | C(O)NH—(CH₂)₂—N(CH₃)—(CH₂)₂—NHCH₃ |
| 452 | 2-CH₃ | C(O)NH—(CH₂)₂—N(CH₃)—(CH₂)₂—N(CH₃)₂ |
| 453 | 2-CH₃ | C(O)NH—(CH₂)₂—N(CH₃)—(CH₂)₂—O—C(O)H |
| 454 | 2-CH₃ | C(O)N(CH₃)—(CH₂)₂—OH |
| 455 | 2-CH₃ | C(O)N(CH₃)—(CH₂)₂—NH₂ |
| 456 | 2-CH₃ | C(O)N(CH₃)—(CH₂)₂—NHCH₃ |
| 457 | 2-CH₃ | C(O)N(CH₃)—(CH₂)₂—N(CH₃)₂ |
| 458 | 2-CH₃ | C(O)N(CH₃)—(CH₂)₂—O—C(O)H |
| 459 | 2-CH₃ | C(O)N(CH₃)—(CH₂)₃—OH |
| 460 | 2-CH₃ | C(O)N(CH₃)—(CH₂)₃—NH₂ |
| 461 | 2-CH₃ | C(O)N(CH₃)—(CH₂)₃—NHCH₃ |
| 462 | 2-CH₃ | C(O)N(CH₃)—(CH₂)₃—N(CH₃)₂ |
| 463 | 2-CH₃ | C(O)N(CH₃)—(CH₂)₃—O—C(O)H |
| 464 | 2-CH₃ | C(O)N(CH₃)—CH(CH₃)—CH₂—OH |
| 465 | 2-CH₃ | C(O)N(CH₃)—CH(CH₃)—CH₂—NH₂ |
| 466 | 2-CH₃ | C(O)N(CH₃)—CH(CH₃)—CH₂—NHCH₃ |
| 467 | 2-CH₃ | C(O)N(CH₃)—CH(CH₃)—CH₂—N(CH₃)₂ |
| 468 | 2-CH₃ | C(O)N(CH₃)—CH(CH₃)—CH₂—O—C(O)H |
| 469 | 2-CH₃ | C(O)N(CH₃)—CH₂—CH(CH₃)—OH |
| 470 | 2-CH₃ | C(O)N(CH₃)—CH₂—CH(CH₃)—NH₂ |
| 471 | 2-CH₃ | C(O)N(CH₃)—CH₂—CH(CH₃)—NHCH₃ |
| 472 | 2-CH₃ | C(O)N(CH₃)—CH₂—CH(CH₃)—N(CH₃)₂ |
| 473 | 2-CH₃ | C(O)N(CH₃)—(CH₂)₂—O—(CH₂)₂—OH |
| 474 | 2-CH₃ | C(O)N(CH₃)—(CH₂)₂—O—(CH₂)₂—NH₂ |
| 475 | 2-CH₃ | C(O)N(CH₃)—(CH₂)₂—O—(CH₂)₂—NHCH₃ |
| 476 | 2-CH₃ | C(O)N(CH₃)—(CH₂)₂—O—(CH₂)₂—N(CH₃)₂ |
| 477 | 2-CH₃ | C(O)N(CH₃)—(CH₂)₂—O—(CH₂)₂—O—C(O)H |
| 478 | 2-CH₃ | C(O)N(CH₃)—(CH₂)₂—NH—(CH₂)₂—OH |
| 479 | 2-CH₃ | C(O)N(CH₃)—(CH₂)₂—NH—(CH₂)₂—NH₂ |
| 480 | 2-CH₃ | C(O)N(CH₃)—(CH₂)₂—NH—(CH₂)₂—NHCH₃ |
| 481 | 2-CH₃ | C(O)N(CH₃)—(CH₂)₂—NH—(CH₂)₂—N(CH₃)₂ |
| 482 | 2-CH₃ | C(O)N(CH₃)—(CH₂)₂—NH—(CH₂)₂—O—C(O)H |
| 483 | 2-CH₃ | C(O)N(CH₃)—(CH₂)₂—N(CH₃)—(CH₂)₂—OH |
| 484 | 2-CH₃ | C(O)N(CH₃)—(CH₂)₂—N(CH₃)—(CH₂)₂—NH₂ |
| 485 | 2-CH₃ | C(O)N(CH₃)—(CH₂)₂—N(CH₃)—(CH₂)₂—NHCH₃ |
| 486 | 2-CH₃ | C(O)N(CH₃)—(CH₂)₂—N(CH₃)—(CH₂)₂—N(CH₃)₂ |
| 487 | 2-CH₃ | C(O)N(CH₃)—(CH₂)₂—N(CH₃)—(CH₂)₂—O—C(O)H |
| 488 | 2-CH₃ | C(O)O—(CH₂)₂—OH |
| 489 | 2-CH₃ | C(O)O—(CH₂)₂—NH₂ |
| 490 | 2-CH₃ | C(O)O—(CH₂)₂—NHCH₃ |
| 491 | 2-CH₃ | C(O)O—(CH₂)₂—N(CH₃)₂ |
| 492 | 2-CH₃ | C(O)O—(CH₂)₂—O—C(O)H |
| 493 | 2-CH₃ | C(O)O—(CH₂)₃—OH |
| 494 | 2-CH₃ | C(O)O—(CH₂)₃—NH₂ |
| 495 | 2-CH₃ | C(O)O—(CH₂)₃—NHCH₃ |
| 496 | 2-CH₃ | C(O)O—(CH₂)₃—N(CH₃)₂ |
| 497 | 2-CH₃ | C(O)O—(CH₂)₃—O—C(O)H |
| 498 | 2-CH₃ | C(O)O—CH(CH₃)—CH₂—OH |
| 499 | 2-CH₃ | C(O)O—CH(CH₃)—CH₂—NH₂ |
| 500 | 2-CH₃ | C(O)O—CH(CH₃)—CH₂—NHCH₃ |
| 501 | 2-CH₃ | C(O)O—CH(CH₃)—CH₂—N(CH₃)₂ |
| 502 | 2-CH₃ | C(O)O—CH(CH₃)—CH₂—O—C(O)H |
| 503 | 2-CH₃ | C(O)O—CH₂—CH(CH₃)—OH |
| 504 | 2-CH₃ | C(O)O—CH₂—CH(CH₃)—NH₂ |
| 505 | 2-CH₃ | C(O)O—CH₂—CH(CH₃)—NHCH₃ |
| 506 | 2-CH₃ | C(O)O—CH₂—CH(CH₃)—N(CH₃)₂ |
| 507 | 2-CH₃ | C(O)O—CH₂—CH(CH₃)—O—C(O)H |
| 508 | 2-CH₃ | C(O)O—(CH₂)₂—O—(CH₂)₂—OH |
| 509 | 2-CH₃ | C(O)O—(CH₂)₂—O—(CH₂)₂—NH₂ |
| 510 | 2-CH₃ | C(O)O—(CH₂)₂—O—(CH₂)₂—NHCH₃ |
| 511 | 2-CH₃ | C(O)O—(CH₂)₂—O—(CH₂)₂—N(CH₃)₂ |
| 512 | 2-CH₃ | C(O)O—(CH₂)₂—O—(CH₂)₂—O—C(O)H |
| 513 | 2-CH₃ | C(O)O—(CH₂)₂—NH—(CH₂)₂—OH |
| 514 | 2-CH₃ | C(O)O—(CH₂)₂—NH—(CH₂)₂—NH₂ |
| 515 | 2-CH₃ | C(O)O—(CH₂)₂—NH—(CH₂)₂—NHCH₃ |
| 516 | 2-CH₃ | C(O)O—(CH₂)₂—NH—(CH₂)₂—N(CH₃)₂ |
| 517 | 2-CH₃ | C(O)O—(CH₂)₂—NH—(CH₂)₂—O—C(O)H |
| 518 | 2-CH₃ | C(O)O—(CH₂)₂—N(CH₃)—(CH₂)₂—OH |
| 519 | 2-CH₃ | C(O)O—(CH₂)₂—N(CH₃)—(CH₂)₂—NH₂ |
| 520 | 2-CH₃ | C(O)O—(CH₂)₂—N(CH₃)—(CH₂)₂—NHCH₃ |
| 521 | 2-CH₃ | C(O)O—(CH₂)₂—N(CH₃)—(CH₂)₂—N(CH₃)₂ |
| 522 | 2-CH₃ | C(O)O—(CH₂)₂—N(CH₃)—(CH₂)₂—O—C(O)H |
| 523 | 2-CH₃ | O—(CH₂)₃—OCH₃ |
| 524 | 2-CH₃ | O—(CH₂)₃-Piperidin-1-yl |
| 525 | 2-CH₃ | O—(CH₂)₃-Piperazin-1-yl |
| 526 | 2-CH₃ | O—(CH₂)₂-4-Methylpiperazinyl |
| 527 | 2-CH₃ | Morpholin-1-yl |
| 528 | 2-CH₃ | O—(CH₂)₃—OH |
| 529 | 2-CH₃ | O—(CH₂)₃—NH₂ |
| 530 | 2-CH₃ | O—(CH₂)₃—NHCH₃ |
| 531 | 2-CH₃ | O—(CH₂)₃—N(CH₃)₂ |
| 532 | 2-CH₃ | O—(CH₂)₃—O—C(O)H |
| 533 | 2-CH₃ | O—CH(CH₃)—CH₂—OH |
| 534 | 2-CH₃ | O—CH(CH₃)—CH₂—NH₂ |
| 535 | 2-CH₃ | O—CH(CH₃)—CH₂—NHCH₃ |
| 536 | 2-CH₃ | O—CH(CH₃)—CH₂—N(CH₃)₂ |
| 537 | 2-CH₃ | O—CH(CH₃)—CH₂—O—C(O)H |
| 538 | 2-CH₃ | O—CH₂—CH(CH₃)—OH |
| 539 | 2-CH₃ | O—CH₂—CH(CH₃)—NH₂ |
| 540 | 2-CH₃ | O—CH₂—CH(CH₃)—NHCH₃ |
| 541 | 2-CH₃ | O—CH₂—CH(CH₃)—N(CH₃)₂ |
| 542 | 2-CH₃ | O—CH₂—CH(CH₃)—O—C(O)H |
| 543 | 2-CH₃ | O—(CH₂)₂—O—(CH₂)₂—OH |
| 544 | 2-CH₃ | O—(CH₂)₂—O—(CH₂)₂—NH₂ |
| 545 | 2-CH₃ | O—(CH₂)₂—O—(CH₂)₂—NHCH₃ |
| 546 | 2-CH₃ | O—(CH₂)₂—O—(CH₂)₂—N(CH₃)₂ |
| 547 | 2-CH₃ | O—(CH₂)₂—O—(CH₂)₂—O—C(O)H |
| 548 | 2-CH₃ | O—(CH₂)₂—NH—(CH₂)₂—OH |
| 549 | 2-CH₃ | O—(CH₂)₂—NH—(CH₂)₂—NH₂ |
| 550 | 2-CH₃ | O—(CH₂)₂—NH—(CH₂)₂—NHCH₃ |
| 551 | 2-CH₃ | O—(CH₂)₂—NH—(CH₂)₂—N(CH₃)₂ |
| 552 | 2-CH₃ | O—(CH₂)₂—NH—(CH₂)₂—O—C(O)H |
| 553 | 2-CH₃ | O—(CH₂)₂—N(CH₃)—(CH₂)₂—OH |
| 554 | 2-CH₃ | O—(CH₂)₂—N(CH₃)—(CH₂)₂—NH₂ |
| 555 | 2-CH₃ | O—(CH₂)₂—N(CH₃)—(CH₂)₂—NHCH₃ |
| 556 | 2-CH₃ | O—(CH₂)₂—N(CH₃)—(CH₂)₂—N(CH₃)₂ |
| 557 | 2-CH₃ | O—(CH₂)₂—N(CH₃)—(CH₂)₂—O—C(O)H |
| 558 | 2-CH₃ | NH—(CH₂)₂—OH |
| 559 | 2-CH₃ | NH—(CH₂)₂—NH₂ |
| 560 | 2-CH₃ | NH—(CH₂)₂—NHCH₃ |
| 561 | 2-CH₃ | NH—(CH₂)₂—N(CH₃)₂ |
| 562 | 2-CH₃ | NH—(CH₂)₂—O—C(O)H |
| 563 | 2-CH₃ | NH—(CH₂)₃—OH |
| 564 | 2-CH₃ | NH—(CH₂)₃—NH₂ |
| 565 | 2-CH₃ | NH—(CH₂)₃—NHCH₃ |
| 566 | 2-CH₃ | NH—(CH₂)₃—N(CH₃)₂ |
| 567 | 2-CH₃ | NH—(CH₂)₃—O—C(O)H |
| 568 | 2-CH₃ | NH—CH(CH₃)—CH₂—OH |
| 569 | 2-CH₃ | NH—CH(CH₃)—CH₂—NH₂ |
| 570 | 2-CH₃ | NH—CH(CH₃)—CH₂—NHCH₃ |
| 571 | 2-CH₃ | NH—CH(CH₃)—CH₂—N(CH₃)₂ |
| 572 | 2-CH₃ | NH—CH(CH₃)—CH₂—O—C(O)H |
| 573 | 2-CH₃ | NH—CH₂—CH(CH₃)—OH |
| 574 | 2-CH₃ | NH—CH₂—CH(CH₃)—NH₂ |
| 575 | 2-CH₃ | NH—CH₂—CH(CH₃)—NHCH₃ |
| 576 | 2-CH₃ | NH—CH₂—CH(CH₃)—N(CH₃)₂ |
| 577 | 2-CH₃ | NH—CH₂—CH(CH₃)—O—C(O)H |
| 578 | 2-CH₃ | NH—(CH₂)₂—O—(CH₂)₂—OH |
| 579 | 2-CH₃ | NH—(CH₂)₂—O—(CH₂)₂—NH₂ |
| 580 | 2-CH₃ | NH—(CH₂)₂—O—(CH₂)₂—NHCH₃ |
| 581 | 2-CH₃ | NH—(CH₂)₂—O—(CH₂)₂—N(CH₃)₂ |
| 582 | 2-CH₃ | NH—(CH₂)₂—O—(CH₂)₂—O—C(O)H |
| 583 | 2-CH₃ | NH—(CH₂)₂—NH—(CH₂)₂—OH |
| 584 | 2-CH₃ | NH—(CH₂)₂—NH—(CH₂)₂—NH₂ |
| 585 | 2-CH₃ | NH—(CH₂)₂—NH—(CH₂)₂—NHCH₃ |
| 586 | 2-CH₃ | NH—(CH₂)₂—NH—(CH₂)₂—N(CH₃)₂ |
| 587 | 2-CH₃ | NH—(CH₂)₂—NH—(CH₂)₂—O—C(O)H |
| 588 | 2-CH₃ | NH—(CH₂)₂—N(CH₃)—(CH₂)₂—OH |
| 589 | 2-CH₃ | NH—(CH₂)₂—N(CH₃)—(CH₂)₂—NH₂ |
| 590 | 2-CH₃ | NH—(CH₂)₂—N(CH₃)—(CH₂)₂—NHCH₃ |
| 591 | 2-CH₃ | NH—(CH₂)₂—N(CH₃)—(CH₂)₂—N(CH₃)₂ |
| 592 | 2-CH₃ | NH—(CH₂)₂—N(CH₃)—(CH₂)₂—O—C(O)H |
| 593 | 2-CH₃ | N(CH₃)—(CH₂)₂—OH |
| 594 | 2-CH₃ | N(CH₃)—(CH₂)₂—NH₂ |
| 595 | 2-CH₃ | N(CH₃)—(CH₂)₂—NHCH₃ |
| 596 | 2—CH₃ | N(CH₃)—(CH₂)₂—N(CH₃)₂ |
| 597 | 2—CH₃ | N(CH₃)—(CH₂)₂—O—C(O)H |
| 598 | 2—CH₃ | N(CH₃)—(CH₂)₃—OH |
| 599 | 2—CH₃ | N(CH₃)—(CH₂)₃—NH₂ |
| 600 | 2—CH₃ | N(CH₃)—(CH₂)₃—NHCH₃ |
| 601 | 2—CH₃ | N(CH₃)—(CH₂)₃—N(CH₃)₂ |
| 602 | 2—CH₃ | N(CH₃)—(CH₂)₃—O—C(O)H |
| 603 | 2—CH₃ | N(CH₃)—CH(CH₃)—CH₂—OH |
| 604 | 2—CH₃ | N(CH₃)—CH(CH₃)—CH₂—NH₂ |
| 605 | 2—CH₃ | N(CH₃)—CH(CH₃)—CH₂—NHCH₃ |
| 606 | 2—CH₃ | N(CH₃)—CH(CH₃)—CH₂—N(CH₃)₂ |
| 607 | 2—CH₃ | N(CH₃)—CH(CH₃)—CH₂—O—C(O)H |
| 608 | 2—CH₃ | N(CH₃)—CH₂—CH(CH₃)—OH |
| 609 | 2—CH₃ | N(CH₃)—CH₂—CH(CH₃)—NH₂ |
| 610 | 2—CH₃ | N(CH₃)—CH₂—CH(CH₃)—NHCH₃ |
| 611 | 2—CH₃ | N(CH₃)—CH₂—CH(CH₃)—N(CH₃)₂ |
| 612 | 2—CH₃ | N(CH₃)—CH₂—CH(CH₃)—O—C(O)H |
| 613 | 2—CH₃ | N(CH₃)—(CH₂)₂—O—(CH₂)₂—OH |
| 614 | 2—CH₃ | N(CH₃)—(CH₂)₂—O—(CH₂)₂—NH₂ |
| 615 | 2—CH₃ | N(CH₃)—(CH₂)₂—O—(CH₂)₂—NHCH₃ |
| 616 | 2—CH₃ | N(CH₃)—(CH₂)₂—O—(CH₂)₂—N(CH₃)₂ |
| 617 | 2—CH₃ | N(CH₃)—(CH₂)₂—O—(CH₂)₂—O—C(O)H |
| 618 | 2—CH₃ | N(CH₃)—(CH₂)₂—NH—(CH₂)₂—OH |
| 619 | 2—CH₃ | N(CH₃)—(CH₂)₂—NH—(CH₂)₂—NH₂ |
| 620 | 2—CH₃ | N(CH₃)—(CH₂)₂—NH—(CH₂)₂—NHCH₃ |
| 621 | 2—CH₃ | N(CH₃)—(CH₂)₂—NH—(CH₂)₂—N(CH₃)₂ |
| 622 | 2—CH₃ | N(CH₃)—(CH₂)₂—NH—(CH₂)₂—O—C(O)H |
| 623 | 2—CH₃ | N(CH₃)—(CH₂)₂—N(CH₃)—(CH₂)₂—OH |
| 624 | 2—CH₃ | N(CH₃)—(CH₂)₂—N(CH₃)—(CH₂)₂—NH₂ |
| 625 | 2—CH₃ | N(CH₃)—(CH₂)₂—N(CH₃)—(CH₂)₂—NHCH₃ |
| 626 | 2—CH₃ | N(CH₃)—(CH₂)₂—N(CH₃)—(CH₂)₂—N(CH₃)₂ |
| 627 | 2—CH₃ | N(CH₃)—(CH₂)₂—N(CH₃)—(CH₂)₂—O—C(O)H |
| 628 | 2,6-F₂ | C(O)NH—(CH₂)₂—OH |
| 629 | 2,6-F₂ | C(O)NH—(CH₂)₂—NH₂ |
| 630 | 2,6-F₂ | C(O)NH—(CH₂)₂—NHCH₃ |
| 631 | 2,6-F₂ | C(O)NH—(CH₂)₂—N(CH₃)₂ |
| 632 | 2,6-F₂ | C(O)NH—(CH₂)₂—O—C(O)H |
| 633 | 2,6-F₂ | C(O)NH—(CH₂)₃—OH |
| 634 | 2,6-F₂ | C(O)NH—(CH₂)₃—NH₂ |
| 635 | 2,6-F₂ | C(O)NH—(CH₂)₃—NHCH₃ |
| 636 | 2,6-F₂ | C(O)NH—(CH₂)₃—N(CH₃)₂ |
| 637 | 2,6-F₂ | C(O)NH—(CH₂)₃—O—C(O)H |
| 638 | 2,6-F₂ | C(O)NH—CH(CH₃)—CH₂—OH |
| 639 | 2,6-F₂ | C(O)NH—CH(CH₃)—CH₂—NH₂ |
| 640 | 2,6-F₂ | C(O)NH—CH(CH₃)—CH₂—NHCH₃ |
| 641 | 2,6-F₂ | C(O)NH—CH(CH₃)—CH₂—N(CH₃)₂ |
| 642 | 2,6-F₂ | C(O)NH—CH(CH₃)—CH₂—O—C(O)H |
| 643 | 2,6-F₂ | C(O)NH—CH₂—CH(CH₃)—OH |
| 644 | 2,6-F₂ | C(O)NH—CH₂—CH(CH₃)—NH₂ |
| 645 | 2,6-F₂ | C(O)NH—CH₂—CH(CH₃)—NHCH₃ |
| 646 | 2,6-F₂ | C(O)NH—CH₂—CH(CH₃)—N(CH₃)₂ |
| 647 | 2,6-F₂ | C(O)NH—CH₂—CH(CH₃)—O—C(O)H |
| 648 | 2,6-F₂ | C(O)NH—(CH₂)₂—O—(CH₂)₂—OH |
| 649 | 2,6-F₂ | C(O)NH—(CH₂)₂—O—(CH₂)₂—NH₂ |
| 650 | 2,6-F₂ | C(O)NH—(CH₂)₂—O—(CH₂)₂—NHCH₃ |
| 651 | 2,6-F₂ | C(O)NH—(CH₂)₂—O—(CH₂)₂—N(CH₃)₂ |
| 652 | 2,6-F₂ | C(O)NH—(CH₂)₂—O—(CH₂)₂—O—C(O)H |
| 653 | 2,6-F₂ | C(O)NH—(CH₂)₂—NH—(CH₂)₂—OH |
| 654 | 2,6-F₂ | C(O)NH—(CH₂)₂—NH—(CH₂)₂—NH₂ |
| 655 | 2,6-F₂ | C(O)NH—(CH₂)₂—NH—(CH₂)₂—NHCH₃ |
| 656 | 2,6-F₂ | C(O)NH—(CH₂)₂—NH—(CH₂)₂—N(CH₃)₂ |
| 657 | 2,6-F₂ | C(O)NH—(CH₂)₂—NH—(CH₂)₂—O—C(O)H |
| 658 | 2,6-F₂ | C(O)NH—(CH₂)₂—N(CH₃)—(CH₂)₂—OH |
| 659 | 2,6-F₂ | C(O)NH—(CH₂)₂—N(CH₃)—(CH₂)₂—NH₂ |
| 660 | 2,6-F₂ | C(O)NH—(CH₂)₂—N(CH₃)—(CH₂)₂—NHCH₃ |
| 661 | 2,6-F₂ | C(O)NH—(CH₂)₂—N(CH₃)—(CH₂)₂—N(CH₃)₂ |
| 662 | 2,6-F₂ | C(O)NH—(CH₂)₂—N(CH₃)—(CH₂)₂—O—C(O)H |
| 663 | 2,6-F₂ | C(O)N(CH₃)—(CH₂)₂—OH |
| 664 | 2,6-F₂ | C(O)N(CH₃)—(CH₂)₂—NH₂ |
| 665 | 2,6-F₂ | C(O)N(CH₃)—(CH₂)₂—NHCH₃ |
| 666 | 2,6-F₂ | C(O)N(CH₃)—(CH₂)₂—N(CH₃)₂ |
| 667 | 2,6-F₂ | C(O)N(CH₃)—(CH₂)₂—O—C(O)H |
| 668 | 2,6-F₂ | C(O)N(CH₃)—(CH₂)₃—OH |
| 669 | 2,6-F₂ | C(O)N(CH₃)—(CH₂)₃—NH₂ |
| 670 | 2,6-F₂ | C(O)N(CH₃)—(CH₂)₃—NHCH₃ |
| 671 | 2,6-F₂ | C(O)N(CH₃)—(CH₂)₃—N(CH₃)₂ |
| 672 | 2,6-F₂ | C(O)N(CH₃)—(CH₂)₃—O—C(O)H |
| 673 | 2,6-F₂ | C(O)N(CH₃)—CH(CH₃)—CH₂—OH |
| 674 | 2,6-F₂ | C(O)N(CH₃)—CH(CH₃)—CH₂—NH₂ |
| 675 | 2,6-F₂ | C(O)N(CH₃)—CH(CH₃)—CH₂—NHCH₃ |
| 676 | 2,6-F₂ | C(O)N(CH₃)—CH(CH₃)—CH₂—N(CH₃)₂ |
| 677 | 2,6-F₂ | C(O)N(CH₃)—CH(CH₃)—CH₂—O—C(O)H |
| 678 | 2,6-F₂ | C(O)N(CH₃)—CH₂—CH(CH₃)—OH |
| 679 | 2,6-F₂ | C(O)N(CH₃)—CH₂—CH(CH₃)—NH₂ |
| 680 | 2,6-F₂ | C(O)N(CH₃)—CH₂—CH(CH₃)—NHCH₃ |
| 681 | 2,6-F₂ | C(O)N(CH₃)—CH₂—CH(CH₃)—N(CH₃)₂ |
| 682 | 2,6-F₂ | C(O)N(CH₃)—(CH₂)₂—O—(CH₂)₂—OH |
| 683 | 2,6-F₂ | C(O)N(CH₃)—(CH₂)₂—O—(CH₂)₂—NH₂ |
| 684 | 2,6-F₂ | C(O)N(CH₃)—(CH₂)₂—O—(CH₂)₂—NHCH₃ |
| 685 | 2,6-F₂ | C(O)N(CH₃)—(CH₂)₂—O—(CH₂)₂—N(CH₃)₂ |
| 686 | 2,6-F₂ | C(O)N(CH₃)—(CH₂)₂—O—(CH₂)₂—O—C(O)H |
| 687 | 2,6-F₂ | C(O)N(CH₃)—(CH₂)₂—NH—(CH₂)₂—OH |
| 688 | 2,6-F₂ | C(O)N(CH₃)—(CH₂)₂—NH—(CH₂)₂—NH₂ |
| 689 | 2,6-F₂ | C(O)N(CH₃)—(CH₂)₂—NH—(CH₂)₂—NHCH₃ |
| 690 | 2,6-F₂ | C(O)N(CH₃)—(CH₂)₂—NH—(CH₂)₂—N(CH₃)₂ |
| 691 | 2,6-F₂ | C(O)N(CH₃)—(CH₂)₂—NH—(CH₂)₂—O—C(O)H |
| 692 | 2,6-F₂ | C(O)N(CH₃)—(CH₂)₂—N(CH₃)—(CH₂)₂—OH |
| 693 | 2,6-F₂ | C(O)N(CH₃)—(CH₂)₂—N(CH₃)—(CH₂)₂—NH₂ |
| 694 | 2,6-F₂ | C(O)N(CH₃)—(CH₂)₂—N(CH₃)—(CH₂)₂—NHCH₃ |
| 695 | 2,6-F₂ | C(O)N(CH₃)—(CH₂)₂—N(CH₃)—(CH₂)₂—N(CH₃)₂ |
| 696 | 2,6-F₂ | C(O)N(CH₃)—(CH₂)₂—N(CH₃)—(CH₂)₂—O—C(O)H |
| 697 | 2,6-F₂ | C(O)O—(CH₂)₂—OH |
| 698 | 2,6-F₂ | C(O)O—(CH₂)₂—NH₂ |
| 699 | 2,6-F₂ | C(O)O—(CH₂)₂—NHCH₃ |
| 700 | 2,6-F₂ | C(O)O—(CH₂)₂—N(CH₃)₂ |
| 701 | 2,6-F₂ | C(O)O—(CH₂)₂—O—C(O)H |
| 702 | 2,6-F₂ | C(O)O—(CH₂)₃—OH |
| 703 | 2,6-F₂ | C(O)O—(CH₂)₃—NH₂ |
| 704 | 2,6-F₂ | C(O)O—(CH₂)₃—NHCH₃ |
| 705 | 2,6-F₂ | C(O)O—(CH₂)₃—N(CH₃)₂ |
| 706 | 2,6-F₂ | C(O)O—(CH₂)₃—O—C(O)H |
| 707 | 2,6-F₂ | C(O)O—CH(CH₃)—CH₂—OH |
| 708 | 2,6-F₂ | C(O)O—CH(CH₃)—CH₂—NH₂ |
| 709 | 2,6-F₂ | C(O)O—CH(CH₃)—CH₂—NHCH₃ |
| 710 | 2,6-F₂ | C(O)O—CH(CH₃)—CH₂—N(CH₃)₂ |
| 711 | 2,6-F₂ | C(O)O—CH(CH₃)—CH₂—O—C(O)H |
| 712 | 2,6-F₂ | C(O)O—CH₂—CH(CH₃)—OH |
| 713 | 2,6-F₂ | C(O)O—CH₂—CH(CH₃)—NH₂ |
| 714 | 2,6-F₂ | C(O)O—CH₂—CH(CH₃)—NHCH₃ |
| 715 | 2,6-F₂ | C(O)O—CH₂—CH(CH₃)—N(CH₃)₂ |
| 716 | 2,6-F₂ | C(O)O—CH₂—CH(CH₃)—O—C(O)H |
| 717 | 2,6-F₂ | C(O)O—(CH₂)₂—O—(CH₂)₂—OH |
| 718 | 2,6-F₂ | C(O)O—(CH₂)₂—O—(CH₂)₂—NH₂ |
| 719 | 2,6-F₂ | C(O)O—(CH₂)₂—O—(CH₂)₂—NHCH₃ |
| 720 | 2,6-F₂ | C(O)O—(CH₂)₂—O—(CH₂)₂—N(CH₃)₂ |
| 721 | 2,6-F₂ | C(O)O—(CH₂)₂—O—(CH₂)₂—O—C(O)H |
| 722 | 2,6-F₂ | C(O)O—(CH₂)₂—NH—(CH₂)₂—OH |
| 723 | 2,6-F₂ | C(O)O—(CH₂)₂—NH—(CH₂)₂—NH₂ |
| 724 | 2,6-F₂ | C(O)O—(CH₂)₂—NH—(CH₂)₂—NHCH₃ |
| 725 | 2,6-F₂ | C(O)O—(CH₂)₂—NH—(CH₂)₂—N(CH₃)₂ |
| 726 | 2,6-F₂ | C(O)O—(CH₂)₂—NH—(CH₂)₂—O—C(O)H |
| 727 | 2,6-F₂ | C(O)O—(CH₂)₂—N(CH₃)—(CH₂)₂—OH |
| 728 | 2,6-F₂ | C(O)O—(CH₂)₂—N(CH₃)—(CH₂)₂—NH₂ |
| 729 | 2,6-F₂ | C(O)O—(CH₂)₂—N(CH₃)—(CH₂)₂—NHCH₃ |
| 730 | 2,6-F₂ | C(O)O—(CH₂)₂—N(CH₃)—(CH₂)₂—N(CH₃)₂ |
| 731 | 2,6-F₂ | C(O)O—(CH₂)₂—N(CH₃)—(CH₂)₂—O—C(O)H |
| 732 | 2,6-F₂ | O—(CH₂)₃—OCH₃ |
| 733 | 2,6-F₂ | O—(CH₂)₃-Piperidin-1-yl |
| 734 | 2,6-F₂ | O—(CH₂)₃-Piperazin-1-yl |
| 735 | 2,6-F₂ | O—(CH₂)₂-4-Methylpiperazinyl |
| 736 | 2,6-F₂ | Morpholin-1-yl |
| 737 | 2,6-F₂ | O—(CH₂)₃—OH |
| 738 | 2,6-F₂ | O—(CH₂)₃—NH₂ |
| 739 | 2,6-F₂ | O—(CH₂)₃—NHCH₃ |
| 740 | 2,6-F₂ | O—(CH₂)₃—N(CH₃)₂ |
| 741 | 2,6-F₂ | O—(CH₂)₃—O—C(O)H |
| 742 | 2,6-F₂ | O—CH(CH₃)—CH₂—OH |
| 743 | 2,6-F₂ | O—CH(CH₃)—CH₂—NH₂ |
| 744 | 2,6-F₂ | O—CH(CH₃)—CH₂—NHCH₃ |
| 745 | 2,6-F₂ | O—CH(CH₃)—CH₂—N(CH₃)₂ |
| 746 | 2,6-F₂ | O—CH(CH₃)—CH₂—O—C(O)H |
| 747 | 2,6-F₂ | O—CH₂—CH(CH₃)—OH |
| 748 | 2,6-F₂ | O—CH₂—CH(CH₃)—NH₂ |
| 749 | 2,6-F₂ | O—CH₂—CH(CH₃)—NHCH₃ |
| 750 | 2,6-F₂ | O—CH₂—CH(CH₃)—N(CH₃)₂ |
| 751 | 2,6-F₂ | O—CH₂—CH(CH₃)—O—C(O)H |
| 752 | 2,6-F₂ | O—(CH₂)₂—O—(CH₂)₂—OH |
| 753 | 2,6-F₂ | O—(CH₂)₂—O—(CH₂)₂—NH₂ |
| 754 | 2,6-F₂ | O—(CH₂)₂—O—(CH₂)₂—NHCH₃ |
| 755 | 2,6-F₂ | O—(CH₂)₂—O—(CH₂)₂—N(CH₃)₂ |
| 756 | 2,6-F₂ | O—(CH₂)₂—O—(CH₂)₂—O—C(O)H |
| 757 | 2,6-F₂ | O—(CH₂)₂—NH—(CH₂)₂—OH |
| 758 | 2,6-F₂ | O—(CH₂)₂—NH—(CH₂)₂—NH₂ |
| 759 | 2,6-F₂ | O—(CH₂)₂—NH—(CH₂)₂—NHCH₃ |
| 760 | 2,6-F₂ | O—(CH₂)₂—NH—(CH₂)₂—N(CH₃)₂ |
| 761 | 2,6-F₂ | O—(CH₂)₂—NH—(CH₂)₂—O—C(O)H |
| 762 | 2,6-F₂ | O—(CH₂)₂—N(CH₃)—(CH₂)₂—OH |
| 763 | 2,6-F₂ | O—(CH₂)₂—N(CH₃)—(CH₂)₂—NH₂ |
| 764 | 2,6-F₂ | O—(CH₂)₂—N(CH₃)—(CH₂)₂—NHCH₃ |
| 765 | 2,6-F₂ | O—(CH₂)₂—N(CH₃)—(CH₂)₂—N(CH₃)₂ |
| 766 | 2,6-F₂ | O—(CH₂)₂—N(CH₃)—(CH₂)₂—O—C(O)H |
| 767 | 2,6-F₂ | NH—(CH₂)₂—OH |
| 768 | 2,6-F₂ | NH—(CH₂)₂—NH₂ |
| 769 | 2,6-F₂ | NH—(CH₂)₂—NHCH₃ |
| 770 | 2,6-F₂ | NH—(CH₂)₂—N(CH₃)₂ |
| 771 | 2,6-F₂ | NH—(CH₂)₂—O—C(O)H |
| 772 | 2,6-F₂ | NH—(CH₂)₃—OH |
| 773 | 2,6-F₂ | NH—(CH₂)₃—NH₂ |
| 774 | 2,6-F₂ | NH—(CH₂)₃—NHCH₃ |
| 775 | 2,6-F₂ | NH—(CH₂)₃—N(CH₃)₂ |
| 776 | 2,6-F₂ | NH—(CH₂)₃—O—C(O)H |
| 777 | 2,6-F₂ | NH—CH(CH₃)—CH₂—OH |
| 778 | 2,6-F₂ | NH—CH(CH₃)—CH₂—NH₂ |
| 779 | 2,6-F₂ | NH—CH(CH₃)—CH₂—NHCH₃ |
| 780 | 2,6-F₂ | NH—CH(CH₃)—CH₂—N(CH₃)₂ |
| 781 | 2,6-F₂ | NH—CH(CH₃)—CH₂—O—C(O)H |
| 782 | 2,6-F₂ | NH—CH₂—CH(CH₃)—OH |
| 783 | 2,6-F₂ | NH—CH₂—CH(CH₃)—NH₂ |
| 784 | 2,6-F₂ | NH—CH₂—CH(CH₃)—NHCH₃ |
| 785 | 2,6-F₂ | NH—CH₂—CH(CH₃)—N(CH₃)₂ |
| 786 | 2,6-F₂ | NH—CH₂—CH(CH₃)—O—C(O)H |
| 787 | 2,6-F₂ | NH—(CH₂)₂—O—(CH₂)₂—OH |
| 788 | 2,6-F₂ | NH—(CH₂)₂—O—(CH₂)₂—NH₂ |
| 789 | 2,6-F₂ | NH—(CH₂)₂—O—(CH₂)₂—NHCH₃ |
| 790 | 2,6-F₂ | NH—(CH₂)₂—O—(CH₂)₂—N(CH₃)₂ |
| 791 | 2,6-F₂ | NH—(CH₂)₂—O—(CH₂)₂—O—C(O)H |
| 792 | 2,6-F₂ | NH—(CH₂)₂—NH—(CH₂)₂—OH |
| 793 | 2,6-F₂ | NH—(CH₂)₂—NH—(CH₂)₂—NH₂ |
| 794 | 2,6-F₂ | NH—(CH₂)₂—NH—(CH₂)₂—NHCH₃ |
| 795 | 2,6-F₂ | NH—(CH₂)₂—NH—(CH₂)₂—N(CH₃)₂ |
| 796 | 2,6-F₂ | NH—(CH₂)₂—NH—(CH₂)₂—O—C(O)H |
| 797 | 2,6-F₂ | NH—(CH₂)₂—N(CH₃)—(CH₂)₂—OH |
| 798 | 2,6-F₂ | NH—(CH₂)₂—N(CH₃)—(CH₂)₂—NH₂ |
| 799 | 2,6-F₂ | NH—(CH₂)₂—N(CH₃)—(CH₂)₂—NHCH₃ |
| 800 | 2,6-F₂ | NH—(CH₂)₂—N(CH₃)—(CH₂)₂—N(CH₃)₂ |
| 801 | 2,6-F₂ | NH—(CH₂)₂—N(CH₃)—(CH₂)₂—O—C(O)H |
| 802 | 2,6-F₂ | N(CH₃)—(CH₂)₂—OH |
| 803 | 2,6-F₂ | N(CH₃)—(CH₂)₂—NH₂ |
| 804 | 2,6-F₂ | N(CH₃)—(CH₂)₂—NHCH₃ |
| 805 | 2,6-F₂ | N(CH₃)—(CH₂)₂—N(CH₃)₂ |
| 806 | 2,6-F₂ | N(CH₃)—(CH₂)₂—O—C(O)H |
| 807 | 2,6-F₂ | N(CH₃)—(CH₂)₃—OH |
| 808 | 2,6-F₂ | N(CH₃)—(CH₂)₃—NH₂ |
| 809 | 2,6-F₂ | N(CH₃)—(CH₂)₃—NHCH₃ |
| 810 | 2,6-F₂ | N(CH₃)—(CH₂)₃—N(CH₃)₂ |
| 811 | 2,6-F₂ | N(CH₃)—CH(CH₂)₃—O—C(O)H |
| 812 | 2,6-F₂ | N(CH₃)—CH(CH₃)—CH₂—OH |
| 813 | 2,6-F₂ | N(CH₃)—CH(CH₃)—CH₂—NH₂ |
| 814 | 2,6-F₂ | N(CH₃)—CH(CH₃)—CH₂—NHCH₃ |
| 815 | 2,6-F₂ | N(CH₃)—CH(CH₃)—CH₂—N(CH₃)₂ |
| 816 | 2,6-F₂ | N(CH₃)—CH(CH₃)—CH₂—O—C(O)H |
| 817 | 2,6-F₂ | N(CH₃)—CH₂—CH(CH₃)—OH |
| 818 | 2,6-F₂ | N(CH₃)—CH₂—CH(CH₃)—NH₂ |
| 819 | 2,6-F₂ | N(CH₃)—CH₂—CH(CH₃)—NHCH₃ |
| 820 | 2,6-F₂ | N(CH₃)—CH₂—CH(CH₃)—N(CH₃)₂ |
| 821 | 2,6-F₂ | N(CH₃)—CH₂—CH(CH₃)—O—C(O)H |
| 822 | 2,6-F₂ | N(CH₃)—(CH₂)₂—O—(CH₂)₂—OH |
| 823 | 2,6-F₂ | N(CH₃)—(CH₂)₂—O—(CH₂)₂—NH₂ |
| 824 | 2,6-F₂ | N(CH₃)—(CH₂)₂—O—(CH₂)₂—NHCH₃ |
| 825 | 2,6-F₂ | N(CH₃)—(CH₂)₂—O—(CH₂)₂—N(CH₃)₂ |
| 826 | 2,6-F₂ | N(CH₃)—(CH₂)₂—O—(CH₂)₂—O—C(O)H |
| 827 | 2,6-F₂ | N(CH₃)—(CH₂)₂—NH—(CH₂)₂—OH |
| 828 | 2,6-F₂ | N(CH₃)—(CH₂)₂—NH—(CH₂)₂—NH₂ |
| 829 | 2,6-F₂ | N(CH₃)—(CH₂)₂—NH—(CH₂)₂—NHCH₃ |
| 830 | 2,6-F₂ | N(CH₃)—(CH₂)₂—NH—(CH₂)₂—N(CH₃)₂ |
| 831 | 2,6-F₂ | N(CH₃)—(CH₂)₂—NH—(CH₂)₂—O—C(O)H |
| 832 | 2,6-F₂ | N(CH₃)—(CH₂)₂—N(CH₃)—(CH₂)₂—OH |
| 833 | 2,6-F₂ | N(CH₃)—(CH₂)₂—N(CH₃)—(CH₂)₂—NH₂ |
| 834 | 2,6-F₂ | N(CH₃)—(CH₂)₂—N(CH₃)—(CH₂)₂—NHCH₃ |
| 835 | 2,6-F₂ | N(CH₃)—(CH₂)₂—N(CH₃)—CH(CH₂)₂—N(CH₃)₂ |
| 836 | 2,6-F₂ | N(CH₃)—(CH₂)₂—N(CH₃)—(CH₂)₂—O—C(O)H |
| 837 | 2-F,6-Cl | C(O)NH—(CH₂)₂—OH |
| 838 | 2-F,6-Cl | C(O)NH—(CH₂)₂—NH₂ |
| 839 | 2-F,6-Cl | C(O)NH—(CH₂)₂—NHCH₃ |
| 840 | 2-F,6-Cl | C(O)NH—(CH₂)₂—N(CH₃)₂ |
| 841 | 2-F,6-Cl | C(O)NH—(CH₂)₂—O—C(O)H |
| 842 | 2-F,6-Cl | C(O)NH—(CH₂)₃—OH |
| 843 | 2-F,6-Cl | C(O)NH—(CH₂)₃—NH₂ |
| 844 | 2-F,6-Cl | C(O)NH—(CH₂)₃—NHCH₃ |
| 845 | 2-F,6-Cl | C(O)NH—(CH₂)₃—N(CH₃)₂ |
| 846 | 2-F,6-Cl | C(O)NH—(CH₂)₃—O—C(O)H |
| 847 | 2-F,6-Cl | C(O)NH—CH(CH₃)—CH₂—OH |
| 848 | 2-F,6-Cl | C(O)NH—CH(CH₃)—CH₂—NH₂ |
| 849 | 2-F,6-Cl | C(O)NH—CH(CH₃)—CH₂—NHCH₃ |
| 850 | 2-F,6-Cl | C(O)NH—CH(CH₃)—CH₂—N(CH₃)₂ |
| 851 | 2-F,6-Cl | C(O)NH—CH(CH₃)—CH₂—O—C(O)H |
| 852 | 2-F,6-Cl | C(O)NH—CH₂—CH(CH₃)—OH |
| 853 | 2-F,6-Cl | C(O)NH—CH₂—CH(CH₃)—NH₂ |
| 854 | 2-F,6-Cl | C(O)NH—CH₂—CH(CH₃)—NHCH₃ |
| 855 | 2-F,6-Cl | C(O)NH—CH₂—CH(CH₃)—N(CH₃)₂ |
| 856 | 2-F,6-Cl | C(O)NH—CH₂—CH(CH₃)—O—C(O)H |
| 857 | 2-F,6-Cl | C(O)NH—(CH₂)₂—O—(CH₂)₂—OH |
| 858 | 2-F,6-Cl | C(O)NH—(CH₂)₂—O—(CH₂)₂—NH₂ |
| 859 | 2-F,6-Cl | C(O)NH—(CH₂)₂—O—(CH₂)₂—NHCH₃ |
| 860 | 2-F,6-Cl | C(O)NH—(CH₂)₂—O—(CH₂)₂—N(CH₃)₂ |
| 861 | 2-F,6-Cl | C(O)NH—(CH₂)₂—O—(CH₂)₂—O—C(O)H |
| 862 | 2-F,6-Cl | C(O)NH—(CH₂)₂—NH—(CH₂)₂—OH |
| 863 | 2-F,6-Cl | C(O)NH—(CH₂)₂—NH—(CH₂)₂—NH₂ |
| 864 | 2-F,6-Cl | C(O)NH—(CH₂)₂—NH—(CH₂)₂—NHCH₃ |
| 865 | 2-F,6-Cl | C(O)NH—(CH₂)₂—NH—(CH₂)₂—N(CH₃)₂ |
| 866 | 2-F,6-Cl | C(O)NH—(CH₂)₂—NH—(CH₂)₂—O—C(O)H |
| 867 | 2-F,6-Cl | C(O)NH—(CH₂)₂—N(CH₃)—(CH₂)₂—OH |
| 868 | 2-F,6-Cl | C(O)NH—(CH₂)₂—N(CH₃)—(CH₂)₂—NH₂ |
| 869 | 2-F,6-Cl | C(O)NH—(CH₂)₂—N(CH₃)—(CH₂)₂—NHCH₃ |
| 870 | 2-F,6-Cl | C(O)NH—(CH₂)₂—N(CH₃)—(CH₂)₂—N(CH₃)₂ |
| 871 | 2-F,6-Cl | C(O)NH—(CH₂)₂—N(CH₃)—(CH₂)₂—O—C(O)H |
| 872 | 2-F,6-Cl | C(O)N(CH₃)—(CH₂)₂—OH |
| 873 | 2-F,6-Cl | C(O)N(CH₃)—(CH₂)₂—NH₂ |
| 874 | 2-F,6-Cl | C(O)N(CH₃)—(CH₂)₂—NHCH₃ |
| 875 | 2-F,6-Cl | C(O)N(CH₃)—(CH₂)₂—N(CH₃)₂ |
| 876 | 2-F,6-Cl | C(O)N(CH₃)—(CH₂)₂—O—C(O)H |
| 877 | 2-F,6-Cl | C(O)N(CH₃)—(CH₂)₃—OH |
| 878 | 2-F,6-Cl | C(O)N(CH₃)—(CH₂)₃—NH₂ |
| 879 | 2-F,6-Cl | C(O)N(CH₃)—(CH₂)₃—NHCH₃ |
| 880 | 2-F,6-Cl | C(O)N(CH₃)—(CH₂)₃—N(CH₃)₂ |
| 881 | 2-F,6-Cl | C(O)N(CH₃)—(CH₂)₃—O—C(O)H |
| 882 | 2-F,6-Cl | C(O)N(CH₃)—CH(CH₃)—CH₂—OH |
| 883 | 2-F,6-Cl | C(O)N(CH₃)—CH(CH₃)—CH₂—NH₂ |
| 884 | 2-F,6-Cl | C(O)N(CH₃)—CH(CH₃)—CH₂—NHCH₃ |
| 885 | 2-F,6-Cl | C(O)N(CH₃)—CH(CH₃)—CH₂—N(CH₃)₂ |
| 886 | 2-F,6-Cl | C(O)N(CH₃)—CH(CH₃)—CH₂—O—C(O)H |
| 887 | 2-F,6-Cl | C(O)N(CH₃)—CH₂—CH(CH₃)—OH |
| 888 | 2-F,6-Cl | C(O)N(CH₃)—CH₂—CH(CH₃)—NH₂ |
| 889 | 2-F,6-Cl | C(O)N(CH₃)—CH₂—CH(CH₃)—NHCH₃ |
| 890 | 2-F,6-Cl | C(O)N(CH₃)—CH₂—CH(CH₃)—N(CH₃)₂ |
| 891 | 2-F,6-Cl | C(O)N(CH₃)—(CH₂)₂—O—(CH₂)₂—OH |
| 892 | 2-F,6-Cl | C(O)N(CH₃)—(CH₂)₂—O—(CH₂)₂—NH₂ |
| 893 | 2-F,6-Cl | C(O)N(CH₃)—(CH₂)₂—O—(CH₂)₂—NHCH₃ |
| 894 | 2-F,6-Cl | C(O)N(CH₃)—(CH₂)₂—O—(CH₂)₂—N(CH₃)₂ |
| 895 | 2-F,6-Cl | C(O)N(CH₃)—(CH₂)₂—O—(CH₂)₂—O—C(O)H |
| 896 | 2-F,6-Cl | C(O)N(CH₃)—(CH₂)₂—NH—(CH₂)₂—OH |
| 897 | 2-F,6-Cl | C(O)N(CH₃)—(CH₂)₂—NH—(CH₂)₂—NH₂ |
| 898 | 2-F,6-Cl | C(O)N(CH₃)—(CH₂)₂—NH—(CH₂)₂—NHCH₃ |
| 899 | 2-F,6-Cl | C(O)N(CH₃)—(CH₂)₂—NH—(CH₂)₂—N(CH₃)₂ |
| 900 | 2-F,6-Cl | C(O)N(CH₃)—(CH₂)₂—NH—(CH₂)₂—O—C(O)H |
| 901 | 2-F,6-Cl | C(O)N(CH₃)—(CH₂)₂—N(CH₃)—(CH₂)₂—OH |
| 902 | 2-F,6-Cl | C(O)N(CH₃)—(CH₂)₂—N(CH₃)—(CH₂)₂—NH₂ |
| 903 | 2-F,6-Cl | C(O)N(CH₃)—(CH₂)₂—N(CH₃)—(CH₂)₂—NHCH₃ |
| 904 | 2-F,6-Cl | C(O)N(CH₃)—(CH₂)₂—N(CH₃)—(CH₂)₂—N(CH₃)₂ |
| 905 | 2-F,6-Cl | C(O)N(CH₃)—(CH₂)₂—N(CH₃)—(CH₂)₂—O—C(O)H |
| 906 | 2-F,6-Cl | C(O)O—(CH₂)₂—OH |
| 907 | 2-F,6-Cl | C(O)O—(CH₂)₂—NH₂ |
| 908 | 2-F,6-Cl | C(O)O—(CH₂)₂—NHCH₃ |
| 909 | 2-F,6-Cl | C(O)O—(CH₂)₂—N(CH₃)₂ |
| 910 | 2-F,6-Cl | C(O)O—(CH₂)₂—O—C(O)H |
| 911 | 2-F,6-Cl | C(O)O—(CH₂)₃—OH |
| 912 | 2-F,6-Cl | C(O)O—(CH₂)₃—NH₂ |
| 913 | 2-F,6-Cl | C(O)O—(CH₂)₃—NHCH₃ |
| 914 | 2-F,6-Cl | C(O)O—(CH₂)₃—N(CH₃)₂ |
| 915 | 2-F,6-Cl | C(O)O—(CH₂)₃—O—C(O)H |
| 916 | 2-F,6-Cl | C(O)O—CH(CH₃)—CH₂—OH |
| 917 | 2-F,6-Cl | C(O)O—CH(CH₃)—CH₂—NH₂ |
| 918 | 2-F,6-Cl | C(O)O—CH(CH₃)—CH₂—NHCH₃ |
| 919 | 2-F,6-Cl | C(O)O—CH(CH₃)—CH₂—N(CH₃)₂ |
| 920 | 2-F,6-Cl | C(O)O—CH(CH₃)—CH₂—O—C(O)H |
| 921 | 2-F,6-Cl | C(O)O—CH₂—CH(CH₃)—OH |
| 922 | 2-F,6-Cl | C(O)O—CH₂—CH(CH₃)—NH₂ |
| 923 | 2-F,6-Cl | C(O)O—CH₂—CH(CH₃)—NHCH₃ |
| 924 | 2-F,6-Cl | C(O)O—CH₂—CH(CH₃)—N(CH₃)₂ |
| 925 | 2-F,6-Cl | C(O)O—CH₂—CH(CH₃)—O—C(O)H |
| 926 | 2-F,6-Cl | C(O)O—(CH₂)₂—O—(CH₂)₂—OH |
| 927 | 2-F,6-Cl | C(O)O—(CH₂)₂—O—(CH₂)₂—NH₂ |
| 928 | 2-F,6-Cl | C(O)O—(CH₂)₂—O—(CH₂)₂—NHCH₃ |
| 929 | 2-F,6-Cl | C(O)O—(CH₂)₂—O—(CH₂)₂—N(CH₃)₂ |
| 930 | 2-F,6-Cl | C(O)O—(CH₂)₂—O—(CH₂)₂—O—C(O)H |
| 931 | 2-F,6-Cl | C(O)O—(CH₂)₂—NH—(CH₂)₂—OH |
| 932 | 2-F,6-Cl | C(O)O—(CH₂)₂—NH—(CH₂)₂—NH₂ |
| 933 | 2-F,6-Cl | C(O)O—(CH₂)₂—NH—(CH₂)₂—NHCH₃ |
| 934 | 2-F,6-Cl | C(O)O—(CH₂)₂—NH—(CH₂)₂—N(CH₃)₂ |
| 935 | 2-F,6-Cl | C(O)O—(CH₂)₂—NH—(CH₂)₂—O—C(O)H |
| 936 | 2-F,6-Cl | C(O)O—(CH₂)₂—N(CH₃)—(CH₂)₂—OH |
| 937 | 2-F,6-Cl | C(O)O—(CH₂)₂—N(CH₃)—(CH₂)₂—NH₂ |
| 938 | 2-F,6-Cl | C(O)O—(CH₂)₂—N(CH₃)—(CH₂)₂—NHCH₃ |
| 939 | 2-F,6-Cl | C(O)O—(CH₂)₂—N(CH₃)—(CH₂)₂—N(CH₃)₂ |
| 940 | 2-F,6-Cl | C(O)O—(CH₂)₂—N(CH₃)—CH(CH₂)₂—O—C(O)H |
| 941 | 2-F,6-Cl | O—(CH₂)₃—OCH₃ |
| 942 | 2-F,6-Cl | O—(CH₂)₃-Piperidin-1-yl |
| 943 | 2-F,6-Cl | O—(CH₂)₃-Piperazin-1-yl |
| 944 | 2-F,6-Cl | O—(CH₂)₂-4-Methylpiperazinyl |
| 945 | 2-F,6-Cl | Morpholin-1-yl |
| 946 | 2-F,6-Cl | O—(CH₂)₃—OH |
| 947 | 2-F,6-Cl | O—(CH₂)₃—NH₂ |
| 948 | 2-F,6-Cl | O—(CH₂)₃—NHCH₃ |
| 949 | 2-F,6-Cl | O—(CH₂)₃—N(CH₃)₂ |
| 950 | 2-F,6-Cl | O—(CH₂)₃—O—C(O)H |
| 951 | 2-F,6-Cl | O—CH(CH₃)—CH₂—OH |
| 952 | 2-F,6-Cl | O—CH(CH₃)—CH₂—NH₂ |
| 953 | 2-F,6-Cl | O—CH(CH₃)—CH₂—NHCH₃ |
| 954 | 2-F,6-Cl | O—CH(CH₃)—CH₂—N(CH₃)₂ |
| 955 | 2-F,6-Cl | O—CH(CH₃)—CH₂—O—C(O)H |
| 956 | 2-F,6-Cl | O—CH₂—CH(CH₃)—OH |
| 957 | 2-F,6-Cl | O—CH₂—CH(CH₃)—NH₂ |
| 958 | 2-F,6-Cl | O—CH₂—CH(CH₃)—NHCH₃ |
| 959 | 2-F,6-Cl | O—CH₂—CH(CH₃)—N(CH₃)₂ |
| 960 | 2-F,6-Cl | O—CH₂—CH(CH₃)—O—C(O)H |
| 961 | 2-F,6-Cl | O—(CH₂)₂—O—(CH₂)₂—OH |
| 962 | 2-F,6-Cl | O—(CH₂)₂—O—(CH₂)₂—NH₂ |
| 963 | 2-F,6-Cl | O—(CH₂)₂—O—(CH₂)₂—NHCH₃ |
| 964 | 2-F,6-Cl | O—(CH₂)₂—O—(CH₂)₂—N(CH₃)₂ |
| 965 | 2-F,6-Cl | O—(CH₂)₂—O—(CH₂)₂—O—C(O)H |
| 966 | 2-F,6-Cl | O—(CH₂)₂—NH—(CH₂)₂—OH |
| 967 | 2-F,6-Cl | O—(CH₂)₂—NH—(CH₂)₂—NH₂ |
| 968 | 2-F,6-Cl | O—(CH₂)₂—NH—(CH₂)₂—NHCH₃ |
| 969 | 2-F,6-Cl | O—(CH₂)₂—NH—(CH₂)₂—N(CH₃)₂ |
| 970 | 2-F,6-Cl | O—(CH₂)₂—NH—(CH₂)₂—O—C(O)H |
| 971 | 2-F,6-Cl | O—(CH₂)₂—N(CH₃)—(CH₂)₂—OH |
| 972 | 2-F,6-Cl | O—(CH₂)₂—N(CH₃)—(CH₂)₂—NH₂ |
| 973 | 2-F,6-Cl | O—(CH₂)₂—N(CH₃)—(CH₂)₂—NHCH₃ |
| 974 | 2-F,6-Cl | O—(CH₂)₂—N(CH₃)—(CH₂)₂—N(CH₃)₂ |
| 975 | 2-F,6-Cl | O—(CH₂)₂—N(CH₃)—(CH₂)₂—O—C(O)H |
| 976 | 2-F,6-Cl | NH—(CH₂)₂—OH |
| 977 | 2-F,6-Cl | NH—(CH₂)₂—NH₂ |
| 978 | 2-F,6-Cl | NH—(CH₂)₂—NHCH₃ |
| 979 | 2-F,6-Cl | NH—(CH₂)₂—N(CH₃)₂ |
| 980 | 2-F,6-Cl | NH—(CH₂)₂—O—C(O)H |
| 981 | 2-F,6-Cl | NH—(CH₂)₃—OH |
| 982 | 2-F,6-Cl | NH—(CH₂)₃—NH₂ |
| 983 | 2-F,6-Cl | NH—(CH₂)₃—NHCH₃ |
| 984 | 2-F,6-Cl | NH—(CH₂)₃—N(CH₃)₂ |
| 985 | 2-F,6-Cl | NH—(CH₂)₃—O—C(O)H |
| 986 | 2-F,6-Cl | NH—CH(CH₃)—CH₂—OH |
| 987 | 2-F,6-Cl | NH—CH(CH₃)—CH₂—NH₂ |
| 988 | 2-F,6-Cl | NH—CH(CH₃)—CH₂—NHCH₃ |
| 989 | 2-F,6-Cl | NH—CH(CH₃)—CH₂—N(CH₃)₂ |
| 990 | 2-F,6-Cl | NH—CH(CH₃)—CH₂—O—C(O)H |
| 991 | 2-F,6-Cl | NH—CH₂—CH(CH₃)—OH |
| 992 | 2-F,6-Cl | NH—CH₂—CH(CH₃)—NH₂ |
| 993 | 2-F,6-Cl | NH—CH₂—CH(CH₃)—NHCH₃ |
| 994 | 2-F,6-Cl | NH—CH₂—CH(CH₃)—N(CH₃)₂ |
| 995 | 2-F,6-Cl | NH—CH₂—CH(CH₃)—O—C(O)H |
| 996 | 2-F,6-Cl | NH—(CH₂)₂—O—(CH₂)₂—OH |
| 997 | 2-F,6-Cl | NH—(CH₂)₂—O—(CH₂)₂—NH₂ |
| 998 | 2-F,6-Cl | NH—(CH₂)₂—O—(CH₂)₂—NHCH₃ |
| 999 | 2-F,6-Cl | NH—(CH₂)₂—O—(CH₂)₂—N(CH₃)₂ |
| 1000 | 2-F,6-Cl | NH—(CH₂)₂—O—(CH₂)₂—O—C(O)H |
| 1001 | 2-F,6-Cl | NH—(CH₂)₂—NH—(CH₂)₂—OH |
| 1002 | 2-F,6-Cl | NH—(CH₂)₂—NH—(CH₂)₂—NH₂ |
| 1003 | 2-F,6-Cl | NH—(CH₂)₂—NH—(CH₂)₂—NHCH₃ |
| 1004 | 2-F,6-Cl | NH—(CH₂)₂—NH—(CH₂)₂—N(CH₃)₂ |
| 1005 | 2-F,6-Cl | NH—(CH₂)₂—NH—(CH₂)₂—O—C(O)H |
| 1006 | 2-F,6-Cl | NH—(CH₂)₂—N(CH₃)—(CH₂)₂—OH |
| 1007 | 2-F,6-Cl | NH—(CH₂)₂—N(CH₃)—(CH₂)₂—NH₂ |
| 1008 | 2-F,6-Cl | NH—(CH₂)₂—N(CH₃)—(CH₂)₂—NHCH₃ |
| 1009 | 2-F,6-Cl | NH—(CH₂)₂—N(CH₃)—(CH₂)₂—N(CH₃)₂ |
| 1010 | 2-F,6-Cl | NH—(CH₂)₂—N(CH₃)—(CH₂)₂—O—C(O)H |
| 1011 | 2-F,6-Cl | N(CH₃)—(CH₂)₂—OH |
| 1012 | 2-F,6-Cl | N(CH₃)—(CH₂)₂—NH₂ |
| 1013 | 2-F,6-Cl | N(CH₃)—(CH₂)₂—NHCH₃ |
| 1014 | 2-F,6-Cl | N(CH₃)—(CH₂)₂—N(CH₃)₂ |
| 1015 | 2-F,6-Cl | N(CH₃)—(CH₂)₂—O—C(O)H |
| 1016 | 2-F,6-Cl | N(CH₃)—(CH₂)₃—OH |
| 1017 | 2-F,6-Cl | N(CH₃)—(CH₂)₃—NH₂ |
| 1018 | 2-F,6-Cl | N(CH₃)—(CH₂)₃—NHCH₃ |
| 1019 | 2-F,6-Cl | N(CH₃)—(CH₂)₃—N(CH₃)₂ |
| 1020 | 2-F,6-Cl | N(CH₃)—(CH₂)₃—O—C(O)H |
| 1021 | 2-F,6-Cl | N(CH₃)—CH(CH₃)—CH₂—OH |
| 1022 | 2-F,6-Cl | N(CH₃)—CH(CH₃)—CH₂—NH₂ |
| 1023 | 2-F,6-Cl | N(CH₃)—CH(CH₃)—CH₂—NHCH₃ |
| 1024 | 2-F,6-Cl | N(CH₃)—CH(CH₃)—CH₂—N(CH₃)₂ |
| 1025 | 2-F,6-Cl | N(CH₃)—CH(CH₃)—CH₂—O—C(O)H |
| 1026 | 2-F,6-Cl | N(CH₃)—CH₂—CH(CH₃)—OH |
| 1027 | 2-F,6-Cl | N(CH₃)—CH₂—CH(CH₃)—NH₂ |
| 1028 | 2-F,6-Cl | N(CH₃)—CH₂—CH(CH₃)—NHCH₃ |
| 1029 | 2-F,6-Cl | N(CH₃)—CH₂—CH(CH₃)—N(CH₃)₂ |
| 1030 | 2-F,6-Cl | N(CH₃)—CH₂—CH(CH₃)—O—C(O)H |
| 1031 | 2-F,6-Cl | N(CH₃)—(CH₂)₂—O—(CH₂)₂—OH |
| 1032 | 2-F,6-Cl | N(CH₃)—(CH₂)₂—O—(CH₂)₂—NH₂ |
| 1033 | 2-F,6-Cl | N(CH₃)—(CH₂)₂—O—(CH₂)₂—NHCH₃ |
| 1034 | 2-F,6-Cl | N(CH₃)—(CH₂)₂—O—(CH₂)₂—N(CH₃)₂ |
| 1035 | 2-F,6-Cl | N(CH₃)—(CH₂)₂—O—(CH₂)₂—O—C(O)H |
| 1036 | 2-F,6-Cl | N(CH₃)—(CH₂)₂—NH—(CH₂)₂—OH |
| 1037 | 2-F,6-Cl | N(CH₃)—(CH₂)₂—NH—(CH₂)₂—NH₂ |
| 1038 | 2-F,6-Cl | N(CH₃)—(CH₂)₂—NH—(CH₂)₂—NHCH₃ |
| 1039 | 2-F,6-Cl | N(CH₃)—(CH₂)₂—NH—(CH₂)₂—N(CH₃)₂ |
| 1040 | 2-F,6-Cl | N(CH₃)—(CH₂)₂—NH—(CH₂)₂—O—C(O)H |
| 1041 | 2-F,6-Cl | N(CH₃)—(CH₂)₂—N(CH₃)—(CH₂)₂—OH |
| 1042 | 2-F,6-Cl | N(CH₃)—(CH₂)₂—N(CH₃)—(CH₂)₂—NH₂ |
| 1043 | 2-F,6-Cl | N(CH₃)—(CH₂)₂—N(CH₃)—(CH₂)₂—NHCH₃ |
| 1044 | 2-F,6-Cl | N(CH₃)—(CH₂)₂—N(CH₃)—(CH₂)₂—N(CH₃)₂ |
| 1045 | 2-F,6-Cl | N(CH₃)—(CH₂)₂—N(CH₃)—(CH₂)₂—O—C(O)H |
| 1046 | 2-F,6-CH₃ | C(O)NH—(CH₂)₂—OH |
| 1047 | 2-F,6-CH₃ | C(O)NH—(CH₂)₂—NH₂ |
| 1048 | 2-F,6-CH₃ | C(O)NH—(CH₂)₂—NHCH₃ |
| 1049 | 2-F,6-CH₃ | C(O)NH—(CH₂)₂—N(CH₃)₂ |
| 1050 | 2-F,6-CH₃ | C(O)NH—(CH₂)₂—O—C(O)H |
| 1051 | 2-F,6-CH₃ | C(O)NH—(CH₂)₃—OH |
| 1052 | 2-F,6-CH₃ | C(O)NH—(CH₂)₃—NH₂ |
| 1053 | 2-F,6-CH₃ | C(O)NH—(CH₂)₃—NHCH₃ |
| 1054 | 2-F,6-CH₃ | C(O)NH—(CH₂)₃—N(CH₃)₂ |
| 1055 | 2-F,6-CH₃ | C(O)NH—(CH₂)₃—O—C(O)H |
| 1056 | 2-F,6-CH₃ | C(O)NH—CH(CH₃)—CH₂—OH |
| 1057 | 2-F,6-CH₃ | C(O)NH—CH(CH₃)—CH₂—NH₂ |
| 1058 | 2-F,6-CH₃ | C(O)NH—CH(CH₃)—CH₂—NHCH₃ |
| 1059 | 2-F,6-CH₃ | C(O)NH—CH(CH₃)—CH₂—N(CH₃)₂ |
| 1060 | 2-F,6-CH₃ | C(O)NH—CH(CH₃)—CH₂—O—C(O)H |
| 1061 | 2-F,6-CH₃ | C(O)NH—CH₂—CH(CH₃)—OH |
| 1062 | 2-F,6-CH₃ | C(O)NH—CH₂—CH(CH₃)—NH₂ |
| 1063 | 2-F,6-CH₃ | C(O)NH—CH₂—CH(CH₃)—NHCH₃ |
| 1064 | 2-F,6-CH₃ | C(O)NH—CH₂—CH(CH₃)—N(CH₃)₂ |
| 1065 | 2-F,6-CH₃ | C(O)NH—CH₂—CH(CH₃)—O—C(O)H |
| 1066 | 2-F,6-CH₃ | C(O)NH—(CH₂)₂—O—(CH₂)₂—OH |
| 1067 | 2-F,6-CH₃ | C(O)NH—(CH₂)₂—O—(CH₂)₂—NH₂ |
| 1068 | 2-F,6-CH₃ | C(O)NH—(CH₂)₂—O—(CH₂)₂—NHCH₃ |
| 1069 | 2-F,6-CH₃ | C(O)NH—(CH₂)₂—O—(CH₂)₂—N(CH₃)₂ |
| 1070 | 2-F,6-CH₃ | C(O)NH—(CH₂)₂—O—(CH₂)₂—O—C(O)H |
| 1071 | 2-F,6-CH₃ | C(O)NH—(CH₂)₂—NH—(CH₂)₂—OH |
| 1072 | 2-F,6-CH₃ | C(O)NH—(CH₂)₂—NH—(CH₂)₂—NH₂ |
| 1073 | 2-F,6-CH₃ | C(O)NH—(CH₂)₂—NH—(CH₂)₂—NHCH₃ |
| 1074 | 2-F,6-CH₃ | C(O)NH—(CH₂)₂—NH—(CH₂)₂—N(CH₃)₂ |
| 1075 | 2-F,6-CH₃ | C(O)NH—(CH₂)₂—NH—(CH₂)₂—O—C(O)H |
| 1076 | 2-F,6-CH₃ | C(O)NH—(CH₂)₂—N(CH₃)—(CH₂)₂—OH |
| 1077 | 2-F,6-CH₃ | C(O)NH—(CH₂)₂—N(CH₃)—(CH₂)₂—NH₂ |
| 1078 | 2-F,6-CH₃ | C(O)NH—(CH₂)₂—N(CH₃)—(CH₂)₂—NHCH₃ |
| 1079 | 2-F,6-CH₃ | C(O)NH—(CH₂)₂—N(CH₃)—(CH₂)₂—N(CH₃)₂ |
| 1080 | 2-F,6-CH₃ | C(O)NH—(CH₂)₂—N(CH₃)—(CH₂)₂—O—C(O)H |
| 1081 | 2-F,6-CH₃ | C(O)N(CH₃)—(CH₂)₂—OH |
| 1082 | 2-F,6-CH₃ | C(O)N(CH₃)—(CH₂)₂—NH₂ |
| 1083 | 2-F,6-CH₃ | C(O)N(CH₃)—(CH₂)₂—NHCH₃ |
| 1084 | 2-F,6-CH₃ | C(O)N(CH₃)—(CH₂)₂—N(CH₃)₂ |
| 1085 | 2-F,6-CH₃ | C(O)N(CH₃)—(CH₂)₂—O—C(O)H |
| 1086 | 2-F,6-CH₃ | C(O)N(CH₃)—(CH₂)₃—OH |
| 1087 | 2-F,6-CH₃ | C(O)N(CH₃)—(CH₂)₃—NH₂ |
| 1088 | 2-F,6-CH₃ | C(O)N(CH₃)—(CH₂)₃—NHCH₃ |
| 1089 | 2-F,6-CH₃ | C(O)N(CH₃)—(CH₂)₃—N(CH₃)₂ |
| 1090 | 2-F,6-CH₃ | C(O)N(CH₃)—(CH₂)₃—O—C(O)H |
| 1091 | 2-F,6-CH₃ | C(O)N(CH₃)—CH(CH₃)—CH₂—OH |
| 1092 | 2-F,6-CH₃ | C(O)N(CH₃)—CH(CH₃)—CH₂—NH₂ |
| 1093 | 2-F,6-CH₃ | C(O)N(CH₃)—CH(CH₃)—CH₂—NHCH₃ |
| 1094 | 2-F,6-CH₃ | C(O)N(CH₃)—CH(CH₃)—CH₂—N(CH₃)₂ |
| 1095 | 2-F,6-CH₃ | C(O)N(CH₃)—CH(CH₃)—CH₂—O—C(O)H |
| 1096 | 2-F,6-CH₃ | C(O)N(CH₃)—CH₂—CH(CH₃)—OH |
| 1097 | 2-F,6-CH₃ | C(O)N(CH₃)—CH₂—CH(CH₃)—NH₂ |
| 1098 | 2-F,6-CH₃ | C(O)N(CH₃)—CH₂—CH(CH₃)—NHCH₃ |
| 1099 | 2-F,6-CH₃ | C(O)N(CH₃)—CH₂—CH(CH₃)—N(CH₃)₂ |
| 1100 | 2-F,6-CH₃ | C(O)N(CH₃)—(CH₂)₂—O—(CH₂)₂—OH |
| 1101 | 2-F,6-CH₃ | C(O)N(CH₃)—(CH₂)₂—O—(CH₂)₂—NH₂ |
| 1102 | 2-F,6-CH₃ | C(O)N(CH₃)—(CH₂)₂—O—(CH₂)₂—NHCH₃ |
| 1103 | 2-F,6-CH₃ | C(O)N(CH₃)—(CH₂)₂—O—(CH₂)₂—N(CH₃)₂ |
| 1104 | 2-F,6-CH₃ | C(O)N(CH₃)—(CH₂)₂—O—(CH₂)₂—O—C(O)H |
| 1105 | 2-F,6-CH₃ | C(O)N(CH₃)—(CH₂)₂—NH—(CH₂)₂—OH |
| 1106 | 2-F,6-CH₃ | C(O)N(CH₃)—(CH₂)₂—NH—(CH₂)₂—NH₂ |
| 1107 | 2-F,6-CH₃ | C(O)N(CH₃)—(CH₂)₂—NH—(CH₂)₂—NHCH₃ |
| 1108 | 2-F,6-CH₃ | C(O)N(CH₃)—(CH₂)₂—NH—(CH₂)₂—N(CH₃)₂ |
| 1109 | 2-F,6-CH₃ | C(O)N(CH₃)—(CH₂)₂—NH—(CH₂)₂—O—C(O)H |
| 1110 | 2-F,6-CH₃ | C(O)N(CH₃)—(CH₂)₂—N(CH₃)—(CH₂)₂—OH |
| 1111 | 2-F,6-CH₃ | C(O)N(CH₃)—(CH₂)₂—N(CH₃)—(CH₂)₂—NH₂ |
| 1112 | 2-F,6-CH₃ | C(O)N(CH₃)—(CH₂)₂—N(CH₃)—(CH₂)₂—NHCH₃ |
| 1113 | 2-F,6-CH₃ | C(O)N(CH₃)—(CH₂)₂—N(CH₃)—(CH₂)₂—N(CH₃)₂ |
| 1114 | 2-F,6-CH₃ | C(O)N(CH₃)—(CH₂)₂—N(CH₃)—(CH₂)₂—O—C(O)H |
| 1115 | 2-F,6-CH₃ | C(O)O—(CH₂)₂—OH |
| 1116 | 2-F,6-CH₃ | C(O)O—(CH₂)₂—NH₂ |
| 1117 | 2-F,6-CH₃ | C(O)O—(CH₂)₂—NHCH₃ |
| 1118 | 2-F,6-CH₃ | C(O)O—(CH₂)₂—N(CH₃)₂ |
| 1119 | 2-F,6-CH₃ | C(O)O—(CH₂)₂—O—C(O)H |
| 1120 | 2-F,6-CH₃ | C(O)O—(CH₂)₃—OH |
| 1121 | 2-F,6-CH₃ | C(O)O—(CH₂)₃—NH₂ |
| 1122 | 2-F,6-CH₃ | C(O)O—(CH₂)₃—NHCH₃ |
| 1123 | 2-F,6-CH₃ | C(O)O—(CH₂)₃—N(CH₃)₂ |
| 1124 | 2-F,6-CH₃ | C(O)O—(CH₂)₃—O—C(O)H |
| 1125 | 2-F,6-CH₃ | C(O)O—CH(CH₃)—CH₂—OH |
| 1126 | 2-F,6-CH₃ | C(O)O—CH(CH₃)—CH₂—NH₂ |
| 1127 | 2-F,6-CH₃ | C(O)O—CH(CH₃)—CH₂—NHCH₃ |
| 1128 | 2-F,6-CH₃ | C(O)O—CH(CH₃)—CH₂—N(CH₃)₂ |
| 1129 | 2-F,6-CH₃ | C(O)O—CH(CH₃)—CH₂—O—C(O)H |
| 1130 | 2-F,6-CH₃ | C(O)O—CH₂—CH(CH₃)—OH |
| 1131 | 2-F,6-CH₃ | C(O)O—CH₂—CH(CH₃)—NH₂ |
| 1132 | 2-F,6-CH₃ | C(O)O—CH₂—CH(CH₃)—NHCH₃ |
| 1133 | 2-F,6-CH₃ | C(O)O—CH₂—CH(CH₃)—N(CH₃)₂ |
| 1134 | 2-F,6-CH₃ | C(O)O—CH₂—CH(CH₃)—O—C(O)H |
| 1135 | 2-F,6-CH₃ | C(O)O—(CH₂)₂—O—(CH₂)₂—OH |
| 1136 | 2-F,6-CH₃ | C(O)O—(CH₂)₂—O—(CH₂)₂—NH₂ |
| 1137 | 2-F,6-CH₃ | C(O)O—(CH₂)₂—O—(CH₂)₂—NHCH₃ |
| 1138 | 2-F,6-CH₃ | C(O)O—(CH₂)₂—O—(CH₂)₂—N(CH₃)₂ |
| 1139 | 2-F,6-CH₃ | C(O)O—(CH₂)₂—O—(CH₂)₂—O—C(O)H |
| 1140 | 2-F,6-CH₃ | C(O)O—(CH₂)₂—NH—(CH₂)₂—OH |
| 1141 | 2-F,6-CH₃ | C(O)O—(CH₂)₂—NH—(CH₂)₂—NH₂ |
| 1142 | 2-F,6-CH₃ | C(O)O—(CH₂)₂—NH—(CH₂)₂—NHCH₃ |
| 1143 | 2-F,6-CH₃ | C(O)O—(CH₂)₂—NH—(CH₂)₂—N(CH₃)₂ |
| 1144 | 2-F,6-CH₃ | C(O)O—(CH₂)₂—NH—(CH₂)₂—O—C(O)H |
| 1145 | 2-F,6-CH₃ | C(O)O—(CH₂)₂—N(CH₃)—(CH₂)₂—OH |
| 1146 | 2-F,6-CH₃ | C(O)O—(CH₂)₂—N(CH₃)—(CH₂)₂—NH₂ |
| 1147 | 2-F,6-CH₃ | C(O)O—(CH₂)₂—N(CH₃)—(CH₂)₂—NHCH₃ |
| 1148 | 2-F,6-CH₃ | C(O)O—(CH₂)₂—N(CH₃)—(CH₂)₂—N(CH₃)₂ |
| 1149 | 2-F,6-CH₃ | C(O)O—(CH₂)₂—N(CH₃)—(CH₂)₂—O—C(O)H |
| 1150 | 2-F,6-CH₃ | O—(CH₂)₃—OCH₃ |
| 1151 | 2-F,6-CH₃ | O—(CH₂)₃-Piperidin-1-yl |
| 1152 | 2-F,6-CH₃ | O—(CH₂)₃-Piperazin-1-yl |
| 1153 | 2-F,6-CH₃ | O—(CH₂)₂-4-Methylpiperazinyl |
| 1154 | 2-F,6-CH₃ | Morpholin-1-yl |
| 1155 | 2-F,6-CH₃ | O—(CH₂)₃—OH |
| 1156 | 2-F,6-CH₃ | O—(CH₂)₃—NH₂ |
| 1157 | 2-F,6-CH₃ | O—(CH₂)₃—NHCH₃ |
| 1158 | 2-F,6-CH₃ | O—(CH₂)₃—N(CH₃)₂ |
| 1159 | 2-F,6-CH₃ | O—(CH₂)₃—O—C(O)H |
| 1160 | 2-F,6-CH₃ | O—CH(CH₃)—CH₂—OH |
| 1161 | 2-F,6-CH₃ | O—CH(CH₃)—CH₂—NH₂ |
| 1162 | 2-F,6-CH₃ | O—CH(CH₃)—CH₂—NHCH₃ |
| 1163 | 2-F,6-CH₃ | O—CH(CH₃)—CH₂—N(CH₃)₂ |
| 1164 | 2-F,6-CH₃ | O—CH(CH₃)—CH₂—O—C(O)H |
| 1165 | 2-F,6-CH₃ | O—CH₂—CH(CH₃)—OH |
| 1166 | 2-F,6-CH₃ | O—CH₂—CH(CH₃)—NH₂ |
| 1167 | 2-F,6-CH₃ | O—CH₂—CH(CH₃)—NHCH₃ |
| 1168 | 2-F,6-CH₃ | O—CH₂—CH(CH₃)—N(CH₃)₂ |
| 1169 | 2-F,6-CH₃ | O—CH₂—CH(CH₃)—O—C(O)H |
| 1170 | 2-F,6-CH₃ | O—(CH₂)₂—O—(CH₂)₂—OH |
| 1171 | 2-F,6-CH₃ | O—(CH₂)₂—O—(CH₂)₂—NH₂ |
| 1172 | 2-F,6-CH₃ | O—(CH₂)₂—O—(CH₂)₂—NHCH₃ |
| 1173 | 2-F,6-CH₃ | O—(CH₂)₂—O—(CH₂)₂—N(CH₃)₂ |
| 1174 | 2-F,6-CH₃ | O—(CH₂)₂—O—(CH₂)₂—O—C(O)H |
| 1175 | 2-F,6-CH₃ | O—(CH₂)₂—NH—(CH₂)₂—OH |
| 1176 | 2-F,6-CH₃ | O—(CH₂)₂—NH—(CH₂)₂—NH₂ |
| 1177 | 2-F,6-CH₃ | O—(CH₂)₂—NH—(CH₂)₂—NHCH₃ |
| 1178 | 2-F,6-CH₃ | O—(CH₂)₂—NH—(CH₂)₂—N(CH₃)₂ |
| 1179 | 2-F,6-CH₃ | O—(CH₂)₂—NH—(CH₂)₂—O—C(O)H |
| 1180 | 2-F,6-CH₃ | O—(CH₂)₂—N(CH₃)—(CH₂)₂—OH |
| 1181 | 2-F,6-CH₃ | O—(CH₂)₂—N(CH₃)—(CH₂)₂—NH₂ |
| 1182 | 2-F,6-CH₃ | O—(CH₂)₂—N(CH₃)—(CH₂)₂—NHCH₃ |
| 1183 | 2-F,6-CH₃ | O—(CH₂)₂—N(CH₃)—(CH₂)₂—N(CH₃)₂ |
| 1184 | 2-F,6-CH₃ | O—(CH₂)₂—N(CH₃)—(CH₂)₂—O—C(O)H |
| 1185 | 2-F,6-CH₃ | NH—(CH₂)₂—OH |
| 1186 | 2-F,6-CH₃ | NH—(CH₂)₂—NH₂ |
| 1187 | 2-F,6-CH₃ | NH—(CH₂)₂—NHCH₃ |
| 1188 | 2-F,6-CH₃ | NH—(CH₂)₂—N(CH₃)₂ |
| 1189 | 2-F,6-CH₃ | NH—(CH₂)₂—O—C(O)H |
| 1190 | 2-F,6-CH₃ | NH—(CH₂)₃—OH |
| 1191 | 2-F,6-CH₃ | NH—(CH₂)₃—NH₂ |
| 1192 | 2-F,6-CH₃ | NH—(CH₂)₃—NHCH₃ |
| 1193 | 2-F,6-CH₃ | NH—(CH₂)₃—N(CH₃)₂ |
| 1194 | 2-F,6-CH₃ | NH—(CH₂)₃—O—C(O)H |
| 1195 | 2-F,6-CH₃ | NH—CH(CH₃)—CH₂—OH |
| 1196 | 2-F,6-CH₃ | NH—CH(CH₃)—CH₂—NH₂ |
| 1197 | 2-F,6-CH₃ | NH—CH(CH₃)—CH₂—NHCH₃ |
| 1198 | 2-F,6-CH₃ | NH—CH(CH₃)—CH₂—N(CH₃)₂ |
| 1199 | 2-F,6-CH₃ | NH—CH(CH₃)—CH₂—O—C(O)H |
| 1200 | 2-F,6-CH₃ | NH—CH₂—CH(CH₃)—OH |
| 1201 | 2-F,6-CH₃ | NH—CH₂—CH(CH₃)—NH₂ |
| 1202 | 2-F,6-CH₃ | NH—CH₂—CH(CH₃)—NHCH₃ |
| 1203 | 2-F,6-CH₃ | NH—CH₂—CH(CH₃)—N(CH₃)₂ |
| 1204 | 2-F,6-CH₃ | NH—CH₂—CH(CH₃)—O—C(O)H |
| 1205 | 2-F,6-CH₃ | NH—(CH₂)₂—O—(CH₂)₂—OH |
| 1206 | 2-F,6-CH₃ | NH—(CH₂)₂—O—(CH₂)₂—NH₂ |
| 1207 | 2-F,6-CH₃ | NH—(CH₂)₂—O—(CH₂)₂—NHCH₃ |
| 1208 | 2-F,6-CH₃ | NH—(CH₂)₂—O—(CH₂)₂—N(CH₃)₂ |
| 1209 | 2-F,6-CH₃ | NH—(CH₂)₂—O—(CH₂)₂—O—C(O)H |
| 1210 | 2-F,6-CH₃ | NH—(CH₂)₂—NH—(CH₂)₂—OH |
| 1211 | 2-F,6-CH₃ | NH—(CH₂)₂—NH—(CH₂)₂—NH₂ |
| 1212 | 2-F,6-CH₃ | NH—(CH₂)₂—NH—(CH₂)₂—NHCH₃ |
| 1213 | 2-F,6-CH₃ | NH—(CH₂)₂—NH—(CH₂)₂—N(CH₃)₂ |
| 1214 | 2-F,6-CH₃ | NH—(CH₂)₂—NH—(CH₂)₂—O—C(O)H |
| 1215 | 2-F,6-CH₃ | NH—(CH₂)₂—N(CH₃)—(CH₂)₂—OH |
| 1216 | 2-F,6-CH₃ | NH—(CH₂)₂—N(CH₃)—(CH₂)₂—NH₂ |
| 1217 | 2-F,6-CH₃ | NH—(CH₂)₂—N(CH₃)—(CH₂)₂—NHCH₃ |
| 1218 | 2-F,6-CH₃ | NH—(CH₂)₂—N(CH₃)—(CH₂)₂—N(CH₃)₂ |
| 1219 | 2-F,6-CH₃ | NH—(CH₂)₂—N(CH₃)—(CH₂)₂—O—C(O)H |
| 1220 | 2-F,6-CH₃ | N(CH₃)—(CH₂)₂—OH |
| 1221 | 2-F,6-CH₃ | N(CH₃)—(CH₂)₂—NH₂ |
| 1222 | 2-F,6-CH₃ | N(CH₃)—(CH₂)₂—NHCH₃ |
| 1223 | 2-F,6-CH₃ | N(CH₃)—(CH₂)₂—N(CH₃)₂ |
| 1224 | 2-F,6-CH₃ | N(CH₃)—(CH₂)₂—O—C(O)H |
| 1225 | 2-F,6-CH₃ | N(CH₃)—(CH₂)₃—OH |
| 1226 | 2-F,6-CH₃ | N(CH₃)—(CH₂)₃—NH₂ |
| 1227 | 2-F,6-CH₃ | N(CH₃)—(CH₂)₃—NHCH₃ |
| 1228 | 2-F,6-CH₃ | N(CH₃)—(CH₂)₃—N(CH₃)₂ |
| 1229 | 2-F,6-CH₃ | N(CH₃)—(CH₂)₃—O—C(O)H |
| 1230 | 2-F,6-CH₃ | N(CH₃)—CH(CH₃)—CH₂—OH |
| 1231 | 2-F,6-CH₃ | N(CH₃)—CH(CH₃)—CH₂—NH₂ |
| 1232 | 2-F,6-CH₃ | N(CH₃)—CH(CH₃)—CH₂—NHCH₃ |
| 1233 | 2-F,6-CH₃ | N(CH₃)—CH(CH₃)—CH₂—N(CH₃)₂ |
| 1234 | 2-F,6-CH₃ | N(CH₃)—CH(CH₃)—CH₂—O—C(O)H |
| 1235 | 2-F,6-CH₃ | N(CH₃)—CH₂—CH(CH₃)—OH |
| 1236 | 2-F,6-CH₃ | N(CH₃)—CH₂—CH(CH₃)—NH₂ |
| 1237 | 2-F,6-CH₃ | N(CH₃)—CH₂—CH(CH₃)—NHCH₃ |
| 1238 | 2-F,6-CH₃ | N(CH₃)—CH₂—CH(CH₃)—N(CH₃)₂ |
| 1239 | 2-F,6-CH₃ | N(CH₃)—CH₂—CH(CH₃)—O—C(O)H |
| 1240 | 2-F,6-CH₃ | N(CH₃)—(CH₂)₂—O—(CH₂)₂—OH |
| 1241 | 2-F,6-CH₃ | N(CH₃)—(CH₂)₂—O—(CH₂)₂—NH₂ |
| 1242 | 2-F,6-CH₃ | N(CH₃)—(CH₂)₂—O—(CH₂)₂—NHCH₃ |
| 1243 | 2-F,6-CH₃ | N(CH₃)—(CH₂)₂—O—(CH₂)₂—N(CH₃)₂ |
| 1244 | 2-F,6-CH₃ | N(CH₃)—(CH₂)₂—O—(CH₂)₂—O—C(O)H |
| 1245 | 2-F,6-CH₃ | N(CH₃)—(CH₂)₂—NH—(CH₂)₂—OH |
| 1246 | 2-F,6-CH₃ | N(CH₃)—(CH₂)₂—NH—(CH₂)₂—NH₂ |
| 1247 | 2-F,6-CH₃ | N(CH₃)—(CH₂)₂—NH—(CH₂)₂—NHCH₃ |
| 1248 | 2-F,6-CH₃ | N(CH₃)—(CH₂)₂—NH—(CH₂)₂—N(CH₃)₂ |
| 1249 | 2-F,6-CH₃ | N(CH₃)—(CH₂)₂—NH—(CH₂)₂—O—C(O)H |
| 1250 | 2-F,6-CH₃ | N(CH₃)—(CH₂)₂—N(CH₃)—(CH₂)₂—OH |
| 1251 | 2-F,6-CH₃ | N(CH₃)—(CH₂)₂—N(CH₃)—(CH₂)₂—NH₂ |
| 1252 | 2-F,6-CH₃ | N(CH₃)—(CH₂)₂—N(CH₃)—(CH₂)₂—NHCH₃ |
| 1253 | 2-F,6-CH₃ | N(CH₃)—(CH₂)₂—N(CH₃)—(CH₂)₂—N(CH₃)₂ |
| 1254 | 2-F,6-CH₃ | N(CH₃)—(CH₂)₂—N(CH₃)—(CH₂)₂—O—C(O)H |

TABLE A

| No. | |
|---|---|
| A-1 | Cyclopropyl |
| A-2 | Cyclobutyl |
| A-3 | Cyclopentyl |
| A-4 | Cyclohexyl |
| A-5 | N(H)—CH₂—CF₃ |
| A-6 | N(H)—CH(CH₃)—CF₃ (rac.) |
| A-7 | N(H)—CH(CH₃)—CF₃ (R) |
| A-8 | N(H)—CH(CH₃)—CF₃ (S) |
| A-9 | N(H)—CH₂—CH₂—CF₃ |
| A-10 | N(H)—CH₂—CH₂—CF═CF₂ |
| A-11 | N(CH₃)—CH₂—CF₃ |
| A-12 | N(CH₃)—CH(CH₃)—CF₃ (rac.) |
| A-13 | N(CH₃)—CH(CH₃)—CF₃ (R) |
| A-14 | N(CH₃)—CH(CH₃)—CF₃ (S) |
| A-15 | N(CH₃)—CH₂—CH₂—CF₃ |
| A-16 | N(CH₃)—CH₂—CH₂—CF═CF₂ |
| A-17 | N(C₂H₅)—CH₂—CF₃ |
| A-18 | N(C₂H₅)—CH(CH₃)—CF₃ (rac.) |
| A-19 | N(C₂H₅)—CH(CH₃)—CF₃ (R) |
| A-20 | N(C₂H₅)—CH(CH₃)—CF₃ (S) |
| A-21 | N(C₂H₅)—CH₂—CH₂—CF₃ |
| A-22 | N(C₂H₅)—CH₂—CH₂—CF═CF₂ |
| A-23 | 2,2-Dichlorocyclopropyl |
| A-24 | 2,2-Dichloro-3-methylcyclopropyl |
| A-25 | 2,2-Dichloro-1-methylcyclopropyl |

In addition to the compounds individualized in Tables 1 to 1254, the corresponding derivatives in which X is cyano also constitute further subjects of the invention.

In addition to the compounds individualized in Tables 1 to 1254, the corresponding derivatives in which X is methyl also constitute further subjects of the invention.

In addition to the compounds individualized in Tables 1 to 1254, the corresponding derivatives in which X is methoxy also constitute further subjects of the invention.

In addition to the compounds individualized in Tables 1 to 1254, the corresponding derivatives in which X is fluorine also constitute further subjects of the invention.

Some of the compounds of the formula I are known and some of them are novel.

The invention therefore also provides azolopyrimidines of the formula I in which

### Y¹ is C(O)O, C(O)NR^{A}, or S(O)ₜ,

t is 1 or 2 and
the remaining symbols and indices each have the definitions and preferences specified for the formula I.

The invention further provides azolopyrimidines of the formula I in which

T is OR^{c} (where R^{c}≠H), OC(O)R^{a}, NR^{b}R^{b′}, C(O)NR^{b}R^{b}, C(NOR^{c})R^{a},
T¹-C(=T²)-T³, and, if R is defined as NR¹R² or C₃-C₁₂-halocycloalkyl, is also C(O)ORC or C(OR^{c})₂R^{a},
   and the remaining symbols and indices each have the definitions and preferences specified for the formula I.

The invention further provides azolopyrimidines of the formula I in which T is substituted by at least one oxo group (═O).

The invention further provides compounds of the formula I in which

W is five- or six-membered heteroaryl which, as well as carbon atoms, comprises one, two or three further heteroatoms from the group of O, N and S as ring members, where the ring systems, as well as Lₘ groups, bear at least one substituent P₁,
and the remaining symbols and indices each have the definitions and preferences specified for the formula I.

The invention further provides azolopyrimidines of the formula I in which the symbols and indices are as defined as follows:

### G, E, Q a)G is N; E is C—W² and Q is C—W³;

b) G is C—W¹; E is C—W² and Q is N; or
c) G is C—W¹; E is N and Q is C—W³;
   and the remaining symbols and indices each have the definitions and preferences specified for the formula I.

The invention further provides compounds of the formula I where

X is F, I, cyano, C₁-C₄-alkyl, C₁-C₄-haloalkyl, C₁-C₄-alkoxy or C₁-C₄-haloalkoxy and
the remaining symbols and indices each have the definitions and preferences specified for the formula I.

The invention further provides compounds of the formula I where

R is C₃-C₁₂-halocycloalkyl and
the remaining symbols and indices each have the definitions and preferences specified for the formula I.

The invention further provides compounds of the formula I where

T is OC(O)R^{a} and the remaining symbols and indices each have the definitions and preferences specified for the formula I.

The compounds I are suitable for use as fungicides. They have excellent activity against a broad spectrum of phytopathogenic fungi from the class of the Ascomycetes, Deuteromycetes, Basidiomycetes and Peronosporomycetes (syn. Oomycetes). Some of them are systemically active and can be used in crop protection as foliar fungicides, as fungicides for seed dressing and as soil fungicides.

They are particularly important for the control of a large number of fungi on various crop plants, such as wheat, rye, barley, oats, rice, corn, grass, bananas, cotton, soybeans, coffee, sugarcane, grapevines, fruit and ornamental plants and vegetables, such as cucumbers, beans, tomatoes, potatoes and cucurbits, and also on the seeds of these plants.

The compounds I are suitable for controlling Alternaria species on vegetables, rapeseed, sugarbeet and fruit and rice, for example A. solani or A. alternata on potatoes and tomatoes.

The compounds I are suitable for controlling Aphanomyces species on sugarbeet and vegetables.

The compounds I are suitable for controlling Ascochyta species on cereals and vegetables.

The compounds I are suitable for controlling Bipolaris and Drechslera species on corn, cereals, rice and lawns, for example D. maydis on corn.

The compounds I are suitable for controlling Blumeria graminis (powdery mildew) on cereals.

The compounds I are suitable for controlling Botrytis cinerea (gray mold) on strawberries, vegetables, flowers and grapevines.

The compounds I are suitable for controlling Bremia lactucae on lettuce.

The compounds I are suitable for controlling Cercospora species on corn, soybeans, rice and sugarbeet.

The compounds I are suitable for controlling Cochliobolus species on corn, cereals, rice, for example Cochliobolus sativus on cereals, Cochliobolus miyabeanus on rice.

The compounds I are suitable for controlling Colletotricum species on soybeans and cotton.

The compounds I are suitable for controlling Drechslera species, Pyrenophora species on corn, cereals, rice and lawns, for example D. teres on barley or D. tritici-repentis on wheat.

The compounds I are suitable for controlling Esca on grapevines, caused by Phaeoacremonium chlamydosporium, Ph. Aleophilum, and Formitipora punctata (syn. Phellinus punctatus).

The compounds I are suitable for controlling Exserohilum species on corn.

The compounds I are suitable for controlling Erysiphe cichoracearum and Sphaerotheca fuliginea on cucumbers.

The compounds I are suitable for controlling Fusarium and Verticillium species on various plants, for example F. graminearum or F. culmorum on cereals or F. oxysporum on a large number of plants, for example tomatoes.

The compounds I are suitable for controlling Gaeumanomyces graminis on cereals.

The compounds I are suitable for controlling Gibberella species on cereals and rice (for example Gibberella fujikuroi on rice).

The compounds I are suitable for controlling Grainstaining complex on rice.

The compounds I are suitable for controlling Helminthosporium species on corn and rice.

The compounds I are suitable for controlling Michrodochium nivale on cereals.

The compounds I are suitable for controlling Mycosphaerella species on cereals, bananas and peanuts, for example M. graminicola on wheat or M. fijiensis on bananas.

The compounds I are suitable for controlling Peronospora species on cabbage and bulbous plants, for example P. brassicae on cabbage or P. destructoron onions.

The compounds I are suitable for controlling Phakopsara pachyrhizi and Phakopsara meibomiae on soybeans.

The compounds I are suitable for controlling Phomopsis species on soybeans and sunflowers.

The compounds I are suitable for controlling Phytophthora infestans on potatoes and tomatoes.

The compounds I are suitable for controlling Phytophthora species on various plants, for example P. capsici on bell peppers.

The compounds I are suitable for controlling Plasmopara viticola on grapevines.

The compounds I are suitable for controlling Podosphaera leucotricha on apples.

The compounds I are suitable for controlling Pseudocercosporella herpotrichoides on cereals.

The compounds I are suitable for controlling Pseudoperonospora on various plants, for example P. cubensis on cucumbers or P. humili on hops.

The compounds I are suitable for controlling Puccinia species on various plants, for example P. triticina, P. striformins, P. hordei or P. graminis on cereals, or P. asparagi on asparagus.

The compounds I are suitable for controlling Pyricularia oryzae, Corticium sasakii, Sarocladium oryzae, S. attenuatum, Entyloma oryzae on rice.

The compounds I are suitable for controlling Pyricularia grisea on lawns and cereals.

The compounds I are suitable for controlling Pythium spp. on lawns, rice, corn, cotton, rapeseed, sunflowers, sugarbeet, vegetables and other plants, for example P. ultiumum on various plants, P. aphanidermatum on lawns.

The compounds I are suitable for controlling Rhizoctonia species on cotton, rice, potatoes, lawns, corn, rapeseed, potatoes, sugarbeet, vegetables and on various plants, for example R. solani on beet and various plants.

The compounds I are suitable for controlling Rhynchosporium secalis on barley, rye and triticale.

The compounds I are suitable for controlling Scierotinia species on rapeseed and sunflowers.

The compounds I are suitable for controlling Septoria tritici and Stagonospora nodorum on wheat.

The compounds I are suitable for controlling Erysiphe (syn. Uncinula) necator on grapevines.

The compounds I are suitable for controlling Setospaeria species on corn and lawns.

The compounds I are suitable for controlling Sphacelotheca reilinia on corn.

The compounds I are suitable for controlling Thievaliopsis species on soybeans and cotton.

The compounds I are suitable for controlling Tilletia species on cereals.

The compounds I are suitable for controlling Ustilago species on cereals, corn and sugarcane, for example U. maydis on corn.

The compounds I are suitable for controlling Venturia species (scab) on apples and pears, for example V. inaequalis on apples.

In addition, the inventive compounds can also be used in crops which, owing to breeding including genetic engineering, are tolerant to attack by insects or fungi.

The compounds I are also suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The inventive compounds and/or their agriculturally acceptable salts are employed by treating the fungi or the plants, seeds or materials to be protected against fungal attack or the soil with a fungicidally effective amount of the active ingredients. Application can be both before and after the infection of the materials, plants or seeds by the fungi.

The invention therefore further provides a method for controlling phytopathogenic fungi wherein the fungi or the materials, plants, the soil or seeds to be protected against fungal attack are/is treated with an effective amount of at least one compound I according to the invention and/or an agriculturally acceptable salt thereof.

The invention further provides a composition for controlling phytopathogenic fungi, which composition comprises at least one inventive compound of the formula (Ia) and/or an agriculturally acceptable salt thereof and at least one solid or liquid carrier.

The fungicidal compositions generally comprise between 0.1 and 95% by weight, preferably between 0.5 and 90% by weight, of active ingredient.

When employed in crop protection, the application rates are, depending on the kind of effect desired, between 0.01 and 2.0 kg of active ingredient per ha.

In seed treatment, the amounts of active ingredient required are generally from 1 to 1000 g/100 kg of seed, preferably from 5 to 100 g/100 kg of seed.

When used in the protection of materials or stored products, the active ingredient application rate depends on the kind of application area and on the desired effect. Amounts typically applied in the protection of materials are, for example, from 0.001 g to 2 kg, preferably from 0.005 g to 1 kg, of active ingredient per cubic meter of treated material.

The compounds of the formula I can be present in different crystal modifications which may differ in their biological activity. They likewise form part of the subject matter of the present invention.

The compounds I can be converted to the customary formulations, for example solutions, emulsions, suspensions, dusts, powders, pastes and granules. The application form depends on the particular purpose; in each case, it should ensure a fine and uniform distribution of the inventive compound.

The formulations are prepared in a known manner, for example by extending the active ingredient with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used
carriers such as ground natural minerals (for example kaolins, clays, talc, chalk) and ground synthetic minerals (for example finely divided silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable for use as surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, and also condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, and also coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or cogrinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, for example ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active ingredient. The active ingredients are employed in a purity of from 90% to 100%, preferably 95% to 100% (according to NMR spectrum).

The following are examples of formulations: 1. Products for dilution with water

A Water-Soluble Concentrates (SL, LS)

10 parts by weight of the active ingredients are dissolved with 90 parts by weight of water or with a water-soluble solvent. As an alternative, wetters or other auxiliaries are added. The active ingredient dissolves upon dilution with water. This gives a formulation having an active ingredient content of 10% by weight.

B Dispersible Concentrates (DC)

20 parts by weight of the active ingredients are dissolved in 70 parts by weight of cyclohexanone with addition of 10 parts by weight of a dispersant, for example polyvinylpyrrolidone. Dilution with water gives a dispersion. The active ingredient content is 20% by weight

C Emulsifiable Concentrates (EC)

15 parts by weight of the active ingredients are dissolved in 75 parts by weight of xylene with addition of calcium dodecylbenzenesulfonate and castor oil ethoxylate (in each case 5 parts by weight). Dilution with water gives an emulsion. The formulation has an active ingredient content of 15% by weight.

D Emulsions (EW, EO, ES)

25 parts by weight of the active ingredients are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The formulation has an active ingredient content of 25% by weight.

E Suspensions (SC, QD, FS)

In an agitated ball mill, 20 parts by weight of the active ingredients are comminuted with addition of 10 parts by weight of dispersants and wetters and 70 parts by weight of water or an organic solvent to give a fine active ingredient suspension. Dilution with water gives a stable suspension of the active ingredient. The active ingredient content in the formulation is 20% by weight.

F Water-Dispersible Granules and Water-Soluble Granules (WG, SG)

50 parts by weight of the active ingredients are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active ingredient. The formulation has an active ingredient content of 50% by weight.

G Water-Dispersible Powders and Water-Soluble Powders (WP, SP, SS, WS)

75 parts by weight of the active ingredients are ground in a rotor-stator mill with addition of 25 parts by weight of dispersants, wetters and silica gel. Dilution with water gives a stable dispersion or solution of the active ingredient. The active ingredient content of the formulation is 75% by weight.

H Gel Formulations (GF)

20 parts by weight of the active ingredients, 10 parts by weight of dispersant, 1 part by weight of gelling agent and 70 parts by weight of water or an organic solvent are ground in a ball mill to give a fine suspension. Dilution with water gives a stable suspension with an active ingredient content of 20% by weight.

2. Products to be Applied Undiluted

I Dusts (DP, DS)

5 parts by weight of the active ingredients are ground finely and mixed intimately with 95 parts by weight of finely divided kaolin. This gives a dustable product with an active ingredient content of 5% by weight.

J Granules (GR, FG, GG, MG)

0.5 part by weight of the active ingredients is ground finely and associated with 99.5 parts by weight of carriers. Current methods are extrusion, spray-drying or the fluidized bed. This gives granules with an active ingredient content of 0.5% by weight to be applied undiluted.

K ULV Solutions (UL)

10 parts by weight of the active ingredients are dissolved in 90 parts by weight of an organic solvent, for example xylene. This gives a product with an active ingredient content of 10% by weight to be applied undiluted.

Water-soluble concentrates (LS), suspensions (FS), dusts (DS), water-dispersible and water-soluble powders (WS, SS), emulsions (ES), emulsifiable concentrates (EC) and gel formulations (GF) are usually used for the treatment of seed. These formulations can be applied to the seed in undiluted or, preferably, diluted form. The application can be carried out before sowing.

The active ingredients can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the inventive active ingredients.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use preparations can be varied within relatively wide ranges. In general, they are from 0.0001 to 10%, preferably from 0.01 to 1%.

The active ingredients may also be used successfully in the ultra-low-volume process (ULV), by which it is possible to apply formulations comprising over 95% by weight of active ingredient, or even to apply the active ingredient without additives.

Various types of oils, wetters, adjuvants, herbicides, fungicides, other pesticides, or bactericides may be added to the active ingredients, if appropriate not until immediately prior to use (tank mix). These compositions can be admixed with the compositions according to the invention in a weight ratio of from 1:100 to 100:1, preferably from 1:10 to 10:1.

The following are particularly suitable as adjuvants in this context: organically modified polysiloxanes, for example Break Thru S 240®; alcohol alkoxylates, for example Atplus 245®, Atplus MBA 1303®, Plurafac LF 300® and Lutensol ON 30®; EO-PO block polymers, for example Pluronic RPE 2035® and Genapol B®; alcohol ethoxylates, for example Lutensol XP 80®; and sodium dioctylsulfosuccinate, for example Leophen RA®.

The inventive compounds in the application form as fungicides can also be present together with other active ingredients, for example with herbicides, insecticides, growth regulators, fungicides or else with fertilizers. When mixing the inventive compounds or the compositions comprising them with one or more further active ingredients, in particular fungicides, it is in many cases possible, for example, to widen the activity spectrum or to prevent the development of resistance. In many cases, synergistic effects are obtained.

The invention thereof further provides a combination of at least one inventive compound of the formula (I) and/or an agriculturally acceptable salt thereof and at least one further fungicidal, insecticidal, herbicidal and/or growth-regulating active ingredient.

The following list of fungicides with which the inventive compounds can be applied together is intended to illustrate the possible combinations, but not to limit them:

strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethylphenyloxymethylene)phenyl)-3-methoxyacrylate; carboxamides
carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(3′,4′-dichloro-5-fluorobiphenyl-2-yl)-3-difluoromethyl-1-methylpyrazole-4-carboxamide, N-(2-cyanophenyl)-3,4-dichloroisothiazole-5-carboxamide;
carboxylic morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)ethyl)-2-ethanesulfonylamino-3-methylbutyramide;
   azoles
triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;
   heterocyclic nitrogen compounds
pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-5-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazole-1-sulfonamide;
   carbamates and dithiocarbamates
dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate;
   other fungicides
guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active ingredients: Bordeaux mixture, copper acetate, copper hydroxide, copper oxychloride, basic copper sulfate, sulfur;
others: spiroxamine, cyflufenamid, cymoxanil, metrafenone.

Accordingly, the present invention further relates to the compositions listed in Table B, where one row of Table B corresponds in each case to a fungicidal composition comprising a compound of the formula I (component 1), which is preferably one of the compounds described herein as being preferred, and the particular further active ingredient (component 2) stated in the row in question. According to one embodiment of the invention, component 1 in each row of Table B is in each case one of the compounds of the formula I specifically individualized in Tables 1 to 1254.

TABLE B

| Row | Component 1 | Component 2 |
|---|---|---|
| B-1 | a compound of the formula I | azoxystrobin |
| B-2 | a compound of the formula I | dimoxystrobin |
| B-3 | a compound of the formula I | enestroburin |
| B-4 | a compound of the formula I | fluoxastrobin |
| B-5 | a compound of the formula I | kresoxim-methyl |
| B-6 | a compound of the formula I | metominostrobin |
| B-7 | a compound of the formula I | picoxystrobin |
| B-8 | a compound of the formula I | pyraclostrobin |
| B-9 | a compound of the formula I | trifloxystrobin |
| B-10 | a compound of the formula I | orysastrobin |
| B-11 | a compound of the formula I | methyl (2-chloro-5-[1-(3-methylbenzyloxy- |
| | | imino)ethyl]benzyl)carbamate |
| B-12 | a compound of the formula I | methyl (2-chloro-5-[1-(6-methylpyridin-2-yl- |
| | | methoxyimino)ethyl]benzyl)carbamate |
| B-13 | a compound of the formula I | methyl 2-(ortho-(2,5-dimethylphenyloxy- |
| | | methylene)phenyl)-3-methoxyacrylate |
| B-14 | a compound of the formula I | benalaxyl |
| B-15 | a compound of the formula I | benodanil |
| B-16 | a compound of the formula I | boscalid |
| B-17 | a compound of the formula I | carboxin |
| B-18 | a compound of the formula I | mepronil |
| B-19 | a compound of the formula I | fenfuram |
| B-20 | a compound of the formula I | fenhexamid |
| B-21 | a compound of the formula I | flutolanil |
| B-22 | a compound of the formula I | furametpyr |
| B-23 | a compound of the formula I | metalaxyl |
| B-24 | a compound of the formula I | ofurace |
| B-25 | a compound of the formula I | oxadixyl |
| B-26 | a compound of the formula I | oxycarboxin |
| B-27 | a compound of the formula I | penthiopyrad |
| B-28 | a compound of the formula I | thifluzamide |
| B-29 | a compound of the formula I | tiadinil |
| B-30 | a compound of the formula I | N-(4′-bromobiphenyl-2-yl)-4-difluoromethyl- |
| | | 2-methylthiazole-5-carboxamide |
| B-31 | a compound of the formula I | N-(4′-trifluoromethylbiphenyl-2-yl)-4-di- |
| | | fluoromethyl-2-methylthiazole-5-carboxamide |
| B-32 | a compound of the formula I | N-(4′-chloro-3′-fluorobiphenyl-2-yl)-4-di- |
| | | fluoromethyl-2-methylthiazole-5-carboxamide |
| B-33 | a compound of the formula I | N-(3′,4′-dichloro-4-fluorobiphenyl-2-yl)-3-di- |
| | | fluoromethyl-1-methylpyrazole-4-carboxamide |
| B-34 | a compound of the formula I | N-(3′,4′-dichloro-5-fluorobiphenyl-2-yl)-3-di- |
| | | fluoromethyl-1-methylpyrazole-4-carboxamide |
| B-35 | a compound of the formula I | N-(2-cyanophenyl)-3,4-dichloroisothiazole- |
| | | 5-carboxamide |
| B-36 | a compound of the formula I | dimethomorph |
| B-37 | a compound of the formula I | flumorph |
| B-38 | a compound of the formula I | flumetover |
| B-39 | a compound of the formula I | fluopicolide (picobenzamid) |
| B-40 | a compound of the formula I | zoxamide |
| B-41 | a compound of the formula I | carpropamid |
| B-42 | a compound of the formula I | diclocymet |
| B-43 | a compound of the formula I | mandipropamid |
| B-44 | a compound of the formula I | N-(2-(4-[3-(4-chlorophenyl)prop-2-ynyloxy]- |
| | | 3-methoxyphenyl)ethyl)-2-methanesulfonyl- |
| | | amino-3-methylbutyramide |
| B-45 | a compound of the formula I | N-(2-(4-[3-(4-chlorophenyl)prop-2-ynyloxy]- |
| | | 3-methoxyphenyl)ethyl)-2-ethanesulfonyl- |
| | | amino-3-methylbutyramide |
| B-46 | a compound of the formula I | bitertanol |
| B-47 | a compound of the formula I | bromuconazole |
| B-48 | a compound of the formula I | cyproconazole |
| B-49 | a compound of the formula I | difenoconazole |
| B-50 | a compound of the formula I | diniconazole |
| B-51 | a compound of the formula I | enilconazole |
| B-52 | a compound of the formula I | epoxiconazole |
| B-53 | a compound of the formula I | fenbuconazole |
| B-54 | a compound of the formula I | flusilazole |
| B-55 | a compound of the formula I | fluquinconazole |
| B-56 | a compound of the formula I | flutriafol |
| B-57 | a compound of the formula I | hexaconazole |
| B-58 | a compound of the formula I | imibenconazole |
| B-59 | a compound of the formula I | ipconazole |
| B-60 | a compound of the formula I | metconazole |
| B-61 | a compound of the formula I | myclobutanil |
| B-62 | a compound of the formula I | penconazole |
| B-63 | a compound of the formula I | propiconazole |
| B-64 | a compound of the formula I | prothioconazole |
| B-65 | a compound of the formula I | simeconazole |
| B-66 | a compound of the formula I | tebuconazole |
| B-67 | a compound of the formula I | tetraconazole |
| B-68 | a compound of the formula I | triadimenol |
| B-69 | a compound of the formula I | triadimefon |
| B-70 | a compound of the formula I | triticonazole |
| B-71 | a compound of the formula I | cyazofamid |
| B-72 | a compound of the formula I | imazalil |
| B-73 | a compound of the formula I | pefurazoate |
| B-74 | a compound of the formula I | prochloraz |
| B-75 | a compound of the formula I | triflumizole |
| B-76 | a compound of the formula I | benomyl |
| B-77 | a compound of the formula I | carbendazim |
| B-78 | a compound of the formula I | fuberidazole |
| B-79 | a compound of the formula I | thiabendazole |
| B-80 | a compound of the formula I | ethaboxam |
| B-81 | a compound of the formula I | etridiazole |
| B-82 | a compound of the formula I | hymexazole |
| B-83 | a compound of the formula I | fluazinam |
| B-84 | a compound of the formula I | pyrifenox |
| B-85 | a compound of the formula I | 3-[5-(4-chlorophenyl)-2,3-dimethyl- |
| | | isoxazolidin-3-yl]pyridine |
| B-86 | a compound of the formula I | bupirimate |
| B-87 | a compound of the formula I | cyprodinil |
| B-88 | a compound of the formula I | ferimzone |
| B-89 | a compound of the formula I | fenarimol |
| B-90 | a compound of the formula I | mepanipyrim |
| B-91 | a compound of the formula I | nuarimol |
| B-92 | a compound of the formula I | pyrimethanil |
| B-93 | a compound of the formula I | triforine |
| B-94 | a compound of the formula I | fludioxonil |
| B-95 | a compound of the formula I | fenpiclonil |
| B-96 | a compound of the formula I | aldimorph |
| B-97 | a compound of the formula I | dodemorph |
| B-98 | a compound of the formula I | fenpropimorph |
| B-99 | a compound of the formula I | tridemorph |
| B-100 | a compound of the formula I | iprodione |
| B-101 | a compound of the formula I | procymidone |
| B-102 | a compound of the formula I | vinclozolin |
| B-103 | a compound of the formula I | acibenzolar-S-methyl |
| B-104 | a compound of the formula I | anilazin |
| B-105 | a compound of the formula I | captan |
| B-106 | a compound of the formula I | captafol |
| B-107 | a compound of the formula I | dazomet |
| B-108 | a compound of the formula I | diclomezine |
| B-109 | a compound of the formula I | fenoxanil |
| B-110 | a compound of the formula I | folpet |
| B-111 | a compound of the formula I | fenpropidin |
| B-112 | a compound of the formula I | famoxadone |
| B-113 | a compound of the formula I | fenamidone |
| B-114 | a compound of the formula I | octhilinone |
| B-115 | a compound of the formula I | probenazole |
| B-116 | a compound of the formula I | proquinazid |
| B-117 | a compound of the formula I | pyroquilon |
| B-118 | a compound of the formula I | quinoxyfen |
| B-119 | a compound of the formula I | tricyclazole |
| B-120 | a compound of the formula I | 5-chloro-7-(4-methylpiperidin-1-yl)-6-(2,4,6- |
| | | trifluorophenyl)-[1,2,4]triazolo[1,5-a]pyrimidine |
| B-121 | a compound of the formula I | 2-butoxy-6-iodo-3-propylchromen-4-one |
| B-122 | a compound of the formula I | N,N-dimethyl-3-(3-bromo-6-fluoro-2-methyl- |
| | | indole-1-sulfonyl)-[1,2,4]triazole-1-sulfon- |
| | | amide |
| B-123 | a compound of the formula I | ferbam |
| B-124 | a compound of the formula I | mancozeb |
| B-125 | a compound of the formula I | maneb |
| B-126 | a compound of the formula I | metiram |
| B-127 | a compound of the formula I | metam |
| B-128 | a compound of the formula I | propineb |
| B-129 | a compound of the formula I | thiram |
| B-130 | a compound of the formula I | zineb |
| B-131 | a compound of the formula I | ziram |
| B-132 | a compound of the formula I | diethofencarb |
| B-133 | a compound of the formula I | flubenthiavalicarb |
| B-134 | a compound of the formula I | iprovalicarb |
| B-135 | a compound of the formula I | propamocarb |
| B-136 | a compound of the formula I | methyl 3-(4-chlorophenyl)-3-(2-isopropoxy- |
| | | carbonylamino-3-methylbutyrylamino)- |
| | | propionate |
| B-137 | a compound of the formula I | 4-fluorophenyl N-(1-(1-(4-cyanophenyl)- |
| | | ethanesulfonyl)but-2-yl)carbamate |
| B-138 | a compound of the formula I | dodine |
| B-139 | a compound of the formula I | iminoctadine |
| B-140 | a compound of the formula I | guazatine |
| B-141 | a compound of the formula I | kasugamycin |
| B-142 | a compound of the formula I | polyoxine |
| B-143 | a compound of the formula I | streptomycin |
| B-144 | a compound of the formula I | validamycin A |
| B-145 | a compound of the formula I | fentin salts |
| B-146 | a compound of the formula I | isoprothiolane |
| B-147 | a compound of the formula I | dithianon |
| B-148 | a compound of the formula I | edifenphos |
| B-149 | a compound of the formula I | fosetyl |
| B-150 | a compound of the formula I | fosetyl-aluminum |
| B-151 | a compound of the formula I | iprobenfos |
| B-152 | a compound of the formula I | pyrazophos |
| B-153 | a compound of the formula I | tolclofos-methyl |
| B-154 | a compound of the formula I | phosphorous acid and its salts |
| B-155 | a compound of the formula I | thiophanate methyl |
| B-156 | a compound of the formula I | chlorothalonil |
| B-157 | a compound of the formula I | dichlofluanid |
| B-158 | a compound of the formula I | tolylfluanid |
| B-159 | a compound of the formula I | flusulfamide |
| B-160 | a compound of the formula I | phthalide |
| B-161 | a compound of the formula I | hexachlorobenzene |
| B-162 | a compound of the formula I | pencycuron |
| B-163 | a compound of the formula I | quintozene |
| B-164 | a compound of the formula I | binapacryl |
| B-165 | a compound of the formula I | dinocap |
| B-166 | a compound of the formula I | dinobuton |
| B-167 | a compound of the formula I | Bordeaux mixture |
| B-168 | a compound of the formula I | copper acetate |
| B-169 | a compound of the formula I | copper hydroxide |
| B-170 | a compound of the formula I | copper oxychloride |
| B-171 | a compound of the formula I | basic copper sulfate |
| B-172 | a compound of the formula I | sulfur |
| B-173 | a compound of the formula I | spiroxamine |
| B-174 | a compound of the formula I | cyflufenamid |
| B-175 | a compound of the formula I | cymoxanil |
| B-176 | a compound of the formula I | metrafenone |

The active ingredients II, mentioned above as component 2, their preparation and their action against harmful fungi are generally known (cf.: http://www.hclrss.demon.co.uk/index.html); they are commercially available. The compounds named according to IUPAC, their preparation and their fungicidal action are likewise known [cf. EP-A 226 917; EP-A 10 28 125; EP-A 10 35 122; EP-A 12 01 648; WO 98/46608; WO 99/24413; WO 03/14103; WO 03/053145; WO 03/066609 and WO 04/049804].

The present invention further relates to the pharmaceutical use of the inventive azolopyrimidines of the formula I-I, in particular the azolopyrimidines of the formula I-I described in the above description as being preferred, and/or their pharmaceutically acceptable salts, in particular to their use for treating tumors in mammals for example man.

The invention thus also provides a medicament, especially for the treatment of tumors, comprising a compound of the formula I-I, and for the use of a compound of the formula I-I for producing a medicament, especially for the treatment of tumors.

### EXAMPLES

### A. Synthesis Examples

The methods described in the synthesis examples which follow have been utilized with appropriate modification of the starting compounds to obtain further compounds I. The compounds thus obtained are listed in the table which follows with physical data.

### Example 1

### Compound 732-A5

### {5-Chloro-6-[2,6-difluoro-4-(3-methoxypropoxy)phenyl]-[1,2,4]triazolo[1,5-a]pyrimidin-7-yl}-(2,2,2-trifluoroethyl)amine

0.16 g (6.75 mmol) of sodium hydride in 6 ml of tetrahydrofuran was admixed with 0.72 g (8 mmol) of 3-methoxy-1-propanol and stirred at room temperature. After approx. 15 min, 0.95 g (2.5 mmol) of [5-chloro-6-(2,4,6-trifluorophenyl)-[1,2,4]triazolo[1,5-a]pyrimidin-7-yl](2,2,2-trifluoroethyl)amine (prepared analogously to WO 1998/46607) was added and the mixture was stirred at 60° C. overnight. The reaction mixture was then cooled to room temperature, diluted with ethyl acetate and admixed with water and dilute hydrochloric acid. The phases were separated and the aqueous phase was extracted twice more with ethyl acetate. The combined organic phases were then dried and concentrated and the residue was purified by means of preparative MPLC with acetonitrile/water mixtures using RP 18 silica gel. This gave 0.15 g (13.6%) of the title compound as a yellow oil.

1H NMR (CDCl3, δ in ppm):

8.4 (s, 1H); 6.65 (d, 2H); 6.2 (t, broad, 1H); 4.2 (m, 2H); 4.1 (t, 2H); 3.55 (t, 2H); 3.4 (s, 3H); 2.1 (m, 2H)

### Example 2

### Compound 740-A8

### {5-Chloro-6-[4-(3-dimethylaminopropoxy)-2,6-difluorophenyl]-[1,2,4]triazolo[1,5-a]pyrimidin-7-yl}-(S)-(2,2,2-trifluoro-1-methylethyl)amine

0.32 g (13.4 mmol) of sodium hydride in 10 ml of tetrahydrofuran was admixed with 1.63 g (15.8 mmol) of 3-N,N-dimethylamino-1-propanol and stirred at room temperature. After approx. 15 min, 2 g (5 mmol) of 5-chloro-6-(2,4,6-trifluorophenyl)-[1,2,4]triazolo[1,5-a]pyrimidin-7-yl-(S)-(2,2,2-trifluoro-1-methylethyl)amine (prepared analogously to WO 1998/46607) were added and the mixture was stirred at 60° C. overnight. The reaction mixture was then cooled to room temperature and diluted with water, and the aqueous phase was extracted three times with ethyl acetate. The organic phase was concentrated by evaporation and the residue was filtered with suction through silica gel with ethyl acetate/methanol mixtures. The volatile components were then evaporated off first on a rotary evaporator and then on a Kugelrohr still under high vacuum. This gave 1.5 g (63%) of the title compound as a yellow oil.

1H NMR (CDCl3, δ in ppm):

8.4 (s, 1H); 6.65 (m, 2H); 5.9 (d, broad, 1H); 4.8 (m, 1H); 4.1 (t, 2H); 2.5 (t, 2H); 2.3 (s, 6H); 2.0 (m, 2H); 1.4 (d, 3H)

The remaining compounds of the formula I-I listed in Table I were obtained in an analogous manner.

TABLE I

| | |
|---|---|
| | I-I |
| 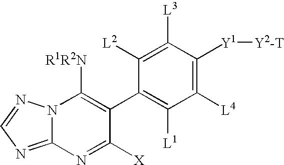 | |

| | | | | | | | | | | | Melting point [° C.]; 1H NMR [ppm] |
|---|---|---|---|---|---|---|---|---|---|---|---|
| Ex. No. | R¹ | R² | X | L¹ | L² | L³ | L⁴ | Y¹ | Y² | T | HPLC-MS (Rₜ, M + H)¹⁾ |
| I-1 | —CH₂—CF₃ | H | Cl | F | F | H | H | O | (CH₂)₃ | OCH₃ | 8.4 (s, 1H); 6.65 (d, 2H); 6.2 (t, breit, |
| (732-A5) | | | | | | | | | | | 1H); 4.2 (m, 2H); 4.1 (t, 2H); 3.55 (t, |
| | | | | | | | | | | | 2H); 3.4 (s, 3H); 2.1 (m, 2H) |
| I-2 | (S)-CH(CH₃)—CF₃ | H | Cl | F | F | H | H | O | (CH₂)₃ | N(CH₃)₂ | 8.4 (s, 1H); 6.65 (m, 2H); 5.9 (d, breit, |
| (740-A8) | | | | | | | | | | | 1H); 4.8 (m, 1H); 4.1 (t, 2H); 2.5 (t, 2H); |
| | | | | | | | | | | | 2.3 (s, 6H); 2.0 (m, 2H); 1.4 (d, 3H) |
| I-3 | —CH₂—CF₃ | H | Cl | F | F | H | H | O | (CH₂)₃ | N(CH₃)₂ | 8.35 (s, 1H); 6.6 (d, 2H); 4.3 (m, 2H); |
| (740-A5) | | | | | | | | | | | 4.05 (t, 2H); 2.5 (t, 2H); 2.3 (s, 6H); 2.0 |
| | | | | | | | | | | | (m, 2H) |
| I-4 | —CH₂—CF₃ | H | Cl | F | F | H | H | O | (CH₂)₃ | 4-methyl- | 8.35 (s, 1H); 6.6 (d, 2H); 4.45 (m, 2H); |
| (735-A5) | | | | | | | | | | piperazinyl | 4.05 (t, 2H); 2.5 (m, 10 H); 2.3 (s, 3H); |
| | | | | | | | | | | | 2.0 (m, 2H) |
| I-5 | (S)-CH(CH₃)—CF₃ | H | Cl | F | F | H | H | O | (CH₂)₃ | N(CH₃)₂ | 8.4 (s, 1H); 6.65 (m, 2H); 5.9 (d, breit, |
| (740-A8) | | | | | | | | | | | 1H); 4.8 (m, 1H); 4.1 (t, 2H); 2.5 (t, 2H); |
| | | | | | | | | | | | 2.3 (s, 6H); 2.0 (m, 2H); 1.4 (d, 3H) |
| I-6 | (S)-CH(CH₃)—CF₃ | H | Cl | F | F | H | H | O | (CH₂)₃ | OCH₃ | 170-175 |
| (732-A8) | | | | | | | | | | | |
| I-7 | (S)-CH(CH₃)—CF₃ | H | Cl | F | F | H | H | O | (CH₂)₃ | OH | 70-78 |
| (737-A8) | | | | | | | | | | | |
| I-8 | (S)-CH(CH₃)—CF₃ | H | Cl | F | F | H | H | O | (CH₂)₃ | NH(CH)₃ | 176-179 |
| (739-A8) | | | | | | | | | | | |
| I-9²⁾ | (S)-CH(CH₃)—CF₃ | H | Cl | F | F | H | H | O | (CH₂)₃ | NH₂ | Rₜ = 2.32 min |
| (738-A8) | | | | | | | | | | | M + H = 451.05 |
| I-10³⁾ | (S)-CH(CH₃)—CF₃ | H | Cl | F | F | H | H | O | (CH₂)₃ | NHCH₃ | Rₜ = 2.37 min |
| (739-A8) | | | | | | | | | | | M + H = 464.70 |
| I-11 | —CH₂—CF₃ | H | Cl | F | F | H | H | O | (CH₂)₂ | morpholin-1-yl | Rₜ = 2.18 min |
| | | | | | | | | | | | M + H = 493.15 |
| I-12 | —CH₂—CF₃ | H | Cl | F | F | H | H | O | (CH₂)₂ | piperidin-1-yl | Rₜ = 2.3 min |
| | | | | | | | | | | | M + H = 491.15 |
| I-13 | —CH₂—CF₃ | H | Cl | F | F | H | H | O | (CH₂)₂ | pyrrolidin-1-yl | 69-72 |
| | | | | | | | | | | | Rₜ = 2.25 min |
| | | | | | | | | | | | M + H = 477.15 |
| I-14 | —CH₂—CF₃ | H | Cl | F | F | H | H | O | (CH₂)₂ | pyrazol-1-yl | 197-200 |
| | | | | | | | | | | | Rₜ = 2.9 |
| | | | | | | | | | | | M + H = 474.05 |
| I-15 | —CH₂—CF₃ | H | Cl | F | F | H | H | O | (CH₂)₂ | 1,2,4-triazol-1-yl | Rₜ = 2.56 |
| | | | | | | | | | | | M + H = 475.05 |
| I-16 | —CH₂—CF₃ | H | Cl | F | F | H | H | O | (CH₂)₃ | morpholin-1-yl | Rₜ = 2.26 |
| | | | | | | | | | | | M + H = 507.15 |
| I-17 | —CH₂—CF₃ | H | Cl | F | F | H | H | O | (CH₂)₃ | piperidin-1-yl | Rₜ = 2.42 |
| A5) | | | | | | | | | | | M + H = 505.15 |
| I-18 | —CH₂—CF₃ | H | Cl | F | F | H | H | O | (CH₂)₃ | 2-oxo-pyrrolidin- | Rₜ = 2.91 |
| A5) | | | | | | | | | | 1-yl | M + H = 505.05 |
| I-19 | —CH₂—CF₃ | H | Cl | F | F | H | H | O | (CH₂)₃ | 2-oxo-pyrrolidin- | 181-184 |
| | | | | | | | | | | 1-yl | Rₜ = 2.79 |
| | | | | | | | | | | | M + H = 491.05 |
| I-20 | —CH₂—CF₃ | H | Cl | F | F | H | H | O | (CH₂)₃ | pyrrolidin-1-yl | Rₜ = 2.37 |
| | | | | | | | | | | | M + H = 491.15 |
| I-21 | —CH₂—CF₃ | H | Cl | F | F | H | H | O | (CH₂)₃ | pyrazol-1-yl | 62-66 |
| | | | | | | | | | | | Rₜ = 3.09 |
| | | | | | | | | | | | M + H = 488.05 |
| I-22 | —CH₂—CF₃ | H | Cl | F | F | H | H | O | (CH₂)₃ | —O—C(O)—N(CH₃)₂ | 117-119 |
| | | | | | | | | | | | Rₜ = 3.22 |
| | | | | | | | | | | | M + H = 509.05 |
| I-23 | —CH₂—CF₃ | H | Cl | F | F | H | H | O | (CH₂)₃ | —NH—C(O)— | 199-202 |
| | | | | | | | | | | N(CH₃)₂ | Rₜ = 2.8 |
| | | | | | | | | | | | M + H = 508.15 |
| I-24 | —CH₂—CF₃ | H | Cl | F | F | H | H | O | (CH₂)₃ | —N(CH₃)—C(O)— | Rₜ = 3.08 |
| | | | | | | | | | | N(CH₃)₂ | M + H = 522.15 |
| I-25 | (S)-CH(CH₃)—CF₃ | H | Cl | F | F | H | H | O | (CH₂)₂ | morpholin-1-yl | Rₜ = 2.32 |
| | | | | | | | | | | | M + H = 507.15 |
| I-26²⁾ | —CH₂—CF₃ | H | Cl | F | F | H | H | O | (CH₂)₃ | NH(CH₃) | Rₜ = 2.25 |
| (739-A5) | | | | | | | | | | | M + H = 451.05 |
| I-27²⁾ | —CH₂—CF₃ | H | Cl | F | F | H | H | O | (CH₂)₃ | —NH₂ | Rₜ = 2.19 |
| (738-A5) | | | | | | | | | | | M + H = 437.05 |
| I-28 | —CH₂—CF₃ | H | Cl | F | F | H | H | O | (CH₂)₃ | —N(CH₃)₂ | Rₜ = 2.39 |
| (740-A5) | | | | | | | | | | | M + H = 479.15 |
| I-29 | —CH₂—CF₃ | H | Cl | F | F | H | H | O | (CH₂)₃ | —N(CH₃)—C(O)— | Rₜ = 3.05 |
| | | | | | | | | | | CH₂—Cl | M + H = 527.05 |
| I-30 | —CH₂—CF₃ | H | Cl | F | F | H | H | O | (CH₂)₃ | —NH—C(O)—CHCl— | Rₜ = 3.09 |
| | | | | | | | | | | CH₃ | M + H = 527.05 |
| I-31 | —CH₂—CF₃ | H | Cl | F | F | H | H | O | (CH₂)₃ | —NH—C(O)—CH₂Cl | Rₜ = 2.93 |
| | | | | | | | | | | | M + H = 513.05 |
| I-32 | —CH₂—CF₃ | H | Cl | F | F | H | H | O | (CH₂)₃ | —N(CH₃)—C(O)— | Rₜ = 3.24 |
| | | | | | | | | | | CHCl—CH₃ | M + H = 541.05 |
| I-33 | —CH₂—CF₃ | H | Cl | F | F | H | H | O | (CH₂)₃ | —NH—C(O)—CH₂— | Rₜ = 2.83 |
| | | | | | | | | | | CH₃ | M + H = 493.15 |
| I-34 | —CH₂—CF₃ | H | Cl | F | F | H | H | O | (CH₂)₃ | 2,5-dioxo- | Rₜ = 2.92 |
| | | | | | | | | | | pyrrolidin-1-yl | M + H = 519.05 |
| I-35 | —CH₂—CF₃ | H | Cl | F | F | H | H | O | (CH₂)₃ | —N(CH₃)—C(O)— | Rₜ = 2.85 |
| | | | | | | | | | | CH₃ | M + H = 493.05 |
| I-36 | —CH₂—CF₃ | H | Cl | F | F | H | H | O | (CH₂)₃ | —N(CH₃)—C(O)H | Rₜ = 2.81 |
| | | | | | | | | | | | M + H = 479.05 |
| I-37 | —CH₂—CF₃ | H | Cl | F | F | H | H | O | (CH₂)₃ | —NH—C(O)H | 199-203 |
| | | | | | | | | | | | Rₜ = 2.65 |
| | | | | | | | | | | | M + H = 465.05 |
| I-38 | —CH₂—CF₃ | H | Cl | F | F | H | H | O | (CH₂)₃ | —NH—C(O)—CH₃ | 217-220 |
| | | | | | | | | | | | Rₜ = 2.68 |
| | | | | | | | | | | | M + H = 479.05 |
| I-39 | —CH₂—CF₃ | H | Cl | F | F | H | H | O | (CH₂)₂ | —O—N═C(CH₃)₂ | Rₜ = 3.36 |
| | | | | | | | | | | | M + H = 479.05 |
| I-40 | —CH₂—CF₃ | H | Cl | F | F | H | H | O | (CH₃)₃ | 2-oxo- | Rₜ = 2.64 |
| | | | | | | | | | | imidazolidin-1-yl | M + H = 492.05 |
| I-41 | —CH₂—CF₃ | H | Cl | F | F | H | H | O | (CH₂)₃ | 2,5- | Rₜ = 2.70 |
| | | | | | | | | | | dioxoimidazolidin- | M + H = 520.05 |
| | | | | | | | | | | 1-yl | |
| I-42 | —CH₂—CF₃ | H | Cl | F | F | H | H | O | (CH₂)₃ | NH—C(O)—CH₂— | 101-103 |
| | | | | | | | | | | NH—C(O)—O— | Rₜ = 3.08 |
| | | | | | | | | | | C(CH₃)₃ | M + H = 594.15 |
| I-43 | —CH₂—CF₃ | H | Cl | F | F | H | H | O | (CH₂)₃ | NH—C(O)—CH₂— | Rₜ = 2.29 |
| | | | | | | | | | | NH₂ | M + H = 494.15 |
| I-44 | —CH₂—CF₃ | CH₃ | Cl | F | F | H | H | O | (CH₂)₂ | OH | Rₜ = 2.75 |
| | | | | | | | | | | | M + H = 438.05 |

¹⁾Characterisation by HPLC-MS:
HPLC-column: RP-18 column (Chromolit Speed ROD of Merck KgaA, Germany), 50 mm x 4.6 mm
Eluent: acetronitrile + 0.1% trifluoroacetic acid (TFA)/water + 0.1% TFA in a gradient of from 5:95 to 95:5 in 5 minutes at 40° C., flow 1.8 ml/min
MS: Quadrupol Elektrospray Ionisation, 80 V (positive mode)
²⁾+HOOC—CF₃
³⁾+HOOC—CH₃

### Example 3

### Compound 739-A4, II-1

### Synthesis of {3-[4-(7-cyclohexyl-5-methyl-[1,2,4]triazolo[1,5-a]pyrimidin-6-yl)-3,5-difluoro-phenoxy]-propyl}-dimethyl-amine

### 3.1 6-(4-tert-Butoxy-2,6-difluoro-phenyl)-7-cyclohexyl-5-methyl-[1,2,4]triazolo[1,5-a]pyrimidin

37 g (0.1 mol) 7-cyclohexyl-5-methyl-6-(2,4,6-trifluorophenyl)-[1,2,4]triazolo[1,5,a]pyrimidine and 56 g (0.5 mol) potassium-t-butanolate in 600 ml dimethyl sultoxide were stirred overnight at room temperature. The reaction mixture was then diluted with water, acidified with dilute hydrochloric acid, and extracted thrice with methyl-t-butyl ether. The combined organic phases were extracted with water, dried over magnesium sulfate and concentrated. The residue was purified by means of column chromatography with cyclohexanolethyl acetate mixtures. This gave 26.2 g (61%) of the title compound as a yellow oil.

¹H-NMR (CDCl₃):

8.45 (s, 1H); 6.75 (d, 2H); 2.9 (s, broad, 1H); 2.45 (m, 5H); 1.8 (d, broad, 2H); 1.7 (m, 3H); 1.5 (s, 9H); 1.2 m, broad, 3H)

### 3.2 4-(7-cyclohexyl-5-methyl-[1,2,4]triazolo[1,5-a]pyrimidin-6-yl)-3,5-difluoro-phenol

26.2 g (0.071 mol) 6-(4-tert-butoxy-2,6-difluoro-phenyl)-7-cyclohexyl-5-methyl-[1,2,4]triazolo-[1,5-a]pyrimidine in 100 ml trifluoroacetic acid were stirred at room temperature for 4 hours. Then the reaction mixture was concentrated in vacuo and the residue was admixed with ethyl acetate and sodium bicarbonate solution. A solid precipitated which was filtered with suction, washed with hexane and dried. The mother liquor was concentrated, whereby a second crystal fraction was obtained. This gave a total of 20.8 g (79%) of the title compound as a light solid (mp: 290-292° C.).

¹H-NMR (DMSO-d₆):

10.95 (s, 1H); 6.75 (d, 2H); 2.95 (s, broad, 1H); 2.35 (m, 5H); 1.8 (d, broad, 2H); 1.6 (m, 3H); 1.2 (m, 3H)

### 3.3 {3-[4-(7-cyclohexyl-5-methyl-[1,2,4]triazolo[1,5-a]pyrimidin-6-yl)-3,5-difluoro-phenoxy]-propyl}-dimethyl-amine

1.05 g (3 mmol)-(7-cyclohexyl-5-methyl-[1,2,4]triazolo[1,5-a]pyrimidin-6-yl)-3,5-difluoro-phenol, 1.2 g triphenylphosphine und 0.41 g (4 mmol) dimethylaminopropanol in 10 ml tetrahydrofuran were admixed dropwise at room temperature with 0.75 g (4.5 mol) diethylazodicarboxylate. A slight warming occurred. After two hours, the reaction mixture was concentrated, whereupon a solid precipitated (triphenyl phosphine oxide). The precipitate was stirred with methyl-t-butyl ether, filtered with suction and discarded. The mother liquor was concentrated and the residue was purified by column chromatography with ethyl acetate/methanol mixtures. This gave 1.3 g of the title compound as a viscous mass which crystallized slowly (mp: 95-97° C.).

¹H-NMR (CDCl₃):

8.45 (s, 1H); 6.65 (d, 2H); 4.15 (t, 2H); 2.9 (s, broad, 1H); 2.5 (t, 2H); 2.4 (s, 3H); 2.3 (s, 6H); 2.0 (m, 2H); 1.8 (d, broad, 3H); 1.7 (m, 2H); 1.2 (m, 3H)

The remaining compounds of the formula I-II listed in Table 2 were obtained in an analogous manner.

TABLE II

| | |
|---|---|
| | I-II |
| 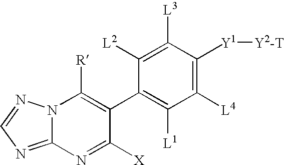 | |

| | | | | | | | | | | Melting point [° C.]; 1H NMR [ppm]; |
|---|---|---|---|---|---|---|---|---|---|---|
| Ex-No. | R | X | L¹ | L² | L³ | L⁴ | Y¹ | Y² | T | HPLC-MS (Rₜ, M + H)¹⁾ |
| II-1 | cyclohexyl | CH₃ | F | F | H | H | O | (CH₂)₃ | N(CH₃)₂ | 95-97 |
| | | | | | | | | | | Rₜ = 2.53 min |
| | | | | | | | | | | M + H = 430.25 |
| II-2 | cyclohexyl | CH₃ | F | F | H | H | O | (CH₂)₃ | NH₂ | Rₜ = 2.46 min |
| | | | | | | | | | | M + H = 402.15 |
| II-3 | cyclohexyl | CH₃ | F | F | H | H | O | (CH₂)₃ | NH(CH₃) | Rₜ = 2.49 min |
| | | | | | | | | | | M + H = 416.25 |
| II-4 | cyclohexyl | Cl | F | F | H | H | O | (CH₂)₃ | N(CH₃)₂ | Rₜ = 2.7 min |
| | | | | | | | | | | M + H = 450.15 |
| II-5²⁾ | cyclohexyl | Cl | F | F | H | H | O | (CH₂)₃ | N(CH₃)₂ | Rₜ = 2.79 min |
| | | | | | | | | | | M + H = 450.15 |
| II-6³⁾ | cyclohexyl | Cl | F | F | H | H | O | (CH₂)₃ | NH(CH3) | |

¹⁾s. Table I
²⁾+HCl
³⁾+HOOC—CF₃

### B. Biological Examples

### Active Ingredient Formulation

The active ingredients were formulated separately or together as a stock solution with 25 mg of active ingredient which had been made up to 10 ml with a mixture of acetone and/or DMSO and the emulsifier Wettol (wetter with emulsifying and dispersing action based on ethoxylated alkylphenols) in a solvent-emulsifier volume ratio of 99 to 1. Subsequently, the mixture was made up to 100 ml with water. This stock solution was diluted to the active ingredient concentration specified below with the solvent-emulsifier-water mixture described.

### Use Example 1

### Efficacy Against Early Blight on Tomatoes Caused by Alternaria solani

Leaves of potted tomato plants were sprayed to runoff point with an aqueous suspension having the active ingredient concentration specified below. The next day, the leaves were infected with an aqueous spore suspension of Alternaria solani in a 2% biomalt solution with a density of 0.17×10⁶ spores/ml. Subsequently, the plants were placed in a water vapor-saturated chamber at temperatures between 20 and 22° C. After 5 days, the disease on the untreated but infected control plants had developed to such an extent that the infection could be determined visually in %.

In this test, the plants which had been treated with compounds I-1, I-2, I-3, I-5, I-6, I-7, I-8, I-17, I-20, I-21, I-22, I-28, I-30, I-32, I-33, II-1, II-2, II-3, II-4 and II-5 at an active ingredient concentration of 250 ppm exhibited at most 15% infection, while the untreated plants were 90% infected.

### Use Example 2

### Efficacy Against Brown Rust of Wheat Caused by Erysiphe [syn. Blumeria] graminis form a specialis. tritici

Leaves of potted wheat seedlings were sprayed to runoff point with aqueous suspension in the active ingredient concentration specified below. The suspension or emulsion was prepared as described above. 24 hours after the sprayed layer had dried on, it was dusted with spores of brown rust of wheat (Erysiphe [syn. Blumeria] graminis form a specialis. tritici). The test plants were then placed in a greenhouse at temperatures between 20 and 24° C. and from 60 to 90% relative air humidity. After 7 days, the extent of brown rust development was determined visually in % infection of the total leaf area.

In this test, the plants treated with the compounds I-1, I-2, I-3, I-4 and I-5 at an active ingredient concentration of 250 ppm exhibited at most 10% infection, while the untreated plants were 90% infected.

### Use Example 3

### Activity Against Late Blight on Tomatoes Caused by Phytophthora infestans with Protective Treatment

Leaves of potted tomato plants were sprayed to runoff point with an aqueous suspension in the active ingredient concentration specified below. The next day, the leaves were infected with an aqueous sporangia suspension of Phytophthora infestans. Subsequently, the plants were placed in a water vapor-saturated chamber at temperatures between 18 and 20° C. After 6 days, the late blight on the untreated but infected control plants had developed to such an extent that the infection could be determined visually in %.

In this test, the plants treated with the compounds I-4, I-9, I-11, I-15, I-16, I-17, I-20, I-21, I-26, I-27, I-28, I-30, I-32, I-33, I-35, I-36, I-37, I-39, I-41, II-1, II-2, II-3, II-4 and II-5 at an active ingredient concentration of 250 ppm exhibited at most 15% infection, while the untreated plants were 90% infected.

### Use Example 4

### Efficacy Against Net Blotch of Barley Caused by Pyrenophora teres with 1-Day Protective Application

Leaves of potted barley seedlings were sprayed to runoff point with an aqueous suspension in the active ingredient concentration specified below. 24 hours after the sprayed layer had dried on, the test plants were inoculated with an aqueous spore suspension of Pyrenophora [syn. Drechslera] teres, the pathogen of net blotch. Subsequently, the test plants were placed in a greenhouse at temperatures between 20 and 24° C. and from 95 to 100% relative air humidity. After 6 days, the extent of disease development was determined visually in % infection of the total leaf area.

In this test, the plants treated with the compounds I-6, I-7, I-8, I-9, I-15, I-16, I-21, I-26, I-27, I-28, I-30, I-34, I-35, I-36, II-1, II-2, II-3, II-4 and II-5 at an active ingredient concentration of 250 ppm exhibited at most 15% infection, while the untreated plants were 90% infected.

### Use Example 5

### Protective Activity Against Puccinia recondita on Wheat (Wheat Leaf Rust)

Leaves of wheat seedlings in pots were sprayed until dripping wet with an aqueous suspension having a concentration of the active ingredient as given below. On the following day, the treated leaves were dusted with wheat leaf rust (Puccinia recondita) spores. Thereafter, the plants were placed for 24 hours into a chamber with high atmospheric humidity (90 to 95%) at 20 and 22° C. During this time, the spores germinated, and the germination tubes penetrated the plant tissue. On the next day, the test plants where placed into the greenhouse again and cultivated at temperatures of 20 to 22° C. and a relative atmospheric humidity of 65 to 70% for additional 7 days. Then, the extent of rust development on the leaves was determined visually in %.

In this test, the plants treated with the compounds I-11, I-13, I-22, I-34 and I-37 at an active ingredient concentration of 250 ppm exhibited at most 15% infection, while the untreated plants were 90% infected.

### Microtest

The active compounds were formulated separately as a stock solution having a concentration of 10 000 ppm in DMSO for the microtiter test (MT).

### Use Example 6

### Activity Against Gray Mould Caused by Botrytis cinera in a Microtiter Test

The stock solution of the active compound was pipetted onto a microtiter (MTP) and diluted to the concentration indicated below with an aqueous fungi nutrient medium based on malt. Subsequently, an aqueous spore suspension of Botrytis cinera was added. The plates were then placed in a humid chamber at a temperature of 18° C. and a relative humidity close to 100%. On the seventh day after inoculation, the MTPs were scanned with an absorption photometer at 405 nm.

The measured parameters were compared to the growth of the active-free control variant (100%) and the fungi-free and active-free blank value, to calculate the relative growth in % of the pathogens in the respective active compounds.

In these tests, the pathogens treated with 125 ppm of the active compounds I-10, I-12, I-14, I-18, I-19, I-23, I-26, I-30, I-31, I-38 and II-6 showed a relative growth of less than 15%.

### Use Example 7

### Activity Against Late Blight Caused by Phytophthora infestants in a Microtiter Test

The stock solution of the active compound was pipetted onto a microtiter (MTP) and diluted to the concentration indicated below with an aqueous fungi nutrient medium based on pea juice. Subsequently, an aqueous spore suspension of Phytophthora infestants was added. The plates were then placed in a humid chamber at a temperature of 18° C. and a relative humidity close to 100%. On the seventh day after inoculation, the MTPs were scanned with an absorption photometer at 405 nm.

The measured parameters were compared to the growth of the active-free control variant (100%) and the fungi-free and active-free blank value, to calculate the relative growth in % of the pathogens in the respective active compounds.

In these tests, the pathogens treated with 125 ppm of the active compounds I-12, I-18, I-19, I-31, I-38 and II-6 showed a relative growth of less than 10%.

### Use Example 8

### Activity Against Septoria Leaf Blotch Caused by Septoria tritici in a Microtiter Test

The stock solution of the active compound was pipetted onto a microtiter (MTP) and diluted to the concentration indicated below with an aqueous fungi nutrient medium based on malt. Subsequently, an aqueous spore suspension of Septoria tritici was added. The plates were then placed in a humid chamber at a temperature of 18° C. and a relative humidity close to 100%. On the seventh day after inoculation, the MTPs were scanned with an absorption photometer at 405 nm.

The measured parameters were compared to the growth of the active-free control variant (100%) and the fungi-free and active-free blank value, to calculate the relative growth in % of the pathogens in the respective active compounds.

In these tests, the pathogens treated with 125 ppm of the active compounds I-10, I-14 and I-26 showed a relative growth of less than 15%.

1-22. (canceled)

23: A method for controlling phytopathogenic harmful fungi, which comprises treating the fungi, or the materials, plants, the soil or seed to be protected from fungal infection, with an effective amount of a compound of formula I 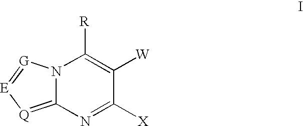
or an agriculturally acceptable salt thereof, where the substituents in the formula (I) are each defined as follows:
G, E, Q a) G is N; E is C—W² and Q is N or C—W³;
b) G is C—W¹; E is C—W² and Q is N; or
c) G is C—W¹; E is N and Q is C—W³;
W, W, W³ are each independently selected from the group consisting of hydrogen, halogen, cyano, nitro, C₁-C₄-alkyl, C₂-C₆-alkenyl, C₂-C₆-alkynyl, C₁-C₄-haloalkyl, hydroxy-C₁-C₄-alkyl, C₁-C₄-alkoxy-C₁-C₄-alkyl, C₂-C₆-haloalkenyl, C₂-C₆-haloalkynyl, C₃-C₆-cycloalkyl, C₃-C₆-halocycloalkyl, C₁-C₄-alkoxy, C₁-C₄-haloalkoxy, C₁-C₄-alkylthio, C₁-C₄-alkylsulfinyl, C₁-C₄-alkylsulfonyl, formyl, thiocarbamoyl, C₁-C₄-alkylcarbonyl, C₁-C₄-alkoxycarbonyl, C₁-C₄-alkylaminocarbonyl, aminocarbonyl, di(C₁-C₄-alkyl)aminocarbonyl, C₁-C₄-alkoximinoalkyl, hydroximinoalkyl, and CR¹⁰R¹¹OR¹², C(R¹³)═NR¹⁴;
R¹⁰, R¹¹, R¹² are each independently hydrogen, C₁-C₈-alkyl, C₃-C₆-cycloalkyl, C₁-C₈-alkoxy-C₁-C₈-alkyl, C₂-C₈-alkenyl, C₂-C₈-alkynyl, or benzyl;
R¹¹ and R¹² may together be oxy-C₁-C₅-alkyleneoxy in which the carbon chain may be substituted by from one to three groups selected from the group consisting of methyl, ethyl, hydroxyl, methoxy, ethoxy, hydroxymethyl, methoxymethyl, and ethoxymethyl;
R¹³ is hydrogen or C₁-C₈-alkyl;
R¹⁴ is C₁-C₈-alkyl, C₃-C₆-cycloalkyl, phenyl, or phenylamino, where the phenyl groups may be substituted by from one to five R^{b} groups;
R is NR¹R², C₃-C₆-cycloalkyl or C₃-C₁₂-halocycloalkyl; where R may comprise one, two, three or four identical or different R^{a} groups which are each independently selected from:
R^{a} is R^{b}, carboxyl, OC(O)OR^{Π} or C₁-C₆-alkylthio;
R^{b} is R^{c}, hydroxyl, C₁-C₆-alkoxy, C₂-C₆-alkenyloxy, C₃-C₆-alkynyloxy, C₃-C₆-cycloalkoxy or C₃-C₆-cycloalkenyloxy;
R^{c} is hydrogen, cyano, C₁-C₆-alkyl, C₂-C₆-alkenyl, C₂-C₆-alkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, amino, C₁-C₆-alkylamino, di-C₁-C₆-alkylamino, aminocarbonyl, C(O)NHR^{Π}, C(O)NR^{Π}₂, phenyl, naphthyl, five-, six-, seven-, eight-, nine- or ten-membered saturated, partially unsaturated or aromatic heterocycle comprising one, two, three or four heteroatoms selected from the group consisting of O, N and S,
or two R^{a}, R^{b}, R^{C}, R^{Π}, R^{d} and/or R^{dd} groups form, together with the atom or the atoms to which they are bonded, a 3- to 12-membered saturated, partially unsaturated or aromatic ring which is carbocyclic or comprises from one to four heteroatoms selected from the group consisting of N, O and S and which is unsubstituted or substituted by from 1 to 4 R^{d} radicals, in the case of halogen even up to the maximum number;
R^{Π} is C₁-C₈-alkyl, C₃-C₈-alkenyl, C₃-C₈-alkynyl, C₃-C₆-cycloalkyl or C₃-C₆-cycloalkenyl;
where the aliphatic, alicyclic or aromatic groups in the aforementioned R^{a}, R^{b}, R^{c} and R^{Π} groups may in turn be partially or fully halogenated and/or bear one, two or three R^{d} groups:
R^{d} is halogen, cyano, nitro, hydroxyl, mercapto, amino, carboxyl, alkyl, haloalkyl, alkenyl, alkynyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms; cycloalkyl, cycloalkoxy, heterocyclyl, heterocyclyloxy, where the cyclic systems comprise from 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals comprise preferably from 6 to 10 ring members, the hetaryl radicals 5 or 6 ring members, where the cyclic systems are partially or completely halogenated and/or may bear from 1 to 3 R^{dd} groups;
R^{dd} is halogen, cyano, nitro, hydroxyl, mercapto, amino, carboxyl, alkyl, haloalkyl, alkenyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms;
R¹ is C₁-C₁₂-haloalkyl, C₂-C₁₂-haloalkenyl, or C₂-C₁₂-haloalkynyl;
R² is H, R¹, C₁-C₁₂-alkyl, C₂-C₁₂-alkenyl, C₂-C₁₂-alkynyl, C₃-C₈-cycloalkyl, C₃-C₈-halocycloalkyl, C₃-C₆-cycloalkenyl, C₃-C₆-halocycloalkenyl, C₁-C₈-alkoxy, C₂-C₈-alkenyloxy, C₂-C₈-alkynyloxy, C₃-C₈-cycloalkoxy, NH₂, C₁-C₈-alkylamino, di-C₁-C₈-alkylamino, phenyl, naphthyl or a five- or six-membered saturated, partially unsaturated or aromatic heterocycle comprising one, two, three or four heteroatoms selected from the group consisting of O, N and S, or Z—Y—(CR⁷R⁸)ₚ—(CR⁵R⁶)_{q}—CR³R⁴—# where # is the bonding site to the nitrogen atom and:
R³, R⁴, R⁵, R⁶, R⁷, R⁸ are each independently hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl, C₃-C₆-cycloalkenyl, C₃-C₆-halocycloalkenyl, phenyl, naphthyl or a five- or six-membered saturated, partially unsaturated or aromatic heterocycle comprising one, two, three or four heteroatoms selected from the group consisting of O, N and S,
R⁵ may also, with R³ or R⁷, together with the atoms to which these radicals are bonded, form a five-, six-, seven-, eight-, nine- or ten-membered saturated or partially unsaturated ring which, as well as carbon atoms, may comprise one, two or three heteroatoms selected from the group consisting of O, N and S as ring members and/or may bear one or more substituents R^{a};
R³ with R⁴, R⁵ with R⁶, R⁷ with R⁸ may each together also form carbonyl groups by being oxygen and form spiro groups by forming a C₂-C₅-alkylene or alkenylene, alkynylene chain which may be interrupted by one, two or three heteroatoms selected from the group consisting of O, N and S;
R¹ to R⁸ may each independently bear one, two, three or four identical or different R^{a} groups;
Y is oxygen or sulfur;
Z is hydrogen, carboxyl, formyl, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, C(NR^{A})SR^{Π}, C(S)R^{Π}, C(NR^{Π})NR^{A}R^{B}, C(NR^{Π})R^{A}, C(NR^{Π})OR^{A}, C(O)NR^{A}R^{B}, C(S)NR^{A}R^{B}, C₁-C₈-alkylsulfinyl, C₁-C₈-alkylthio, C₁-C₈-alkylsulfonyl, C(O)—C₁-C₄-alkyleneNR^{A}C(NR^{Π})NR^{A}R^{B},
C(S)—C₁-C₄-alkylene-NR^{A}C(NR^{Π})NR^{A}R^{B},
C(NR^{Π})—C₁-C₄-alkylene-NR^{A}C(NR^{Π})NR^{A}R^{B}, phenyl, naphthyl, five-, six-, seven-, eight-, nine- or ten-membered saturated, partially unsaturated or aromatic heterocycle which comprises one, two, three or four heteroatoms selected from the group consisting of O, N and S and is bonded directly or via a carbonyl, thiocarbonyl, C₁-C₄-alkylcarbonyl or C₁-C₄-alkylthiocarbonyl group; where the carbon chains in the Z group may be substituted by one or more R^{b} groups;
R^{A}, R^{B} are each independently hydrogen, C₂-alkenyl, C₂-alkynyl or one of the groups mentioned for R^{Π};
R^{A} and R^{B}, together with the nitrogen atom to which they are bonded, or R^{A} and R^{Π} together with the carbon atoms and heteroatoms via which they are bonded, may also form a five- or six-membered saturated, partly unsaturated or aromatic ring which, as well as carbon atoms, may comprise one, two or three further heteroatoms selected from the group consisting of O, N and S as ring members and/or may bear one or more substituents R^{a};
or
Z may also, with R⁶ or R⁸, form a five- or six-membered saturated or partly unsaturated ring which, as well as carbon atoms and Y, may comprise one or two further heteroatoms selected from the group consisting of N and S as ring members and/or may bear one or more substituents R^{a} as defined below;
the Z group may be partially or completely halogenated and/or bear one, two or three R^{b} groups;
W is phenyl or five- or six-membered heteroaryl which, as well as carbon atoms, comprises one, two or three further heteroatoms selected from the group consisting of O, N and S as ring members, where the ring systems, as well as Lₘ groups, bear at least one substituent P¹,
P¹ is Y¹—Y¹-T;
Y¹ is CR^{a}R^{a′}, C(O)O, C(O)NR^{b}, O, NR^{b} or S(O)ᵣ;
Y² is C₃-C₈-alkylene, C₂-C₈-alkenylene, C₂-C₈-alkynylene, C₃-C₈-alkylene-(Y³—(C₂-C₄-alkylene))ₛ, C₃-C₈-alkenylene-(Y³—(C₂-C₄-alkylene))ₛ, C₃-C₈-alkynylene-(Y³—(C₂-C₄-alkylene))ₛ, and, if R is C₃-C₆-cycloalkyl or C₃-C₁₂-halocycloalkyl and/or Y¹ is CR^{a}R^{a′}, C(O)O, CONR^{b}, NR^{b} or S(O)ᵣ, is also C₁-C₂-alkylene, and/or, if T is OC(O)R^{a}, NR^{b}R^{b′}, C(O)NR^{b}R^{b′}, C(NOR^{c})R^{a} or T¹-C(=T²)-T³, is also C₁-C₂-alkylene;
Y³ is O, S, or NR^{b}
r is 0, 1 or 2 is 1 or 2,
T is OR^{c}, OC(O)R^{a}, NR^{b}R^{b′}, C(O)NR^{b}R^{b′}, C(NOR^{c})R^{a}, or T¹-C(=T²)-T³, and, if R is defined as NR¹R² or C₃-C₁₂-halocycloalkyl, is also C(O)OR^{c} or C(OR^{c})₂R^{a};
T¹ is O, or NR^{b};
T² is O, S, or NR^{b};
T³ is R^{a′}, OR^{c}, SR^{c}, or NR^{b}R^{b′},
where R^{a′} and R^{b}, independently of R^{a} and R^{b} respectively, are each as defined for these radicals;
L is halogen, hydroxyl, cyanato (OCN), cyano, nitro, C₁-C₈-alkyl, C₁-C₈-haloalkyl, C₂-C₁₀-alkenyl, C₂-C₁₀-haloalkenyl, C₂-C₁₀-alkynyl, C₃-C₆-cycloalkyl, C₃-C₆-halocycloalkyl, C₃-C₆-cycloalkenyl, C₁-C₈-alkoxy, C₁-C₈-haloalkoxy, C₂-C₁₀-alkenyloxy, C₂-C₁₀-alkynyloxy, C₃-C₆-cycloalkyloxy, C₃-C₆-cycloalkenyloxy, amino, C₁-C₄-alkylamino, di-(C₁-C₄)-alkylamino, C₁-C₄-alkylcarbonylamino, C(O)—R^{Φ}, C(S)—R^{Φ}, S(O)ₙ—R^{Φ}; C₁-C₈-alkoxyimino-(C₁-C₈)-alkyl, C₂-C₁₀-alkenyloxyimino-(C₁-C₈)-alkyl, C₂-C₁₀-alkynyloxyimino-(C₁-C₈)-alkyl, C₂-C₁₀-alkynylcarbonyl, C₃-C₆-cycloalkylcarbonyl, or a five-, six-, seven-, eight-, nine- or ten-membered saturated, partly unsaturated or aromatic heterocycle comprising one, two, three or four heteroatoms selected from the group consisting of O, N and S;
R^{Φ} is hydrogen, C₁-C₄-alkyl, C₁-C₂-haloalkyl, C₁-C₄-alkoxy, C₂-C₄-alkenyloxy, C₂-C₄-alkynyloxy, amino, C₁-C₄-alkylamino, or di-C₁-C₄-alkylamino; where the R¹ groups may be substituted by one, two or three identical or different R^{b} groups as defined above;
n is zero, 1 or 2;
m is zero, 1, 2, 3, 4 or 5 and
X is halogen, cyano, C₁-C₄-alkyl, C₁-C₄-haloalkyl, C₁-C₄-alkoxy or C₁-C₄-haloalkoxy.

24: The method of claim 23, wherein said compounds have the formula I.a:

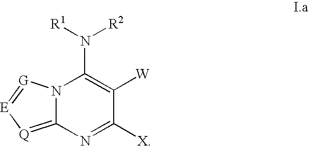

25: The method of claim 23, wherein said compounds have the formula I.b 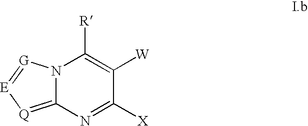
wherein R′ is an R group according to claim 23 bonded via carbon.

26: The method of claim 23, wherein X in formula I is halogen.

27: The method of claim 23, wherein X in formula I is methyl.

28: The method of claim 23, wherein X in formula I is methoxy or cyano.

29: The method of claim 23, wherein W is phenyl substituted by P¹ and Lₘ.

30: The method of claim 23, wherein said compound has the formula

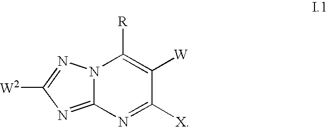

31: The method of claim 23, wherein said compound has the formula

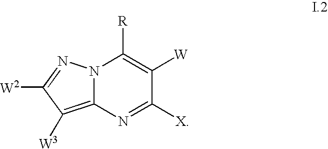

32: The method of claim 23, wherein said compound has the formula

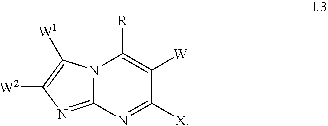

33: The method of claim 23, wherein said compound has the formula

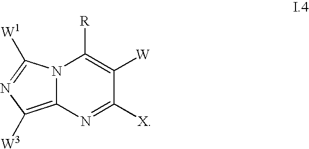

34: The method of claim 23, wherein P¹ is a group bonded via oxygen.

35: A composition comprising a solid or liquid carrier and a compound of formula I of claim 23 and a further active agrochemical ingredient.

36: Seed comprising a compound of formula I of claim 23 in an amount of from 1 to 1000 g per 100 kg.

37: A compound of formula I 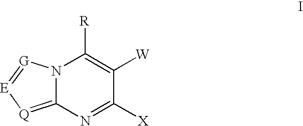
or an agriculturally acceptable salt thereof, wherein the substituents in the formula (I) are each defined as follows:
G, E, Q a) G is N; E is C—W² and Q is N or C—W³;
b) G is C—W¹; E is C—W² and Q is N; or
c) G is C—W¹; E is N and Q is C—W³;
W¹, W², W³ are each independently selected from the group consisting of hydrogen, halogen, cyano, nitro, C₁-C₄-alkyl, C₂-C₆-alkenyl, C₂-C₆-alkynyl, C₁-C₄-haloalkyl, hydroxy-C₁-C₄-alkyl, C₁-C₄-alkoxy-C₁-C₄-alkyl, C₂-C₆-haloalkenyl, C₂-C₆-haloalkynyl, C₃-C₆-cycloalkyl, C₃-C₆-halocycloalkyl, C₁-C₄-alkoxy, C₁-C₄-haloalkoxy, C₁-C₄-alkylthio, C₁-C₄-alkylsulfinyl, C₁-C₄-alkylsulfonyl, formyl, thiocarbamoyl, C₁-C₄-alkylcarbonyl, C₁-C₄-alkoxycarbonyl, C₁-C₄-alkylaminocarbonyl, aminocarbonyl, di(C₁-C₄-alkyl)aminocarbonyl, C₁-C₄-alkoximinoalkyl, hydroximinoalkyl, and CR¹⁰R¹¹OR¹², C(R¹³)═NR¹⁴;
R¹⁰, R¹¹, R¹² are each independently hydrogen, C₁-C₈-alkyl, C₃-C₆-cycloalkyl, C₁-C₈-alkoxy-C₁-C₈-alkyl, C₂-C₈-alkenyl, C₂-C₈-alkynyl, or benzyl;
R¹¹ and R¹² may together be oxy-C₁-C₅-alkyleneoxy in which the carbon chain may be substituted by from one to three groups selected from the group consisting of methyl, ethyl, hydroxyl, methoxy, ethoxy, hydroxymethyl, methoxymethyl, and ethoxymethyl;
R¹³ is hydrogen or C₁-C₈-alkyl;
R¹⁴ is C₁-C₈-alkyl, C₃-C₆-cycloalkyl, phenyl, or phenylamino, where the phenyl groups may be substituted by from one to five R^{b} groups;
R is NR¹R², C₃-C₆-cycloalkyl or C₃-C₁₂-halocycloalkyl; where R may comprise one, two, three or four identical or different R^{a} groups which are each independently selected from:
R^{a} is R^{b}, carboxyl, OC(O)OR^{Π} or C₁-C₆-alkylthio;
R^{b} is R^{c}, hydroxyl, C₁-C₆-alkoxy, C₂-C₆-alkenyloxy, C₃-C₆-alkynyloxy, C₃-C₆-cycloalkoxy or C₃-C₆-cycloalkenyloxy;
R^{c} is hydrogen, cyano, C₁-C₆-alkyl, C₂-C₆-alkenyl, C₂-C₆-alkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, amino, C₁-C₆-alkylamino, di-C₁-C₆-alkylamino, aminocarbonyl, C(O)NHR^{Π}, C(O)NR^{Π}₂, phenyl, naphthyl, five-, six-, seven-, eight-, nine- or ten-membered saturated, partially unsaturated or aromatic heterocycle comprising one, two, three or four heteroatoms selected from the group consisting of O, N and S,
or two R^{a}, R^{b}, R^{C}, R^{Π}, R^{d} and/or R^{dd} groups form, together with the atom or the atoms to which they are bonded, a 3- to 12-membered saturated, partially unsaturated or aromatic ring which is carbocyclic or comprises from one to four heteroatoms selected from the group consisting of N, O and S and which is unsubstituted or substituted by from 1 to 4 R^{d} radicals, in the case of halogen even up to the maximum number;
R^{Π} is C₁-C₈-alkyl, C₃-C₈-alkenyl, C₃-C₈-alkynyl, C₃-C₆-cycloalkyl or C₃-C₆-cycloalkenyl;
where the aliphatic, alicyclic or aromatic groups in the aforementioned R^{a}, R^{b}, R^{c} and R^{Π} groups may in turn be partially or fully halogenated and/or bear one, two or three R^{d} groups:
R^{d} is halogen, cyano, nitro, hydroxyl, mercapto, amino, carboxyl, alkyl, haloalkyl, alkenyl, alkynyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms; cycloalkyl, cycloalkoxy, heterocyclyl, heterocyclyloxy, where the cyclic systems comprise from 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals comprise preferably from 6 to 10 ring members, the hetaryl radicals 5 or 6 ring members, where the cyclic systems are partially or completely halogenated and/or may bear from 1 to 3 R^{dd} groups;
R^{dd} is halogen, cyano, nitro, hydroxyl, mercapto, amino, carboxyl, alkyl, haloalkyl, alkenyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms;
R¹ is C₁-C₁₂-haloalkyl, C₂-C₁₂-haloalkenyl, or C₂-C₁₂-haloalkynyl;
R² is H, R¹, C₁-C₁₂-alkyl, C₂-C₁₂-alkenyl, C₂-C₁₂-alkynyl, C₃-C₈-halocycloalkyl, C₃-C₆-cycloalkenyl, C₃-C₆-halocycloalkenyl, C₁-C₈-alkoxy, C₂-C₈-alkenyloxy, C₂-C₈-alkynyloxy, C₃-C₈-cycloalkoxy, NH₂, C₁-C₈-alkylamino, di-C₁-C₈-alkylamino, phenyl, naphthyl or a five- or six-membered saturated, partially unsaturated or aromatic heterocycle comprising one, two, three or four heteroatoms selected from the group consisting of O, N and S, or Z—Y—(CR⁷R⁸)ₚ—(CR⁵R⁶)_{q}—CR³R⁴-# where # is the bonding site to the nitrogen atom and:
R³, R⁴, R⁵, R⁶, R⁷, R⁸ are each independently hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl, C₃-C₆-cycloalkenyl, C₃-C₆-halocycloalkenyl, phenyl, naphthyl or a five- or six-membered saturated, partially unsaturated or aromatic heterocycle comprising one, two, three or four heteroatoms selected from the group consisting of O, N and S,
R⁵ may also, with R³ or R⁷, together with the atoms to which these radicals are bonded, form a five-, six-, seven-, eight-, nine- or ten-membered saturated or partially unsaturated ring which, as well as carbon atoms, may comprise one, two or three heteroatoms selected from the group consisting of O, N and S as ring members and/or may bear one or more substituents R^{a};
R³ with R⁴, R⁵ with R⁶, R⁷ with R⁸ may each together also form carbonyl groups by being oxygen and form spiro groups by forming a C₂-C₅-alkylene or alkenylene, alkynylene chain which may be interrupted by one, two or three heteroatoms selected from the group consisting of O, N and S;
R¹ to R⁸ may each independently bear one, two, three or four identical or different R^{a} groups;
Y is oxygen or sulfur;
Z is hydrogen, carboxyl, formyl, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, C(NR^{A})SR^{Π}, C(S)R^{Π}, C(NR^{Π})NR^{A}R^{B}, C(NR^{Π})R^{A}, C(NR^{Π})OR^{A}, C(O)NR^{A}R^{B}, C(S)NR^{A}R^{B}, C₁-C₈-alkylsulfinyl, C₁-C₈-alkylthio, C₁-C₈-alkylsulfonyl, C(O)—C₁-C₄-alkylene-NR^{C}(NR^{Π})NR^{A}R^{B},
C(S)—C₁-C₄-alkylene-NR^{A}C(NR^{Π})NR^{A}R^{B},
C(NR^{Π})—C₁-C₄-alkylene-NR^{A}C(NR^{Π})NR^{A}R^{B}, phenyl, naphthyl, five-, six-, seven-, eight-, nine- or ten-membered saturated, partially unsaturated or aromatic heterocycle which comprises one, two, three or four heteroatoms selected from the group consisting of O, N and S and is bonded directly or via a carbonyl, thiocarbonyl, C₁-C₄-alkylcarbonyl or C₁-C₄-alkylthiocarbonyl group; where the carbon chains in the Z group may be substituted by one or more R^{b} groups;
R^{A}, R^{B} are each independently hydrogen, C₂-alkenyl, C₂-alkynyl or one of the groups mentioned for R^{Π};
R^{A} and R^{B}, together with the nitrogen atom to which they are bonded, or R^{A} and R^{Π} together with the carbon atoms and heteroatoms via which they are bonded, may also form a five- or six-membered saturated, partly unsaturated or aromatic ring which, as well as carbon atoms, may comprise one, two or three further heteroatoms selected from the group consisting of O, N and S as ring members and/or may bear one or more substituents R^{a};
or
Z may also, with R⁶ or R⁸, form a five- or six-membered saturated or partly unsaturated ring which, as well as carbon atoms and Y, may comprise one or two further heteroatoms selected from the group consisting of N and S as ring members and/or may bear one or more substituents R^{a} as defined below;
the Z group may be partially or completely halogenated and/or bear one, two or three R^{b} groups;
W is phenyl or five- or six-membered heteroaryl which, as well as carbon atoms, comprises one, two or three further heteroatoms selected from the group consisting of O, N and S as ring members, where the ring systems, as well as Lₘ groups, bear at least one substituent P¹,
P¹ is Y¹—Y²-T;
Y¹ is C(O)O, C(O)NR^{A}, or S(O)ₜ,
is 1 or 2,
Y² is C₃-C₈-alkylene, C₂-C₈-alkenylene, C₂-C₈-alkynylene, C₃-C₈-alkylene-(Y³—(C₂-C₄-alkylene))ₛ, C₃-C₈-alkenylene-(Y³—(C₂-C₄-alkylene))ₛ, C₃-C₈-alkynylene-(Y³—(C₂-C₄-alkylene))ₛ, and, if R is C₃-C₆-cycloalkyl or C₃-C₁₂-halocycloalkyl and/or Y¹ is CR^{a}R^{a′}, C(O)O, CONR^{b}, NR^{b} or S(O)ᵣ, is also C₁-C₂-alkylene, and/or, if T is OC(O)R^{a}, NR^{b}R^{b′}, C(O)NR^{b}R^{b′}, C(NOR^{c})R^{a} or T¹-C(=T²)-T³, is also C₁-C₂-alkylene;
Y³ is O, S, or NR^{b}
r is 0, 1 or 2
s is 1 or 2,
T is OR^{c}, OC(O)R^{a}, NR^{b}R^{b′}, C(O)NR^{b}R^{b′}, C(NOR^{c})R^{a}, or T¹-C(=T²)-T³, and, if R is defined as NR¹R² or C₃-C₁₂-halocycloalkyl, is also C(O)OR^{c} or C(OR^{c})₂R^{a};
T¹ is O, or NR^{b};
T² is O, S, or NR^{b};
T³ is R^{a}, OR^{c}, SR^{c}, or NR^{b}R^{b′},
where R^{a′} and R^{b′}, independently of R^{a} and R^{b} respectively, are each as defined for these radicals;
L is halogen, hydroxyl, cyanato (OCN), cyano, nitro, C₁-C₈-alkyl, C₁-C₈-haloalkyl, C₂-C₁₀-alkenyl, C₂-C₁₀-haloalkenyl, C₂-C₁₀-alkynyl, C₃-C₆-cycloalkyl, C₃-C₆-halocycloalkyl, C₃-C₆-cycloalkenyl, C₁-C₈-alkoxy, C₁-C₈-haloalkoxy, C₂-C₁₀-alkenyloxy, C₂-C₁₀-alkynyloxy, C₃-C₆-cycloalkyloxy, C₃-C₆-cycloalkenyloxy, amino, C₁-C₄-alkylamino, di-(C₁-C₄)-alkylamino, C₁-C₄-alkylcarbonylamino, C(O)—R^{Φ}, C(S)—R^{Φ}, S(O)ₙ—R^{Φ}; C₁-C₈-alkoxyimino-(C₁-C₈)-alkyl, C₂-C₁₀-alkenyloxyimino-(C₁-C₈)-alkyl, C₂-C₁₀-alkynyloxyimino-(C₁-C₈)-alkyl, C₂-C₁₀-alkynylcarbonyl, C₃-C₆-cycloalkylcarbonyl, or a five-, six-, seven-, eight-, nine- or ten-membered saturated, partly unsaturated or aromatic heterocycle comprising one, two, three or four heteroatoms selected from the group consisting of O, N and S;
R^{Φ} is hydrogen, C₁-C₄-alkyl, C₁-C₂-haloalkyl, C₁-C₄-alkoxy, C₂-C₄-alkenyloxy, C₂-C₄-alkynyloxy, amino, C₁-C₄-alkylamino, or di-C₁-C₄-alkylamino; where the R^{Φ} groups may be substituted by one, two or three identical or different R^{b} groups as defined above;
n is zero, 1 or 2;
m is zero, 1, 2, 3, 4 or 5 and
X is halogen, cyano, C₁-C₄-alkyl, C₁-C₄-haloalkyl, C₁-C₄-alkoxy or C₁-C₄-haloalkoxy.

38: A compound of formula I 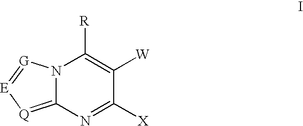
or an agriculturally acceptable salt thereof, wherein the substituents in the formula (I) are each defined as follows:
G, E, Q a) G is N; E is C—W² and Q is N or C—W³;
b) G is C—W¹; E is C—W² and Q is N; or
c) G is C—W¹; E is N and Q is C—W³;
W¹, W², W³ are each independently selected from the group consisting of hydrogen, halogen, cyano, nitro, C₁-C₄-alkyl, C₂-C₆-alkenyl, C₂-C₆-alkynyl, C₁-C₄-haloalkyl, hydroxy-C₁-C₄-alkyl, C₁-C₄-alkoxy-C₁-C₄-alkyl, C₂-C₆-haloalkenyl, C₂-C₆-haloalkynyl, C₃-C₆-cycloalkyl, C₃-C₆-halocycloalkyl, C₁-C₄-alkoxy, C₁-C₄-haloalkoxy, C₁-C₄-alkylthio, C₁-C₄-alkylsulfinyl, C₁-C₄-alkylsulfonyl, formyl, thiocarbamoyl, C₁-C₄-alkylcarbonyl, C₁-C₄-alkoxycarbonyl, C₁-C₄-alkylaminocarbonyl, aminocarbonyl, di(C₁-C₄-alkyl)aminocarbonyl, C₁-C₄-alkoximinoalkyl, hydroximinoalkyl, and CR¹⁰R¹¹OR¹², C(R¹³)═NR¹⁴;
R¹⁰, R¹¹, R¹² are each independently hydrogen, C₁-C₈-alkyl, C₃-C₆-cycloalkyl, C₁-C₈-alkoxy-C₁-C₈-alkyl, C₂-C₈-alkenyl, C₂-C₈-alkynyl, or benzyl;
R¹¹ and R¹² may together be oxy-C₁-C₅-alkyleneoxy in which the carbon chain may be substituted by from one to three groups selected from the group consisting of methyl, ethyl, hydroxyl, methoxy, ethoxy, hydroxymethyl, methoxymethyl, and ethoxymethyl;
R¹³ is hydrogen or C₁-C₈-alkyl;
R¹⁴ is C₁-C₈-alkyl, C₃-C₆-cycloalkyl, phenyl, or phenylamino, where the phenyl groups may be substituted by from one to five R^{b} groups;
R is NR¹R², C₃-C₆-cycloalkyl or C₃-C₁₂-halocycloalkyl; where R^{a} may comprise one, two, three or four identical or different R^{a} groups which are each independently selected from:
R^{a} is R^{b}, carboxyl, OC(O)OR^{Π} or C₁-C₆-alkylthio;
R^{b} is R^{c}, hydroxyl, C₁-C₆-alkoxy, C₂-C₆-alkenyloxy, C₃-C₆-alkynyloxy, C₃-C₆-cycloalkoxy or C₃-C₆-cycloalkenyloxy;
R^{c} is hydrogen, cyano, C₁-C₆-alkyl, C₂-C₆-alkenyl, C₂-C₆-alkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, amino, C₁-C₆-alkylamino, di-C₁-C₆-alkylamino, aminocarbonyl, C(O)NHR^{Π}, C(O)NR^{Π}₂, phenyl, naphthyl, five-, six-, seven-, eight-, nine- or ten-membered saturated, partially unsaturated or aromatic heterocycle comprising one, two, three or four heteroatoms selected from the group consisting of O, N and S,
or two R^{a}, R^{b}, R^{C}, R^{Π}, R^{d} and/or R^{dd} groups form, together with the atom or the atoms to which they are bonded, a 3- to 12-membered saturated, partially unsaturated or aromatic ring which is carbocyclic or comprises from one to four heteroatoms selected from the group consisting of N, O and S and which is unsubstituted or substituted by from 1 to 4 R^{d} radicals, in the case of halogen even up to the maximum number;
R^{Π} is C₁-C₈-alkyl, C₃-C₈-alkenyl, C₃-C₈-alkynyl, C₃-C₆-cycloalkyl or C₃-C₆-cycloalkenyl;
where the aliphatic, alicyclic or aromatic groups in the aforementioned R^{a}, R^{b}, R^{c} and R^{Π} groups may in turn be partially or fully halogenated and/or bear one, two or three R^{d} groups:
R^{d} is halogen, cyano, nitro, hydroxyl, mercapto, amino, carboxyl, alkyl, haloalkyl, alkenyl, alkynyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms; cycloalkyl, cycloalkoxy, heterocyclyl, heterocyclyloxy, where the cyclic systems comprise from 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals comprise preferably from 6 to 10 ring members, the hetaryl radicals 5 or 6 ring members, where the cyclic systems are partially or completely halogenated and/or may bear from 1 to 3 R^{dd} groups;
R^{dd} is halogen, cyano, nitro, hydroxyl, mercapto, amino, carboxyl, alkyl, haloalkyl, alkenyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (—N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms;
R¹ is C₁-C₁₂-haloalkyl, C₂-C₁₂-haloalkenyl, or C₂-C₁₂-haloalkynyl;
R² is H, R¹, C₁-C₁₂-alkyl, C₂-C₁₂-alkenyl, C₂-C₁₂-alkynyl, C₃-C₈-cycloalkyl, C₃-C₈-halocycloalkyl, C₃-C₆-cycloalkenyl, C₃-C₆-halocycloalkenyl, C₁-C₈-alkoxy, C₂-C₈-alkenyloxy, C₂-C₈-alkynyloxy, C₃-C₈-cycloalkoxy, NH₂, C₁-C₈-alkylamino, di-C₁-C₈-alkylamino, phenyl, naphthyl or a five- or six-membered saturated, partially unsaturated or aromatic heterocycle comprising one, two, three or four heteroatoms selected from the group consisting of O, N and S, or Z—Y—(CR⁷R⁸)ₚ—(CR⁵R⁶)_{q}—CR³R⁴—# where # is the bonding site to the nitrogen atom and:
R³, R⁴, R⁵, R⁶, R⁷, R⁸ are each independently hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl, C₃-C₆-cycloalkenyl, C₃-C₆-halocycloalkenyl, phenyl, naphthyl or a five- or six-membered saturated, partially unsaturated or aromatic heterocycle comprising one, two, three or four heteroatoms selected from the group consisting of O, N and S,
R⁵ may also, with R³ or R⁷, together with the atoms to which these radicals are bonded, form a five-, six-, seven-, eight-, nine- or ten-membered saturated or partially unsaturated ring which, as well as carbon atoms, may comprise one, two or three heteroatoms selected from the group consisting of O, N and S as ring members and/or may bear one or more substituents R^{a};
R³ with R⁴, R⁵ with R⁶, R⁷ with R⁸ may each together also form carbonyl groups by being oxygen and form spiro groups by forming a C₂-C₈-alkylene or alkenylene, alkynylene chain which may be interrupted by one, two or three heteroatoms selected from the group consisting of O, N and S;
R¹ to R⁸ may each independently bear one, two, three or four identical or different R^{a} groups;
Y is oxygen or sulfur;
Z is hydrogen, carboxyl, formyl, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, C(NR^{A})SR^{Π}, C(S)R^{Π}, C(NR^{Π})NR^{A}R^{B}, C(NR^{Π})R^{A}, C(NR^{Π})OR^{A}, C(O)NR^{A}R^{B}, C(S)NR^{A}R^{B}, C₁-C₈-alkylsulfinyl, C₁-C₈-alkylthio, C₁-C₈-alkylsulfonyl, C(O)—C₁-C₄-alkyleneNR^{A}C(NR^{Π})NR^{A}R^{B},
C(S)—C₁-C₄-alkylene-NR^{A}C(NR^{Π})NR^{A}R^{B},
C(NR¹)—C₁-C₄-alkylene-NR^{A}C(NR^{Π})NR^{A}R^{B}, phenyl, naphthyl, five-, six-, seven-, eight-, nine- or ten-membered saturated, partially unsaturated or aromatic heterocycle which comprises one, two, three or four heteroatoms selected from the group consisting of O, N and S and is bonded directly or via a carbonyl, thiocarbonyl, C₁-C₄-alkylcarbonyl or C₁-C₄-alkylthiocarbonyl group; where the carbon chains in the Z group may be substituted by one or more R^{b} groups;
R^{A}, R^{B} are each independently hydrogen, C₂-alkenyl, C₂-alkynyl or one of the groups mentioned for R^{Π};
R^{A} and R^{B}, together with the nitrogen atom to which they are bonded, or R^{A} and R^{Π} together with the carbon atoms and heteroatoms via which they are bonded, may also form a five- or six-membered saturated, partly unsaturated or aromatic ring which, as well as carbon atoms, may comprise one, two or three further heteroatoms selected from the group consisting of O, N and S as ring members and/or may bear one or more substituents R^{a};
or
Z may also, with R⁶ or R⁸, form a five- or six-membered saturated or partly unsaturated ring which, as well as carbon atoms and Y, may comprise one or two further heteroatoms selected from the group consisting of N and S as ring members and/or may bear one or more substituents R^{a} as defined below;
the Z group may be partially or completely halogenated and/or bear one, two or three R^{b} groups;
W is phenyl or five- or six-membered heteroaryl which, as well as carbon atoms, comprises one, two or three further heteroatoms selected from the group consisting of O, N and S as ring members, where the ring systems, as well as Lₘ groups, bear at least one substituent P¹,
P¹ is Y¹—Y²-T;
Y¹ is CR^{a}R^{a′}, C(O)O, C(O)NR^{b}, O, NR or S(O)ᵣ;
Y² is C₃-C₈-alkylene, C₂-C₈-alkenylene, C₂-C₈-alkynylene, C₃-C₈-alkylene-(Y³—(C₂-C₄-alkylene))ₛ, C₃-C₈-alkenylene-(Y³—(C₂-C₄-alkylene))ₛ, C₃-C₈-alkynylene-(Y³—(C₂-C₄-alkylene))ₛ, and, if R is C₃-C₆-cycloalkyl or C₃-C₁₂-halocycloalkyl and/or Y¹ is CR^{a}R^{a′}, C(O)O, CONR^{b}, NR^{b} or S(O)ᵣ, is also C₁-C₂-alkylene, and/or, if T is OC(O)R^{a}, NR^{b}R^{b′}, C(O)NR^{b}R^{b′}, C(NOR^{c})R^{a} or T¹-C(=T²)-T³ is also C₁-C₂-alkylene;
Y³ is O, S, or NR^{b}
r is 0, 1 or 2
s 1 or 2,
T is OR^{c}, wherein R^{c} is other than H, OC(O)R^{a}, C(O)NR^{b}R^{b′}, C(NOR^{c})R^{a}, T¹-C(=T²)-T³, and, if R is defined as NR¹R² or C₃-C₁₂-halocycloalkyl, is also C(O)OR^{c} or C(OR^{c})₂R^{d},
T¹ is O, or NR^{b};
T² is O, S, or NR^{b};
T³ is R^{a}, OR^{c}, SR^{c}, or NR^{b}R^{b′},
where R^{a′} and R^{b′}, independently of R^{a} and R^{b} respectively, are each as defined for these radicals;
L is halogen, hydroxyl, cyanato (OCN), cyano, nitro, C₁-C₈-alkyl, C₁-C₈-haloalkyl, C₂-C₁₀-alkenyl, C₂-C₁₀-haloalkenyl, C₂-C₁₀-alkynyl, C₃-C₆-cycloalkyl, C₃-C₆-halocycloalkyl, C₃-C₆-cycloalkenyl, C₁-C₈-alkoxy, C₁-C₈-haloalkoxy, C₂-C₁₀-alkenyloxy, C₂-C₁₀-alkynyloxy, C₃-C₆-cycloalkyloxy, C₃-C₆-cycloalkenyloxy, amino, C₁-C₄-alkylamino, di-(C₁-C₄)-alkylamino, C₁-C₄-alkylcarbonylamino, C(O)—R^{Φ}, C(S)—R^{Φ}, S(O)ₙ—R^{Φ}; C₁-C₈-alkoxyimino-(C₁-C₈)-alkyl, C₂-C₁₀-alkenyloxyimino-(C₁-C₈)-alkyl, C₂-C₁₀-alkynyloxyimino-(C₁-C₈)-alkyl, C₂-C₁₀-alkynylcarbonyl, C₃-C₆-cycloalkylcarbonyl, or a five-, six-, seven-, eight-, nine- or ten-membered saturated, partly unsaturated or aromatic heterocycle comprising one, two, three or four heteroatoms selected from the group consisting of O, N and S;
R^{Φ} is hydrogen, C₁-C₄-alkyl, C₁-C₂-haloalkyl, C₁-C₄-alkoxy, C₂-C₄-alkenyloxy, C₂-C₄-alkynyloxy, amino, C₁-C₄-alkylamino, or di-C₁-C₄-alkylamino; where the R¹ groups may be substituted by one, two or three identical or different R^{b} groups as defined above;
n is zero, 1 or 2;
m is zero, 1, 2, 3, 4 or 5 and
X is halogen, cyano, C₁-C₄-alkyl, C₁-C₄-haloalkyl, C₁-C₄-alkoxy or C₁-C₄-haloalkoxy.

39: A compound of formula I 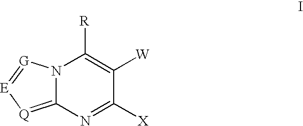
or an agriculturally acceptable salt thereof, wherein the substituents in the formula (I) are each defined as follows:
G, E, Q a)G is N; E is C—W² and Q is N or C—W³;
b)G is C—W¹; E is C—W² and Q is N; or
c) G is C—W¹; E is N and Q is C—W³;
W¹, W², W³ are each independently selected from the group consisting of hydrogen, halogen, cyano, nitro, C₁-C₄-alkyl, C₂-C₆-alkenyl, C₂-C₆-alkynyl, C₁-C₄-haloalkyl, hydroxy-C₁-C₄-alkyl, C₁-C₄-alkoxy-C₁-C₄-alkyl, C₂-C₆-haloalkenyl, C₂-C₆-haloalkynyl, C₃-C₆-cycloalkyl, C₃-C₆-halocycloalkyl, C₁-C₄-alkoxy, C₁-C₄-haloalkoxy, C₁-C₄-alkylthio, C₁-C₄-alkylsulfinyl, C₁-C₄-alkylsulfonyl, formyl, thiocarbamoyl, C₁-C₄-alkylcarbonyl, C₁-C₄-alkoxycarbonyl, C₁-C₄-alkylaminocarbonyl, aminocarbonyl, di(C₁-C₄-alkyl)aminocarbonyl, C₁-C₄-alkoximinoalkyl, hydroximinoalkyl, and CR¹⁰R¹¹OR¹², C(R¹³)═NR¹⁴;
R¹⁰, R¹¹, R¹² are each independently hydrogen, C₁-C₈-alkyl, C₃-C₆-cycloalkyl, C₁-C₈-alkoxy-C₁-C₈-alkyl, C₂-C₈-alkenyl, C₂-C₈-alkynyl, or benzyl;
R¹¹ and R¹² may together be oxy-C₁-C₅-alkyleneoxy in which the carbon chain may be substituted by from one to three groups selected from the group consisting of methyl, ethyl, hydroxyl, methoxy, ethoxy, hydroxymethyl, methoxymethyl, and ethoxymethyl;
R¹³ is hydrogen or C₁-C₈-alkyl;
R¹⁴ is C₁-C₈-alkyl, C₃-C₆-cycloalkyl, phenyl, or phenylamino, where the phenyl groups may be substituted by from one to five R^{b} groups;
R is NR¹R², C₃-C₆-cycloalkyl or C₃-C₁₂-halocycloalkyl; where R may comprise one, two, three or four identical or different R^{a} groups which are each independently selected from:
R^{a} is R^{b}, carboxyl, OC(O)OR^{Π} or C₁-C₆-alkylthio;
R^{b} is R^{c}, hydroxyl, C₁-C₆-alkoxy, C₂-C₆-alkenyloxy, C₃-C₆-alkynyloxy, C₃-C₆-cycloalkoxy or C₃-C₆-cycloalkenyloxy;
R^{c} is hydrogen, cyano, C₁-C₆-alkyl, C₂-C₆-alkenyl, C₂-C₆-alkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, amino, C₁-C₆-alkylamino, di-C₁-C₆-alkylamino, aminocarbonyl, C(O)NHR^{Π}, C(O)NR^{Π}₂, phenyl, naphthyl, five-, six-, seven-, eight-, nine- or ten-membered saturated, partially unsaturated or aromatic heterocycle comprising one, two, three or four heteroatoms selected from the group consisting of O, N and S,
or two R^{a}, R^{b}, R^{C}, R^{Π}, R^{d} and/or R^{dd} groups form, together with the atom or the atoms to which they are bonded, a 3- to 12-membered saturated, partially unsaturated or aromatic ring which is carbocyclic or comprises from one to four heteroatoms selected from the group consisting of N, O and S and which is unsubstituted or substituted by from 1 to 4 R^{dd} radicals, in the case of halogen even up to the maximum number;
R^{Π} is C₁-C₈-alkyl, C₃-C₈-alkenyl, C₃-C₈-alkynyl, C₃-C₆-cycloalkyl or C₃-C₆-cycloalkenyl;
where the aliphatic, alicyclic or aromatic groups in the aforementioned R^{a}, R^{b}, R^{c} and R^{Π} groups may in turn be partially or fully halogenated and/or bear one, two or three R^{d} groups:
R^{d} is halogen, cyano, nitro, hydroxyl, mercapto, amino, carboxyl, alkyl, haloalkyl, alkenyl, alkynyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms; cycloalkyl, cycloalkoxy, heterocyclyl, heterocyclyloxy, where the cyclic systems comprise from 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals comprise preferably from 6 to 10 ring members, the hetaryl radicals 5 or 6 ring members, where the cyclic systems are partially or completely halogenated and/or may bear from 1 to 3 R^{dd} groups;
R^{dd} is halogen, cyano, nitro, hydroxyl, mercapto, amino, carboxyl, alkyl, haloalkyl, alkenyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms;
R¹ is C₁-C₁₂-haloalkyl, C₂-C₁₂-haloalkenyl, or C₂-C₁₂-haloalkynyl;
R² is H, R¹, C₁-C₁₂-alkyl, C₂-C₁₂-alkenyl, C₂-C₁₂-alkynyl, C₃-C₈-cycloalkyl, C₃-C₈-halocycloalkyl, C₃-C₆-cycloalkenyl, C₃-C₆-halocycloalkenyl, C₁-C₈-alkoxy, C₂-C₈-alkenyloxy, C₂-C₈-alkynyloxy, C₃-C₈-cycloalkoxy, NH₂, C₁-C₈-alkylamino, di-C₁-C₈-alkylamino, phenyl, naphthyl or a five- or six-membered saturated, partially unsaturated or aromatic heterocycle comprising one, two, three or four heteroatoms selected from the group consisting of O, N and S, or Z—Y—(CR⁷R⁸)ₚ—(CR⁵R⁶)_{q}—CR³R⁴—# where # is the bonding site to the nitrogen atom and:
R³, R⁴, R⁵, R⁶, R⁷, R⁸ are each independently hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl, C₃-C₆-cycloalkenyl, C₃-C₆-halocycloalkenyl, phenyl, naphthyl or a five- or six-membered saturated, partially unsaturated or aromatic heterocycle comprising one, two, three or four heteroatoms selected from the group consisting of O, N and S,
R⁵ may also, with R³ or R⁷, together with the atoms to which these radicals are bonded, form a five-, six-, seven-, eight-, nine- or ten-membered saturated or partially unsaturated ring which, as well as carbon atoms, may comprise one, two or three heteroatoms selected from the group consisting of O, N and S as ring members and/or may bear one or more substituents R^{a};
R³ with R⁴, R⁵ with R⁶, R⁷ with R⁸ may each together also form carbonyl groups by being oxygen and form spiro groups by forming a C₂-C₅-alkylene or alkenylene, alkynylene chain which may be interrupted by one, two or three heteroatoms selected from the group consisting of O, N and S;
R¹ to R⁸ may each independently bear one, two, three or four identical or different R^{a} groups;
Y is oxygen or sulfur;
Z is hydrogen, carboxyl, formyl, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, C(NR^{A})SR^{Π}, C(S)R^{Π}, C(NR^{Π})NR^{A}R^{B}, C(NR^{Π})R^{A}, C(NR^{Π})OR^{A}, C(O)NR^{A}R^{B}, C(S)NR^{A}R^{B}, C₁-C₈-alkylsulfinyl, C₁-C₈-alkylthio, C₁-C₈-alkylsulfonyl, C(O)—C₁-C₄-alkyleneNR^{A}C(NR^{Π}) NR^{A}R^{B},
C(S)—C₁-C₄-alkylene-NR^{A}C(NR^{Π})NR^{A}R^{B},
C(NR^{Π})—C₁-C₄-alkylene-NR^{A}C(NR^{Π})NR^{A}R^{B}, phenyl, naphthyl, five-, six-, seven-, eight-, nine- or ten-membered saturated, partially unsaturated or aromatic heterocycle which comprises one, two, three or four heteroatoms selected from the group consisting of O, N and S and is bonded directly or via a carbonyl, thiocarbonyl, C₁-C₄-alkylcarbonyl or C₁-C₄-alkylthiocarbonyl group; where the carbon chains in the Z group may be substituted by one or more R^{b} groups;
R^{A}, R^{B} are each independently hydrogen, C₂-alkenyl, C₂-alkynyl or one of the groups mentioned for R^{Π};
R^{A} and R^{B}, together with the nitrogen atom to which they are bonded, or R^{A} and R^{Π} together with the carbon atoms and heteroatoms via which they are bonded, may also form a five- or six-membered saturated, partly unsaturated or aromatic ring which, as well as carbon atoms, may comprise one, two or three further heteroatoms selected from the group consisting of O, N and S as ring members and/or may bear one or more substituents R^{a};
or
Z may also, with R⁶ or R⁸, form a five- or six-membered saturated or partly unsaturated ring which, as well as carbon atoms and Y, may comprise one or two further heteroatoms selected from the group consisting of N and S as ring members and/or may bear one or more substituents R^{a} as defined below;
the Z group may be partially or completely halogenated and/or bear one, two or three R^{b} groups;
W is five- or six-membered heteroaryl which, as well as carbon atoms, comprises one, two or three further heteroatoms from the group of O, N and S as ring members, where the ring systems, as well as Lₘ groups, bear at least one substituent P¹,
P¹ is Y¹—Y²-T;
Y¹ is CR^{a}R^{a′}, C(O)O, C(O)NR^{b}, O, NR^{b} or S(O)ᵣ;
Y² is C₃-C₈-alkylene, C₂-C₈-alkenylene, C₂-C₈-alkynylene, C₃-C₈-alkylene-(Y³—(C₂-C₄-alkylene))ₛ, C₃-C₈-alkenylene-(Y³—(C₂-C₄-alkylene))ₛ, C₃-C₈-alkynylene-(Y³—(C₂-C₄-alkylene))ₛ, and, if R is C₃-C₆-cycloalkyl or C₃-C₁₂-halocycloalkyl and/or Y¹ is CR^{a}R^{a′}, C(O)O, CONR^{b}, NR^{b} or S(O)ᵣ, is also C₁-C₂-alkylene, and/or, if T is OC(O)R^{a}, NR^{b}R^{b′}, C(O)NR^{b}R^{b′}, C(NOR^{c})R^{a} or T¹-C(=T²)-T³, is also C₁-C₂-alkylene;
Y³ is O, S, or NR^{b}
r is 0, 1 or 2
s 1 or 2,
T is OR^{c}, OC(O)R^{a}, NR^{b}R^{b′}, C(O)NR^{b}R^{b′}, C(NOR^{c})R^{a}, or T¹-C(=T²)-T³ and, if R is defined as NR¹R² or C₃-C₁₂-halocycloalkyl, is also C(O)OR^{c} or C(OR^{c})₂R^{a};
T¹ is O, or NR^{b};
T² is O, S, or NR^{b};
T³ is R^{a}, OR^{c}, SR^{c}, or NR^{b}R^{b′},
where R^{a′} and R^{b′}, independently of R^{a} and R^{b} respectively, are each as defined for these radicals;
L is halogen, hydroxyl, cyanato (OCN), cyano, nitro, C₁-C₈-alkyl, C₁-C₈-haloalkyl, C₂-C₁₀-alkenyl, C₂-C₁₀-haloalkenyl, C₂-C₁₀-alkynyl, C₃-C₆-cycloalkyl, C₃-C₆-halocycloalkyl, C₃-C₆-cycloalkenyl, C₁-C₈-alkoxy, C₁-C₈-haloalkoxy, C₂-C₁₀-alkenyloxy, C₂-C₁₀-alkynyloxy, C₃-C₆-cycloalkyloxy, C₃-C₆-cycloalkenyloxy, amino, C₁-C₄-alkylamino, di-(C₁-C₄)-alkylamino, C₁-C₄-alkylcarbonylamino, C(O)—R^{Φ}, C(S)—R^{e}, S(O)ₙ—R^{Φ}; C₁-C₈-alkoxyimino-(C₁-C₈)-alkyl, C₂-C₁₀-alkenyloxyimino-(C₁-C₈)-alkyl, C₂-C₁₀-alkynyloxyimino-(C₁-C₈)-alkyl, C₂-C₁₀-alkynylcarbonyl, C₃-C₆-cycloalkylcarbonyl, or a five-, six-, seven-, eight-, nine- or ten-membered saturated, partly unsaturated or aromatic heterocycle comprising one, two, three or four heteroatoms selected from the group consisting of O, N and S;
R^{Φ} is hydrogen, C₁-C₄-alkyl, C₁-C₂-haloalkyl, C₁-C₄-alkoxy, C₂-C₄-alkenyloxy, C₂-C₄-alkynyloxy, amino, C₁-C₄-alkylamino, or di-C₁-C₄-alkylamino; where the R¹ groups may be substituted by one, two or three identical or different R^{b} groups as defined above;
n is zero, 1 or 2;
m is zero, 1, 2, 3, 4 or 5 and
X is halogen, cyano, C₁-C₄-alkyl, C₁-C₄-haloalkyl, C₁-C₄-alkoxy or C₁-C₄-haloalkoxy.

40: A compound of formula I 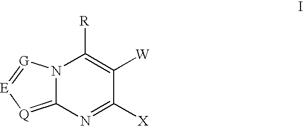
or an agriculturally acceptable salt thereof, wherein the substituents in the formula (I) are each defined as follows:
G, E, Q G is C—W¹; E is N and Q is C—W³;
W¹, W², W³ are each independently selected from the group consisting of hydrogen, halogen, cyano, nitro, C₁-C₄-alkyl, C₂-C₆-alkenyl, C₂-C₆-alkynyl, C₁-C₄-haloalkyl, hydroxy-C₁-C₄-alkyl, C₁-C₄-alkoxy-C₁-C₄-alkyl, C₂-C₆-haloalkenyl, C₂-C₆-haloalkynyl, C₃-C₆-cycloalkyl, C₃-C₆-halocycloalkyl, C₁-C₄-alkoxy, C₁-C₄-haloalkoxy, C₁-C₄-alkylthio, C₁-C₄-alkylsulfinyl, C₁-C₄-alkylsulfonyl, formyl, thiocarbamoyl, C₁-C₄-alkylcarbonyl, C₁-C₄-alkoxycarbonyl, C₁-C₄-alkylaminocarbonyl, aminocarbonyl, di(C₁-C₄-alkyl)aminocarbonyl, C₁-C₄-alkoximinoalkyl, hydroximinoalkyl, and CR¹⁰R¹¹R¹², C(R¹³)═NR¹⁴;
R¹⁰, R¹¹, R¹² are each independently hydrogen, C₁-C₈-alkyl, C₃-C₆-cycloalkyl, C₁-C₈-alkoxy-C₁-C₈-alkyl, C₂-C₈-alkenyl, C₂-C₈-alkynyl, or benzyl;
R¹¹ and R¹² may together be oxy-C₁-C₅-alkyleneoxy in which the carbon chain may be substituted by from one to three groups selected from the group consisting of methyl, ethyl, hydroxyl, methoxy, ethoxy, hydroxymethyl, methoxymethyl, and ethoxymethyl;
R¹³ is hydrogen or C₁-C₈-alkyl;
R¹⁴ is C₁-C₈-alkyl, C₃-C₆-cycloalkyl, phenyl, or phenylamino, where the phenyl groups may be substituted by from one to five R^{b} groups;
R is NR¹R², C₃-C₆-cycloalkyl or C₃-C₁₂-halocycloalkyl; where R^{a} may comprise one, two, three or four identical or different R^{a} groups which are each independently selected from:
R^{a} is R^{b}, carboxyl, OC(O)OR^{Π} or C₁-C₆-alkylthio;
R^{b} is R^{c}, hydroxyl, C₁-C₆-alkoxy, C₂-C₆-alkenyloxy, C₃-C₆-alkynyloxy, C₃-C₆-cycloalkoxy or C₃-C₆-cycloalkenyloxy;
R^{c} is hydrogen, cyano, C₁-C₆-alkyl, C₂-C₆-alkenyl, C₂-C₆-alkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, amino, C₁-C₆-alkylamino, di-C₁-C₆-alkylamino, aminocarbonyl, C(O)NHR^{Π}, C(O)NR^{Π}₂, phenyl, naphthyl, five-, six-, seven-, eight-, nine- or ten-membered saturated, partially unsaturated or aromatic heterocycle comprising one, two, three or four heteroatoms selected from the group consisting of O, N and S,
or two R^{a}, R^{b}, R^{C}, R^{Π}, R^{d} and/or R^{dD} groups form, together with the atom or the atoms to which they are bonded, a 3- to 12-membered saturated, partially unsaturated or aromatic ring which is carbocyclic or comprises from one to four heteroatoms selected from the group consisting of N, O and S and which is unsubstituted or substituted by from 1 to 4 R^{d} radicals, in the case of halogen even up to the maximum number;
R^{Π} is C₁-C₈-alkyl, C₃-C₈-alkenyl, C₃-C₈-alkynyl, C₃-C₆-cycloalkyl or C₃-C₆-cycloalkenyl;
where the aliphatic, alicyclic or aromatic groups in the aforementioned R^{a}, R^{b}, R^{c} and R^{Π} groups may in turn be partially or fully halogenated and/or bear one, two or three R^{d} groups:
R^{d} is halogen, cyano, nitro, hydroxyl, mercapto, amino, carboxyl, alkyl, haloalkyl, alkenyl, alkynyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms; cycloalkyl, cycloalkoxy, heterocyclyl, heterocyclyloxy, where the cyclic systems comprise from 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals comprise preferably from 6 to 10 ring members, the hetaryl radicals 5 or 6 ring members, where the cyclic systems are partially or completely halogenated and/or may bear from 1 to 3 R^{dd} groups;
R^{dd} is halogen, cyano, nitro, hydroxyl, mercapto, amino, carboxyl, alkyl, haloalkyl, alkenyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms;
R¹ is C₁-C₁₂-haloalkyl, C₂-C₁₂-haloalkenyl, or C₂-C₁₂-haloalkynyl;
R² is H, R¹, C₁-C₁₂-alkyl, C₂-C₁₂-alkenyl, C₂-C₁₂-alkynyl, C₃-C₈-cycloalkyl, C₃-C₈-halocycloalkyl, C₃-C₆-cycloalkenyl, C₃-C₆-halocycloalkenyl, C₁-C₈-alkoxy, C₂-C₈-alkenyloxy, C₂-C₈-alkynyloxy, C₃-C₈-cycloalkoxy, NH₂, C₁-C₈-alkylamino, di-C₁-C₈-alkylamino, phenyl, naphthyl or a five- or six-membered saturated, partially unsaturated or aromatic heterocycle comprising one, two, three or four heteroatoms selected from the group consisting of O, N and S, or Z—Y—(CR⁷R⁸)ₚ—(CR⁵R⁶)_{q}—CR³R⁴—# where # is the bonding site to the nitrogen atom and:
R³, R⁴, R⁵, R⁶, R⁷, R⁸ are each independently hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl, C₃-C₆-cycloalkenyl, C₃-C₆-halocycloalkenyl, phenyl, naphthyl or a five- or six-membered saturated, partially unsaturated or aromatic heterocycle comprising one, two, three or four heteroatoms selected from the group consisting of O, N and S,
R⁵ may also, with R³ or R⁷, together with the atoms to which these radicals are bonded, form a five-, six-, seven-, eight-, nine- or ten-membered saturated or partially unsaturated ring which, as well as carbon atoms, may comprise one, two or three heteroatoms selected from the group consisting of O, N and S as ring members and/or may bear one or more substituents R^{a};
R³ with R⁴, R⁵ with R⁶, R⁷ with R⁸ may each together also form carbonyl groups by being oxygen and form spiro groups by forming a C₂-C₅-alkylene or alkenylene, alkynylene chain which may be interrupted by one, two or three heteroatoms selected from the group consisting of O, N and S;
R¹ to R⁸ may each independently bear one, two, three or four identical or different R^{a} groups;
Y is oxygen or sulfur;
Z is hydrogen, carboxyl, formyl, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, C(NR^{A})SR^{Π}, C(S)R^{Π}, C(NR^{Π})NR^{A}R^{B}, C(NR^{Π})R^{A}, C(NR^{Π})OR^{A}, C(O)NR^{A}R^{B}, C(S)NR^{A}R^{B}, C₁-C₈-alkylsulfinyl, C₁-C₈-alkylthio, C₁-C₈-alkylsulfonyl, C(O)—C₁-C₄-alkyleneNR^{A}C(NR^{Π})NR^{A}R^{B},
C(S)—C₁-C₄-alkylene-NR^{A}C(NR^{Π})NR^{A}R^{B},
C(NR^{Π})-C₁-C₄-alkylene-NR^{A}C(NR^{Π})NR^{A}R^{B}, phenyl, naphthyl, five-, six-, seven-, eight-, nine- or ten-membered saturated, partially unsaturated or aromatic heterocycle which comprises one, two, three or four heteroatoms selected from the group consisting of O, N and S and is bonded directly or via a carbonyl, thiocarbonyl, C₁-C₄-alkylcarbonyl or C₁-C₄-alkylthiocarbonyl group; where the carbon chains in the Z group may be substituted by one or more R^{b} groups;
R^{A}, R^{B} are each independently hydrogen, C₂-alkenyl, C₂-alkynyl or one of the groups mentioned for R^{Π};
R^{A} and R^{B}, together with the nitrogen atom to which they are bonded, or R^{A} and R^{Π} together with the carbon atoms and heteroatoms via which they are bonded, may also form a five- or six-membered saturated, partly unsaturated or aromatic ring which, as well as carbon atoms, may comprise one, two or three further heteroatoms selected from the group consisting of O, N and S as ring members and/or may bear one or more substituents R^{a};
or
Z may also, with R⁶ or R⁸, form a five- or six-membered saturated or partly unsaturated ring which, as well as carbon atoms and Y, may comprise one or two further heteroatoms selected from the group consisting of N and S as ring members and/or may bear one or more substituents R^{a} as defined below;
the Z group may be partially or completely halogenated and/or bear one, two or three R^{b} groups;
W is phenyl or five- or six-membered heteroaryl which, as well as carbon atoms, comprises one, two or three further heteroatoms selected from the group consisting of O, N and S as ring members, where the ring systems, as well as Lₘ groups, bear at least one substituent P¹,
P¹ is Y¹—Y²-T;
Y¹ is CR^{a}R^{a′}, C(O)O, C(O)NR^{b}, O, NR^{b} or S(O)ᵣ;
Y² is C₃-C₈-alkylene, C₂-C₈-alkenylene, C₂-C₈-alkynylene, C₃-C₈-alkylene-(Y³—(C₂-C₄-alkylene))ₛ^{, C}₃-C₈-alkenylene-(Y³—(C₂-C₄-alkylene))ₛ, C₃-C₈-alkynylene-(Y³—(C₂-C₄-alkylene))ₛ, and, if R is C₃-C₆-cycloalkyl or C₃-C₁₂-halocycloalkyl and/or Y¹ is CR^{a}R^{a′}, C(O)O, CONR^{b}, NR^{b} or S(O)ᵣ, is also C₁-C₂-alkylene, and/or, if T is OC(O)R^{a}, NR^{b}R^{b′}, C(O)NR^{b}R^{b′}, C(NOR^{c})R^{a} or T¹-C(=T²)-T³, is also C₁-C₂-alkylene;
Y³ is O, S, or NR^{b}
r is 0, 1 or 2
s 1 or 2,
T is OR^{c}, OC(O)R^{a}, NR^{b}R^{b′}, C(O)NR^{b}R^{b′}, C(NOR^{c})R^{a}, or T¹-C(=T²)-T³, and, if R is defined as NR¹R² or C₃-C₁₂-halocycloalkyl, is also C(O)OR^{c} or C(OR^{c})₂R^{a};
T¹ is O, or NR^{b};
T² is O, S, or NR^{b};
T³ is R^{a}, OR^{c}, SR^{c}, or NR^{b}R^{b′},
where R^{a′} and R^{b′}, independently of R^{a} and R^{b} respectively, are each as defined for these radicals;
L is halogen, hydroxyl, cyanato (OCN), cyano, nitro, C₁-C₈-alkyl, C₁-C₈-haloalkyl, C₂-C₁₀-alkenyl, C₂-C₁₀-haloalkenyl, C₂-C₁₀-alkynyl, C₃-C₆-cycloalkyl, C₃-C₆-halocycloalkyl, C₃-C₆-cycloalkenyl, C₁-C₈-alkoxy, C₁-C₈-haloalkoxy, C₂-C₁₀-alkenyloxy, C₂-C₁₀-alkynyloxy, C₃-C₆-cycloalkyloxy, C₃-C₆-cycloalkenyloxy, amino, C₁-C₄-alkylamino, di-(C₁-C₄)-alkylamino, C₁-C₄-alkylcarbonylamino, C(O)—R^{Φ}, C(S)—R^{Φ}, S(O)ₙ—R^{Φ}; C₁-C₈-alkoxyimino-(C₁-C₈)-alkyl, C₂-C₁₀-alkenyloxyimino-(C₁-C₈)-alkyl, C₂-C₁₀-alkynyloxyimino-(C₁-C₈)-alkyl, C₂-C₁₀-alkynylcarbonyl, C₃-C₆-cycloalkylcarbonyl, or a five-, six-, seven-, eight-, nine- or ten-membered saturated, partly unsaturated or aromatic heterocycle comprising one, two, three or four heteroatoms selected from the group consisting of O, N and S;
R^{Φ} is hydrogen, C₁-C₄-alkyl, C₁-C₂-haloalkyl, C₁-C₄-alkoxy, C₂-C₄-alkenyloxy, C₂-C₄-alkynyloxy, amino, C₁-C₄-alkylamino, or di-C₁-C₄-alkylamino;
where the R^{Φ} groups may be substituted by one, two or three identical or different R^{b} groups as defined above;
n is zero, 1 or 2;
m is zero, 1, 2, 3, 4 or 5 and
X is halogen, cyano, C₁-C₄-alkyl, C₁-C₄-haloalkyl, C₁-C₄-alkoxy or C₁-C₄-haloalkoxy.

41: A compound of formula I 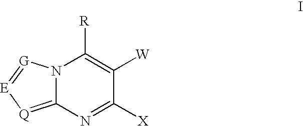
or an agriculturally acceptable salt thereof, wherein the substituents in the formula (I) are each defined as follows:
G, E, Q a) G is N; E is C—W² and Q is N or C—W³;
b) G is C—W¹; E is C—W² and Q is N; or
c) G is C—W¹; E is N and Q is C—W³;
W¹, W², W³ are each independently selected from the group consisting of hydrogen, halogen, cyano, nitro, C₁-C₄-alkyl, C₂-C₆-alkenyl, C₂-C₆-alkynyl, C₁-C₄-haloalkyl, hydroxy-C₁-C₄-alkyl, C₁-C₄-alkoxy-C₁-C₄-alkyl, C₂-C₆-haloalkenyl, C₂-C₆-haloalkynyl, C₃-C₆-cycloalkyl, C₃-C₆-halocycloalkyl, C₁-C₄-alkoxy, C₁-C₄-haloalkoxy, C₁-C₄-alkylthio, C₁-C₄-alkylsulfinyl, C₁-C₄-alkylsulfonyl, formyl, thiocarbamoyl, C₁-C₄-alkylcarbonyl, C₁-C₄-alkoxycarbonyl, C₁-C₄-alkylaminocarbonyl, aminocarbonyl, di(C₁-C₄-alkyl)aminocarbonyl, C₁-C₄-alkoximinoalkyl, hydroximinoalkyl, and CR¹⁰R¹¹OR², C(R³)═NR¹⁴;
R¹⁰, R¹¹, R¹² are each independently hydrogen, C₁-C₈-alkyl, C₃-C₆-cycloalkyl, C₁-C₈-alkoxy-C₁-C₈-alkyl, C₂-C₈-alkenyl, C₂-C₈-alkynyl, or benzyl;
R¹¹ and R¹² may together be oxy-C₁-C₅-alkyleneoxy in which the carbon chain may be substituted by from one to three groups selected from the group consisting of methyl, ethyl, hydroxyl, methoxy, ethoxy, hydroxymethyl, methoxymethyl, and ethoxymethyl;
R¹³ is hydrogen or C₁-C₈-alkyl;
R¹⁴ is C₁-C₈-alkyl, C₃-C₆-cycloalkyl, phenyl, or phenylamino, where the phenyl groups may be substituted by from one to five R^{b} groups;
R is NR¹R², C₃-C₆-cycloalkyl or C₃-C₁₂-halocycloalkyl; where R may comprise one, two, three or four identical or different R^{a} groups which are each independently selected from:
R^{a} is R^{b}, carboxyl, OC(O)OR^{Π} or C₁-C₆-alkylthio;
R^{b} is R^{c}, hydroxyl, C₁-C₆-alkoxy, C₂-C₆-alkenyloxy, C₃-C₆-alkynyloxy, C₃-C₆-cycloalkoxy or C₃-C₆-cycloalkenyloxy;
R^{c} is hydrogen, cyano, C₁-C₆-alkyl, C₂-C₆-alkenyl, C₂-C₆-alkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, amino, C₁-C₆-alkylamino, di-C₁-C₆-alkylamino, aminocarbonyl, C(O)NHR^{Π}, C(O)NR^{Π}₂, phenyl, naphthyl, five-, six-, seven-, eight-, nine- or ten-membered saturated, partially unsaturated or aromatic heterocycle comprising one, two, three or four heteroatoms selected from the group consisting of O, N and S,
or two R^{a}, R^{b}, R^{C}, R^{Π}, R^{d} and/or R^{dd} groups form, together with the atom or the atoms to which they are bonded, a 3- to 12-membered saturated, partially unsaturated or aromatic ring which is carbocyclic or comprises from one to four heteroatoms selected from the group consisting of N, O and S and which is unsubstituted or substituted by from 1 to 4 R^{d} radicals, in the case of halogen even up to the maximum number;
R^{Π} is C₁-C₈-alkyl, C₃-C₈-alkenyl, C₃-C₈-alkynyl, C₃-C₆-cycloalkyl or C₃-C₆-cycloalkenyl;
where the aliphatic, alicyclic or aromatic groups in the aforementioned R^{a}, R^{b}, R^{c} and R^{Π} groups may in turn be partially or fully halogenated and/or bear one, two or three R^{d} groups:
R^{d} is halogen, cyano, nitro, hydroxyl, mercapto, amino, carboxyl, alkyl, haloalkyl, alkenyl, alkynyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms; cycloalkyl, cycloalkoxy, heterocyclyl, heterocyclyloxy, where the cyclic systems comprise from 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals comprise preferably from 6 to 10 ring members, the hetaryl radicals 5 or 6 ring members, where the cyclic systems are partially or completely halogenated and/or may bear from 1 to 3 R^{dd} groups;
R^{dd} is halogen, cyano, nitro, hydroxyl, mercapto, amino, carboxyl, alkyl, haloalkyl, alkenyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms;
R¹ is C₁-C₁₂-haloalkyl, C₂-C₁₂-haloalkenyl, or C₂-C₁₂-haloalkynyl;
R² is H, R¹, C₁-C₁₂-alkyl, C₂-C₁₂-alkenyl, C₂-C₁₂-alkynyl, C₃-C₈-cycloalkyl, C₃-C₈-halocycloalkyl, C₃-C₆-cycloalkenyl, C₃-C₆-halocycloalkenyl, C₁-C₈-alkoxy, C₂-C₈-alkenyloxy, C₂-C₈-alkynyloxy, C₃-C₈-cycloalkoxy, NH₂, C₁-C₈-alkylamino, di-C₁-C₈-alkylamino, phenyl, naphthyl or a five- or six-membered saturated, partially unsaturated or aromatic heterocycle comprising one, two, three or four heteroatoms selected from the group consisting of O, N and S, or Z—Y—(CR⁷R⁸)ₚ—(CR⁵R⁶)_{q}—CR³R⁴—# where # is the bonding site to the nitrogen atom and:
R³, R⁴, R⁵, R⁶, R⁷, R⁸ are each independently hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl, C₃-C₆-cycloalkenyl, C₃-C₆-halocycloalkenyl, phenyl, naphthyl or a five- or six-membered saturated, partially unsaturated or aromatic heterocycle comprising one, two, three or four heteroatoms selected from the group consisting of O, N and S,
R⁵ may also, with R³ or R⁷, together with the atoms to which these radicals are bonded, form a five-, six-, seven-, eight-, nine- or ten-membered saturated or partially unsaturated ring which, as well as carbon atoms, may comprise one, two or three heteroatoms selected from the group consisting of O, N and S as ring members and/or may bear one or more substituents R^{a};
R³ with R⁴, R⁵ with R⁶, R⁷ with R⁸ may each together also form carbonyl groups by being oxygen and form spiro groups by forming a C₂-C₈-alkylene or alkenylene, alkynylene chain which may be interrupted by one, two or three heteroatoms selected from the group consisting of O, N and S;
R¹ to R⁸ may each independently bear one, two, three or four identical or different R^{a} groups;
Y is oxygen or sulfur;
Z is hydrogen, carboxyl, formyl, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, C(NR^{A})SR^{Π}, C(S)R^{Π}, C(NR^{Π})NR^{A}R^{B}, C(NR^{Π})R^{A}, C(NR^{Π})OR^{A}, C(O)NR^{A}R^{B}, C(S)NR^{A}R^{B}, C₁-C₈-alkylsulfinyl, C₁-C₈-alkylthio, C₁-C₈-alkylsulfonyl, C(O)—C₁-C₄-alkylene-NR^{A}C(NR^{Π})NR^{A}R^{B},
C(S)—C₁-C₄-alkylene-NR^{A}C(NR^{Π})NR^{A}R^{B},
C(NR^{Π})—C₁-C₄-alkylene-NR^{A}C(NR^{Π})NR^{A}R^{B}, phenyl, naphthyl, five-, six-, seven-, eight-, nine- or ten-membered saturated, partially unsaturated or aromatic heterocycle which comprises one, two, three or four heteroatoms selected from the group consisting of O, N and S and is bonded directly or via a carbonyl, thiocarbonyl, C₁-C₄-alkylcarbonyl or C₁-C₄-alkylthiocarbonyl group; where the carbon chains in the Z group may be substituted by one or more R^{b} groups;
R^{A}, R^{B} are each independently hydrogen, C₂-alkenyl, C₂-alkynyl or one of the groups mentioned for R^{Π};
R^{A} and R^{B}, together with the nitrogen atom to which they are bonded, or R^{A} and R^{Π} together with the carbon atoms and heteroatoms via which they are bonded, may also form a five- or six-membered saturated, partly unsaturated or aromatic ring which, as well as carbon atoms, may comprise one, two or three further heteroatoms selected from the group consisting of O, N and S as ring members and/or may bear one or more substituents R^{a};
or
Z may also, with R⁶ or R⁸, form a five- or six-membered saturated or partly unsaturated ring which, as well as carbon atoms and Y, may comprise one or two further heteroatoms selected from the group consisting of N and S as ring members and/or may bear one or more substituents R^{a} as defined below;
the Z group may be partially or completely halogenated and/or bear one, two or three R^{b} groups;
W is phenyl or five- or six-membered heteroaryl which, as well as carbon atoms, comprises one, two or three further heteroatoms selected from the group consisting of O, N and S as ring members, where the ring systems, as well as Lₘ groups, bear at least one substituent P¹,
P¹ is Y¹—Y²-T;
Y¹ is CR^{a}R^{a′}, C(O)O, C(O)NR^{b}, O, NR^{b} or S(O)ᵣ;
Y² is C₃-C₈-alkylene, C₂-C₈-alkenylene, C₂-C₈-alkynylene, C₃-C₈-alkylene-(Y³—(C₂-C₄-alkylene))ₛ, C₃-C₈-alkenylene-(Y³—(C₂-C₄-alkylene))ₛ, C₃-C₈-alkynylene-(Y³—(C₂-C₄-alkylene))ₛ, and, if R is C₃-C₆-cycloalkyl or C₃-C₁₂-halocycloalkyl and/or Y¹ is CR^{a}R^{a′}, C(O)O, CONR^{b}, NR^{b} or S(O)ᵣ, is also C₁-C₂-alkylene, and/or, if T is OC(O)R^{a}, NR^{b}R^{b′}, C(O)NR^{b}R^{b′}, C(NOR^{c})R^{a} or T¹-C(=T²)-T³, is also C₁-C₂-alkylene;
Y³ is O, S, or NR^{b}
r is 0, 1 or 2 is 1 or 2,
T is OR^{c}, OC(O)R^{a}, NR^{b}R^{b′}, C(O)NR^{b}R^{b′}, C(NOR^{c})R^{a}, or T¹-C(=T²)-T³, and, if R is defined as NR¹R² or C₃-C₁₂-halocycloalkyl, is also C(O)OR^{c} or C(OR^{c})₂R^{a};
T¹ is O, or NR^{b};
T² is O, S, or NR^{b};
T³ is R^{a}, OR^{c}, SR^{c}, or NR^{b}R^{b′},
where R^{a′} and R^{b}, independently of R^{a} and R^{b} respectively, are each as defined for these radicals;
L is halogen, hydroxyl, cyanato (OCN), cyano, nitro, C₁-C₈-alkyl, C₁-C₈-haloalkyl, C₂-C₁₀-alkenyl, C₂-C₁₀-haloalkenyl, C₂-C₁₀-alkynyl, C₃-C₆-cycloalkyl, C₃-C₆-halocycloalkyl, C₃-C₆-cycloalkenyl, C₁-C₈-alkoxy, C₁-C₈-haloalkoxy, C₂-C₁₀-alkenyloxy, C₂-C₁₀-alkynyloxy, C₃-C₆-cycloalkyloxy, C₃-C₆-cycloalkenyloxy, amino, C₁-C₄-alkylamino, di-(C₁-C₄)-alkylamino, C₁-C₄-alkylcarbonylamino, C(O)—R^{Φ}, C(S)—R^{Φ}, S(O)ₙ—R^{Φ}; C₁-C₈-alkoxyimino-(C₁-C₈)-alkyl, C₂-C₁₀-alkenyloxyimino-(C₁-C₈)-alkyl, C₂-C₁₀-alkynyloxyimino-(C₁-C₈)-alkyl, C₂-C₁₀-alkynylcarbonyl, C₃-C₆-cycloalkylcarbonyl, or a five-, six-, seven-, eight-, nine- or ten-membered saturated, partly unsaturated or aromatic heterocycle comprising one, two, three or four heteroatoms selected from the group consisting of O, N and S;
R^{Φ} is hydrogen, C₁-C₄-alkyl, C₁-C₂-haloalkyl, C₁-C₄-alkoxy, C₂-C₄-alkenyloxy, C₂-C₄-alkynyloxy, amino, C₁-C₄-alkylamino, or di-C₁-C₄-alkylamino; where the R¹ groups may be substituted by one, two or three identical or different R^{b} groups as defined above;
n is zero, 1 or 2;
m is zero, 1, 2, 3, 4 or 5 and
X is F, I, cyano, C₁-C₄-alkyl, C₁-C₄-haloalkyl, C₁-C₄-alkoxy or C₁-C₄-haloalkoxy.

42: A compound of formula I in which 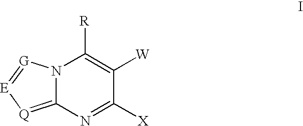
or an agriculturally acceptable salt thereof, where the substituents in the formula (I) are each defined as follows:
G, E, Q a)G is N; E is C—W² and Q is N or C—W³;
b) G is C—W¹; E is C—W² and Q is N; or
c) G is C—W¹; E is N and Q is C—W³;
W¹, W², W³ are each independently selected from the group consisting of hydrogen, halogen, cyano, nitro, C₁-C₄-alkyl, C₂-C₆-alkenyl, C₂-C₆-alkynyl, C₁-C₄-haloalkyl, hydroxy-C₁-C₄-alkyl, C₁-C₄-alkoxy-C₁-C₄-alkyl, C₂-C₆-haloalkenyl, C₂-C₆-haloalkynyl, C₃-C₆-cycloalkyl, C₃-C₆-halocycloalkyl, C₁-C₄-alkoxy, C₁-C₄-haloalkoxy, C₁-C₄-alkylthio, C₁-C₄-alkylsulfinyl, C₁-C₄-alkylsulfonyl, formyl, thiocarbamoyl, C₁-C₄-alkylcarbonyl, C₁-C₄-alkoxycarbonyl, C₁-C₄-alkylaminocarbonyl, aminocarbonyl, di(C₁-C₄-alkyl)aminocarbonyl, C₁-C₄-alkoximinoalkyl, hydroximinoalkyl, and CR¹⁰R¹¹OR², C(R¹³)═NR¹⁴;
R¹⁰, R¹¹, R¹² are each independently hydrogen, C₁-C₈-alkyl, C₃-C₆-cycloalkyl, C₁-C₈-alkoxy-C₁-C₈-alkyl, C₂-C₈-alkenyl, C₂-C₈-alkynyl, or benzyl;
R¹¹ and R¹² may together be oxy-C₁-C₅-alkyleneoxy in which the carbon chain may be substituted by from one to three groups selected from the group consisting of methyl, ethyl, hydroxyl, methoxy, ethoxy, hydroxymethyl, methoxymethyl, and ethoxymethyl;
R¹³ is hydrogen or C₁-C₈-alkyl;
R¹⁴ is C₁-C₈-alkyl, C₃-C₆-cycloalkyl, phenyl, or phenylamino, where the phenyl groups may be substituted by from one to five R^{b} groups;
R is C₃-C₁₂-halocycloalkyl; where R may comprise one, two, three or four identical or different R^{a} groups which are each independently selected from:
R^{a} is R^{b}, carboxyl, OC(O)OR^{Π} or C₁-C₆-alkylthio;
R^{b} is R^{c}, hydroxyl, C₁-C₆-alkoxy, C₂-C₆-alkenyloxy, C₃-C₆-alkynyloxy, C₃-C₆-cycloalkoxy or C₃-C₆-cycloalkenyloxy;
R^{c} is hydrogen, cyano, C₁-C₆-alkyl, C₂-C₆-alkenyl, C₂-C₆-alkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, amino, C₁-C₆-alkylamino, di-C₁-C₆-alkylamino, aminocarbonyl, C(O)NHR^{Π}, C(O)NR^{Π}₂, phenyl, naphthyl, five-, six-, seven-, eight-, nine- or ten-membered saturated, partially unsaturated or aromatic heterocycle comprising one, two, three or four heteroatoms selected from the group consisting of O, N and S,
or two R^{a}, R^{b}, R^{C}, R^{Π}, R^{d} and/or R^{dd} groups form, together with the atom or the atoms to which they are bonded, a 3- to 12-membered saturated, partially unsaturated or aromatic ring which is carbocyclic or comprises from one to four heteroatoms selected from the group consisting of N, O and S and which is unsubstituted or substituted by from 1 to 4 R^{d} radicals, in the case of halogen even up to the maximum number;
R^{Π} is C₁-C₈-alkyl, C₃-C₈-alkenyl, C₃-C₈-alkynyl, C₃-C₆-cycloalkyl or C₃-C₆-cycloalkenyl;
where the aliphatic, alicyclic or aromatic groups in the aforementioned R^{a}, R^{b}, R^{c} and R^{Π} groups may in turn be partially or fully halogenated and/or bear one, two or three R^{d} groups:
R^{d} is halogen, cyano, nitro, hydroxyl, mercapto, amino, carboxyl, alkyl, haloalkyl, alkenyl, alkynyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms; cycloalkyl, cycloalkoxy, heterocyclyl, heterocyclyloxy, where the cyclic systems comprise from 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals comprise preferably from 6 to 10 ring members, the hetaryl radicals 5 or 6 ring members, where the cyclic systems are partially or completely halogenated and/or may bear from 1 to 3 R^{dd} groups;
R^{dd} is halogen, cyano, nitro, hydroxyl, mercapto, amino, carboxyl, alkyl, haloalkyl, alkenyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, oxo (═O), thioxo (═S), C₁-C₈-alkylimino (═N—C₁-C₈-alkyl), C₁-C₈-alkoximino (═N—O—C₁-C₈-alkyl), C₃-C₈-alkenyloximino (═N—O—C₃-C₈-alkenyl), C₃-C₈-alkynyloximino (═N—O—C₃-C₈-alkynyl), where the alkyl groups in these radicals comprise from 1 to 6 carbon atoms and the alkenyl or alkynyl groups in these radicals comprise from 2 to 8 carbon atoms;
R¹ is C₁-C₁₂-haloalkyl, C₂-C₁₂-haloalkenyl, or C₂-C₁₂-haloalkynyl;
R² is H, R¹, C₁-C₁₂-alkyl, C₂-C₁₂-alkenyl, C₂-C₁₂-alkynyl, C₃-C₈-cycloalkyl, C₃-C₈-halocycloalkyl, C₃-C₆-cycloalkenyl, C₃-C₆-halocycloalkenyl, C₁-C₈-alkoxy, C₂-C₈-alkenyloxy, C₂-C₈-alkynyloxy, C₃-C₈-cycloalkoxy, NH₂, C₁-C₈-alkylamino, di-C₁-C₈-alkylamino, phenyl, naphthyl or a five- or six-membered saturated, partially unsaturated or aromatic heterocycle comprising one, two, three or four heteroatoms selected from the group consisting of O, N and S, or Z—Y—(CR⁷R⁸)ₚ—(CR⁵R⁶)_{q}—CR³R⁴—# where # is the bonding site to the nitrogen atom and:
R³, R⁴, R⁵, R⁶, R⁷, R⁸ are each independently hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl, C₃-C₆-cycloalkenyl, C₃-C₆-halocycloalkenyl, phenyl, naphthyl or a five- or six-membered saturated, partially unsaturated or aromatic heterocycle comprising one, two, three or four heteroatoms selected from the group consisting of O, N and S,
R⁵ may also, with R³ or R⁷, together with the atoms to which these radicals are bonded, form a five-, six-, seven-, eight-, nine- or ten-membered saturated or partially unsaturated ring which, as well as carbon atoms, may comprise one, two or three heteroatoms selected from the group consisting of O, N and S as ring members and/or may bear one or more substituents R^{a};
R³ with R⁴, R⁵ with R⁶, R⁷ with R⁸ may each together also form carbonyl groups by being oxygen and form spiro groups by forming a C₂-C₅-alkylene or alkenylene, alkynylene chain which may be interrupted by one, two or three heteroatoms selected from the group consisting of O, N and S;
R¹ to R⁸ may each independently bear one, two, three or four identical or different R^{a} groups;
Y is oxygen or sulfur;
Z is hydrogen, carboxyl, formyl, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, C(NR^{A})SR^{Π}, C(S)R^{Π}, C(NR^{Π})NR^{A}R^{B}, C(NR^{Π})R^{A}, C(NR^{Π})OR^{A}, C(O)NR^{A}R^{B}, C(S)NR^{A}R^{B}, C₁-C₈-alkylsulfinyl, C₁-C₈-alkylthio, C₁-C₈-alkylsulfonyl, C(O)—C₁-C₄-alkyleneNR^{A}C(NR^{Π})NR^{A}R^{B},
C(S)—C₁-C₄-alkylene-NR (NR^{Π})NR^{A}R^{Π}
C(NR^{Π})-C₁-C₄-alkylene-NR^{A}C(NR^{Π})NR^{A}R^{B}, phenyl, naphthyl, five-, six-, seven-, eight-, nine- or ten-membered saturated, partially unsaturated or aromatic heterocycle which comprises one, two, three or four heteroatoms selected from the group consisting of O, N and S and is bonded directly or via a carbonyl, thiocarbonyl, C₁-C₄-alkylcarbonyl or C₁-C₄-alkylthiocarbonyl group; where the carbon chains in the Z group may be substituted by one or more R^{b} groups;
R^{A}, R^{B} are each independently hydrogen, C₂-alkenyl, C₂-alkynyl or one of the groups mentioned for R^{Π};
R^{A} and R^{B}, together with the nitrogen atom to which they are bonded, or R^{A} and R^{Π} together with the carbon atoms and heteroatoms via which they are bonded, may also form a five- or six-membered saturated, partly unsaturated or aromatic ring which, as well as carbon atoms, may comprise one, two or three further heteroatoms selected from the group consisting of O, N and S as ring members and/or may bear one or more substituents R^{a};
or
Z may also, with R⁶ or R⁸, form a five- or six-membered saturated or partly unsaturated ring which, as well as carbon atoms and Y, may comprise one or two further heteroatoms selected from the group consisting of N and S as ring members and/or may bear one or more substituents R^{a} as defined below;
the Z group may be partially or completely halogenated and/or bear one, two or three R^{b} groups;
W is phenyl or five- or six-membered heteroaryl which, as well as carbon atoms, comprises one, two or three further heteroatoms selected from the group consisting of O, N and S as ring members, where the ring systems, as well as Lₘ groups, bear at least one substituent P¹,
P¹ is Y¹—Y²-T;
Y¹ is CR^{a}R^{a′}, C(O)O, C(O)NR^{b}, O, NR^{b} or S(O)ᵣ;
Y² is C₃-C₈-alkylene, C₂-C₈-alkenylene, C₂-C₈-alkynylene, C₃-C₈-alkylene-(Y³—(C₂-C₄-alkylene))ₛ, C₃-C₈-alkenylene-(Y³—(C₂-C₄-alkylene))ₛ, C₃-C₈-alkynylene-(Y³—(C₂-C₄-alkylene))ₛ, and, if R is C₃-C₆-cycloalkyl or C₃-C₁₂-halocycloalkyl and/or Y¹ is CR^{a}R^{a′}, C(O)O, CONR^{b}, NR^{b} or S(O)ᵣ, is also C₁-C₂-alkylene, and/or, if T is OC(O)R^{a}, NR^{b}R^{b′}, C(O)NR^{b}R^{b′}, C(NOR^{c})R^{a or T}¹-C(=T²)-T³, is also C₁-C₂-alkylene;
Y³ is O, S, or NR^{b}
r is 0, 1 or 2
s 1 or 2,
T is OR^{c}, OC(O)R^{a}, NR^{b}R^{b′}, C(O)NR^{b}R^{b′}, C(NOR^{c})R^{a}, or T¹-C(=T²)-T³, and, if R is defined as NR¹R² or C₃-C₁₂-halocycloalkyl, is also C(O)OR^{c} or C(OR^{c})₂R^{a};
T¹ is O, or NR^{b};
T² is O, S, or NR^{b};
T³ is R^{a}, OR^{c}, SR^{c}, or NR^{b}R^{b′},
where R^{a′} and R^{b′}, independently of R^{a} and R^{b} respectively, are each as defined for these radicals;
L is halogen, hydroxyl, cyanato (OCN), cyano, nitro, C₁-C₈-alkyl, C₁-C₈-haloalkyl, C₂-C₁₀-alkenyl, C₂-C₁₀-haloalkenyl, C₂-C₁₀-alkynyl, C₃-C₆-cycloalkyl, C₃-C₆-halocycloalkyl, C₃-C₆-cycloalkenyl, C₁-C₈-alkoxy, C₁-C₈-haloalkoxy, C₂-C₁₀-alkenyloxy, C₂-C₁₀-alkynyloxy, C₃-C₆-cycloalkyloxy, C₃-C₆-cycloalkenyloxy, amino, C₁-C₄-alkylamino, di-(C₁-C₄)-alkylamino, C₁-C₄-alkylcarbonylamino, C(O)—R^{Φ}, C(S)—R^{Φ}, S(O)ₙR^{Φ}; C₁-C₈-alkoxyimino-(C₁-C₈)-alkyl, C₂-C₁₀-alkenyloxyimino-(C₁-C₈)-alkyl, C₂-C₁₀-alkynyloxyimino-(C₁-C₈)-alkyl, C₂-C₁₀-alkynylcarbonyl, C₃-C₆-cycloalkylcarbonyl, or a five-, six-, seven-, eight-, nine- or ten-membered saturated, partly unsaturated or aromatic heterocycle comprising one, two, three or four heteroatoms selected from the group consisting of O, N and S;
R^{Φ} is hydrogen, C₁-C₄-alkyl, C₁-C₂-haloalkyl, C₁-C₄-alkoxy, C₂-C₄-alkenyloxy, C₂-C₄-alkynyloxy, amino, C₁-C₄-alkylamino, or di-C₁-C₄-alkylamino; where the R^{Φ} groups may be substituted by one, two or three identical or different R^{b} groups as defined above;
n is zero, 1 or 2;
m is zero, 1, 2, 3, 4 or 5 and
X is halogen, cyano, C₁-C₄-alkyl, C₁-C₄-haloalkyl, C₁-C₄-alkoxy or C₁-C₄-haloalkoxy.

43: A composition comprising a solid or liquid carrier and a compound of claim 37.

44: A composition comprising a solid or liquid carrier and a compound of claim 38.

45: A composition comprising a solid or liquid carrier and a compound of claim 39.

46: A composition comprising a solid or liquid carrier and a compound of claim 40.

47: A composition comprising a solid or liquid carrier and a compound of claim 41.

48: A composition comprising a solid or liquid carrier and a compound of claim 42.
